(19) United States
(12) Patent Application Publication
(10) Pub. No.: US 2026/0125678 A1
(43) Pub. Date: May 7, 2026

(54) DNA IMMORTALIZATION CONSTRUCT AND PERFORMING PRIMARY CELL IMMORTALIZATION

(71) Applicant: Government of the United States of America, as represented by the Secretary of Commerce, Gaithersburg, MD (US)

(72) Inventors: Zhiyong He, North Potomac, MD (US); Hua-Jun He, Silver Spring, MD (US); Kenneth D. Cole, Gaithersburg, MD (US)

(21) Appl. No.: 19/400,315

(22) Filed: Nov. 25, 2025

Related U.S. Application Data
(60) Provisional application No. 63/716,281, filed on Nov. 5, 2024.

(51) Int. Cl. C12N 15/113; C12N 5/0783; C12N 9/16; C12N 15/90; C12P 19/28

(52) U.S. Cl. CPC C12N 15/113; C12N 5/0636; C12N 9/16; C12N 15/907; C12P 19/28; C12Y 301/21004; C12N 2800/30; C12N 2840/203

(57) ABSTRACT

A DNA immortalization construct includes a first nucleic acid sequence derived from the 5′ end of human CDKN2A gene exon 2. An EF1α promoter sequence is connected to the first sequence. A first LoxP sequence is connected to the EF1α promoter sequence. A human TERT gene sequence is connected to the first LoxP sequence. A second LoxP sequence is connected to the human TERT gene sequence. An internal ribosomal entrance site (IRES) sequence is connected to the second LoxP sequence. An antibiotic selection gene sequence is connected to the IRES sequence. An SV40 poly-A signal sequence is connected to the antibiotic selection gene sequence. A second nucleic acid sequence is connected to the SV40 poly-A signal sequence and derived from 3′ end of human CDKN2A gene exon 2.

### CROSS-REFERENCE TO RELATED APPLICATION

This application claims the benefit of U.S. Provisional Patent Application Ser. No. 63/716,281 (filed Nov. 5, 2024), which is herein incorporated by reference in its entirety.

### STATEMENT REGARDING FEDERALLY SPONSORED RESEARCH

This invention was made with United States Government support from the National Institute of Standards and Technology (NIST), an agency of the United States Department of Commerce. The Government has certain rights in this invention.

### REFERENCE TO AN ELECTRONIC SEQUENCE LISTING

The contents of the electronic sequence listing (21-066P1.xml; Size: 5200 bytes; and date of creation: Oct. 16, 2024) is herein incorporated by reference in its entirety.

### BACKGROUND

The present invention generally relates to the field of genetic engineering and cell biology, and more particularly to techniques for immortalizing primary cells using a targeted DNA construct.

The ability to culture and maintain viable cell lines in vitro is involved for a wide range of biological research and commercial applications, including drug discovery, vaccine development, and the production of biologics. Primary cells, harvested directly from living tissue, offer the advantage of reflecting the in vivo characteristics of their source, but they also present significant limitations. Primary cells typically have a finite replicative lifespan, undergoing senescence after a limited number of cell divisions. This Hayflick limit poses a significant obstacle to large-scale studies and commercial production, as it necessitates repeated harvesting of fresh tissue, introducing variability and increasing costs. Existing techniques for immortalizing cell lines, such as viral transduction or transfection with oncogenes like SV40 large T-antigen, often come at the cost of altering cellular function and genetic stability. Such immortalized cell lines may exhibit abnormal karyotypes, altered gene expression profiles, and even tumorigenic properties, raising concerns about their suitability as models for in vivo processes and their reliability in research and commercial applications. The resulting trade-off between replicative capacity and phenotypic fidelity presents an ongoing challenge for researchers and industry.

Conventional approaches to cell immortalization often rely on introducing viral genes or activating endogenous oncogenes, effectively hijacking the cell's regulatory machinery to bypass normal growth control mechanisms. While these methods can achieve extended replicative lifespans, they often introduce uncontrolled genetic changes, creating cell lines that may deviate significantly from their primary cell counterparts. The random nature of viral integration and the pleiotropic effects of oncogenes can lead to unpredictable disruptions of cellular function, genetic instability, and even malignant transformation. Targeted approaches, such as introducing the catalytic subunit of telomerase (hTERT), offer a less disruptive alternative, but even these methods can be challenging to control, with variable expression levels and potential off-target effects. Furthermore, many current techniques rely on viral vectors for gene delivery, which raises additional safety concerns, especially for cell lines intended for therapeutic or clinical applications. The art therefore presents a spectrum of options, from highly disruptive but effective to more targeted but less reliable, with no single method offering both extended replicative capacity and preserved cellular function.

Precise and controlled immortalization methods are especially useful for research areas requiring stable and representative cell models. Studies of cellular differentiation, tissue engineering, and drug discovery depend on cell lines that faithfully reflect the in vivo characteristics of their source. Moreover, the growing field of personalized medicine demands cell lines that can be derived from individual patients, while retaining their unique genetic and phenotypic profiles. Conventional immortalization techniques often fall short of these requirements, introducing variability and altering cellular functions in ways that can confound research results and limit the development of new therapies. The art thus involves a more refined approach, combining the ability to extend cellular lifespan with precise control over genetic manipulation, minimal disruption of cellular function, and a high degree of reproducibility.

It is therefore an objective of the present invention to provide a method and a DNA construct for immortalizing primary cells by targeted insertion of a single copy of the hTERT gene, coupled with disruption of the CDKN2A gene, thereby overcoming the above-mentioned disadvantages of the prior art at least in part. Accordingly, methods and equipment for targeted gene insertion and homologous recombination enabling the production of stable, functionally representative immortalized cell lines would be advantageous and would be favorably received in the art.

### BRIEF DESCRIPTION

One aspect of the present invention relates to a DNA immortalization construct. A DNA immortalization construct may be understood as a synthetic DNA molecule designed to introduce specific genetic modifications into primary cells, enabling the establishment of immortalized cell lines.

It may be provided that the DNA immortalization construct comprises a first nucleic acid sequence derived from the 5′ end of human CDKN2A gene exon2. The CDKN2A gene encodes for proteins, p16 (also referred to as p16INK4a) and p14 (also referred to as p14ARF), that regulate cell cycle progression and senescence. By including a sequence derived from this gene, the construct targets the specific genomic locus associated with cellular senescence. One advantage of this arrangement is its ability to facilitate targeted gene insertion via homologous recombination. This targeted approach minimizes the risk of disrupting other genes or regulatory elements, thereby preserving the genetic integrity and functional characteristics of the immortalized cell lines. Another advantage of the specific targeting afforded by the sequence homologous to the CDKN2A gene is the potential to disrupt the CDKN2A gene itself.

It may be provided that the DNA immortalization construct comprises an EF1α promoter sequence connected to the first sequence. An EF1α promoter is a strong, constitutively active promoter derived from the human elongation factor 1 alpha gene. This promoter drives high-level expression of genes placed under its control. One advantage of this arrangement is its ability to ensure robust expression of the hTERT gene, which extends the replicative lifespan of the immortalized cells.

It may be provided that the DNA immortalization construct comprises a first LoxP sequence connected to the EF1α promoter sequence. A LoxP sequence is a specific DNA sequence recognized by the Cre recombinase enzyme. One advantage of this arrangement is that it provides a mechanism for conditionally removing the hTERT gene from the immortalized cell lines if desired. This can be useful for studying the effects of hTERT expression or for reverting the cells to a non-immortalized state.

It may be provided that the DNA immortalization construct comprises a human TERT gene sequence connected to the first LoxP sequence. The hTERT gene encodes the catalytic subunit of telomerase, an enzyme responsible for maintaining telomere length. One advantage of this arrangement is that it allows the cells to overcome telomere shortening, a factor in cellular senescence, and thereby extends their replicative lifespan, enabling the establishment of immortalized cell lines.

It may be provided that the DNA immortalization construct comprises a second LoxP sequence connected to the human TERT gene sequence. One advantage of this arrangement, combined with the first LoxP site, provides the mechanism for conditional removal of the hTERT gene described above.

It may be provided that the DNA immortalization construct comprises an internal ribosomal entrance site (IRES) sequence connected to the second LoxP sequence. An IRES sequence allows for the translation of multiple proteins from a single mRNA transcript. One advantage of this arrangement is that it enables the simultaneous expression of both the hTERT gene and a selectable marker gene, such as an antibiotic resistance gene, from a single promoter. This simplifies the construct design and ensures that cells expressing hTERT also express the selectable marker.

It may be provided that the DNA immortalization construct comprises an antibiotic selection gene sequence connected to the IRES sequence. One advantage of this arrangement is its ability to facilitate the selection and isolation of cells that have successfully integrated the DNA construct. By culturing the transfected cells in the presence of the corresponding antibiotic, only cells expressing the resistance gene, and therefore also expressing hTERT, will survive, enabling the efficient establishment of an immortalized cell line.

It may be provided that the DNA immortalization construct comprises an SV40 poly-A signal sequence connected to the antibiotic selection gene sequence. An SV40 poly-A signal is a DNA sequence that directs the addition of a poly-A tail to the 3′ end of mRNA transcripts. One advantage of this arrangement is that it enhances the stability and translation efficiency of the mRNA transcripts encoding the hTERT and antibiotic resistance proteins, thereby further increasing the effectiveness of the immortalization and selection processes.

It may be provided that the DNA immortalization construct comprises a second nucleic acid sequence connected to the SV40 poly-A signal sequence and derived from the 3′ end of human CDKN2A gene exon 2. One advantage of this arrangement is its ability to facilitate targeted gene insertion through homologous recombination, similar to the first sequence derived from the 5′ end of the CDKN2A gene, as discussed above.

One aspect of the present invention relates to a method for immortalizing primary cells.

It may be provided that the method comprises providing a DNA immortalization construct comprising various components. One advantage of providing such a construct is that it provides a targeted approach to cell immortalization, enabling precise control over the genetic modifications introduced into the cells.

It may be provided that the method comprises isolating primary cells from human tissue. Primary cells can be cells harvested directly from living tissue. One advantage of using primary cells is that they retain the in vivo characteristics of their source, making them valuable for research and commercial applications.

It may be provided that the method comprises culturing the primary cells to produce recipient cells. Culturing cells involves maintaining them in vitro under controlled conditions that promote cell growth and proliferation. One advantage of this step is that it generates a sufficient number of cells for subsequent transfection and immortalization.

It may be provided that the method comprises transfecting the recipient cells with the DNA immortalization construct using homologous recombination to produce transfected cells. Transfection is the process of introducing exogenous DNA into cells. Homologous recombination is a type of genetic recombination in which nucleotide sequences are exchanged between two similar or identical molecules of DNA. One advantage of using homologous recombination is that it allows for precise targeting of the DNA immortalization construct to a specific location in the genome, such as the CDKN2A locus. It should be appreciated CDKN2A is also referred to as the INK4a/ARF locus, which is a tumor suppressor gene on chromosome 9p21 that encodes two proteins, p16INK4a and p14ARF.

It may be provided that the method comprises culturing the transfected cells in a medium comprising an antibiotic to select for transfected cells exhibiting antibiotic resistance. One advantage of this selection step is that it isolates cells that have successfully integrated the DNA immortalization construct and are therefore expressing the desired genes, including the hTERT gene and the antibiotic resistance gene.

It may be provided that the method comprises isolating the transfected cells exhibiting resistance to the antibiotic to produce an immortalized cell line. An immortalized cell line is a population of cells capable of continuous cell division in vitro. One advantage of establishing an immortalized cell line is that it provides a consistent and readily available source of cells for research, development, and commercial applications.

### BRIEF DESCRIPTION OF THE DRAWINGS

The following description cannot be considered limiting in any way. Various objectives, features, and advantages of the disclosed subject matter can be more fully appreciated with reference to the following detailed description of the disclosed subject matter when considered in connection with the following drawings, in which like reference numerals identify like elements.

FIG. 1 shows, according to some embodiments, a DNA immortalization construct for an hTERT knock-in targeting vector.

FIG. 2 shows, according to some embodiments, a targeting vector and p16 gene before and after homologous recombination.

FIG. 3 shows, according to some embodiments, sequence details for a DNA immortalization construct that can be used as a universal human primary cell immortalization vector.

FIG. 4 shows, according to some embodiments, a process for performing primary cell immortalization.

FIG. 5 shows, according to some embodiments, immortalization of human primary CD8+ T cells, including (A) a schematic view of donor DNA design and knockout p16 exon 2 after recombination, (B) growth of immortalized CD8+T-TERT cells, (C) copy numbers of p16 exon2, exon 1, TERT transgene and IRES in the immortalized CD8+T-TERT cells, (D) detection of recombinant p16 allele in CD8+T-TERT cells by PCR, and (E) detection of p16 WT and KO alleles by PCR.

FIG. 6 shows, according to some embodiments, relative p16 and TERT mRNA expression in hCD8+T-TERT cells.

FIG. 7 shows, according to some embodiments, the karyotype of hCD8+T-TERT cells.

FIG. 8 shows, according to some embodiments, that immortalized T cells retained dependence of IL-2 and CD3/CD28 activator.

FIG. 9 shows, according to some embodiments, T cell surface markers expression.

FIG. 10 shows, according to some embodiments, T cell differentiation markers expression.

FIG. 11 shows, according to some embodiments, T cell exhaustion and activation surface markers expression.

FIG. 12 shows, according to some embodiments, a schematic view of genotyping primers design.

FIG. 13 shows, according to some embodiments, a growth curve of immortalized hMEPC.

FIG. 14 shows, according to some embodiments, amplification of a DNA fragment in the recombinant allele by recombinant specific allele primers.

FIG. 15 shows, according to some embodiments, PCR results using WT and KO specific primers revealing p16 exon2 KO status.

FIG. 16 shows, according to some embodiments, gene copy numbers detected by ddPCR.

FIG. 17 shows, according to some embodiments, Tert and p16 mRNA level expression in parental WT epithelial cells and immortalized clonal cell lines.

FIG. 18 shows, according to some embodiments, epithelial cell marker gene expression on mRNA level by RT-ddPCR.

FIG. 19 shows, according to some embodiments, FACS analysis of epithelial cell surface markers.

### DETAILED DESCRIPTION

A detailed description of one or more embodiments is presented herein by way of exemplification and not limitation.

Establishing and maintaining viable cell lines are useful for biological research, drug discovery, and the production of biologics. Conventional methods for immortalizing primary cells, such as viral transduction or transfection with oncogenes, often result in significant alterations to cellular function, gene expression, and genomic stability. These uncontrolled changes can compromise the reliability and reproducibility of experimental results, limiting the utility of immortalized cell lines, particularly in applications requiring accurate models of in vivo processes. The random nature of viral integration and the pleiotropic effects of oncogenes can lead to unpredictable and undesirable outcomes, including tumorigenic transformation and karyotypic abnormalities. Furthermore, many existing methods lack the precision needed for targeted genetic manipulation, hindering the development of cell lines with specific modifications, involved for advanced research areas like personalized medicine and regenerative therapies.

The DNA immortalization construct overcomes these limitations by providing a targeted and controlled approach to cell immortalization. It has been discovered that a DNA immortalization construct comprising specific sequences enables the precise insertion of the hTERT gene into a pre-determined genomic locus, coupled with disruption of the CDKN2A gene. One advantage of this construct is its ability to minimize off-target effects and preserve genomic stability, thereby producing immortalized cell lines that retain the functional characteristics of their primary cell counterparts. The inclusion of homologous arms derived from the CDKN2A gene allows for targeted insertion via homologous recombination, avoiding the random integration associated with viral vectors and reducing the risk of disrupting involved genes or regulatory elements. The targeted insertion not only introduces the hTERT gene, involved for overcoming telomere shortening and cellular senescence, but also disrupts the CDKN2A gene, a regulator of cell cycle progression and senescence, further enhancing the immortalization process. Moreover, the inclusion of LoxP sites flanking the hTERT gene enables conditional removal of the gene if desired, providing a mechanism for reverting the immortalized cells to a non-immortalized state or for studying the specific effects of hTERT expression. This combination of targeted insertion, dual-gene manipulation, and conditional control provides a level of precision and flexibility not found in conventional immortalization methods. The DNA immortalization construct provides a significant advantage in producing reliable, stable, and functionally representative cell lines for a variety of research and commercial applications.

In an embodiment, a DNA immortalization construct (200) comprises a first nucleic acid sequence (202) derived from the 5′ end of human CDKN2A gene exon 2, an EF1α promoter sequence (204) connected to the first sequence (202), a first LoxP sequence (206) connected to the EF1α promoter sequence (204), a human TERT gene sequence (208) connected to the first LoxP sequence (206), a second LoxP sequence (210) connected to the human TERT gene sequence (208), an internal ribosomal entrance site (IRES) sequence (212) connected to the second LoxP sequence (210), an antibiotic selection gene sequence (214) connected to the IRES sequence (212), an SV40 poly-A signal sequence (216) connected to the antibiotic selection gene sequence (214), and a second nucleic acid sequence (218) connected to the SV40 poly-A signal sequence (216) and derived from the 3′ end of human CDKN2A gene exon 2. In an embodiment, the first sequence (202) comprises 4.8 kilobases. In an embodiment, the antibiotic selection gene sequence (214) is a Zeocin resistance gene sequence. In an embodiment, the second sequence (218) comprises 2.1 kilobases. In an embodiment, the DNA immortalization construct (200) further comprises a vector backbone sequence (220) connected to the first sequence (202) and the second sequence (218). In an embodiment, the vector backbone sequence (220) is selected to promote homologous recombination. In an embodiment, the construct (200) is a synthetic plasmid. In an embodiment, the IRES sequence (212) is positioned between the human TERT gene sequence (208) and the antibiotic resistance gene sequence (214). In an embodiment, the first LoxP sequence (206) and the second LoxP sequence (210) allow removal of the human TERT gene sequence (208) by CRE-recombination. In an embodiment, the DNA immortalization construct (200) comprises the base sequence of SEQ ID NO: 9.

The DNA immortalization construct (200) provides a targeted approach for generating stable, functionally representative immortalized cell lines. Conventional methods, such as viral transduction with oncogenes, often disrupt cellular function and genomic stability due to the random nature of gene insertion and the pleiotropic effects of oncogenes. The construct (200), by using specific DNA sequences and homologous recombination, offers a more precise method for manipulating gene expression and extending cellular lifespan.

The first nucleic acid sequence (202), derived from the 5′ end of the human CDKN2A gene exon 2, serves as a homologous arm for targeted insertion. This sequence facilitates homologous recombination with the genomic CDKN2A locus, ensuring that the construct integrates at a specific, pre-determined site. This targeted approach minimizes the risk of insertional mutagenesis and preserves the genetic integrity of the immortalized cell lines. Variations in the length of this homologous arm are possible, with sequences ranging from several hundred base pairs to several kilobases. An alternative approach could involve using a different genomic locus for targeted insertion, but the CDKN2A locus can be used due to its role in cell cycle regulation and senescence. In an embodiment, a 4.8 kb 5′ homologous arm targets the CDKN2A locus in human primary fibroblasts.

The EF1α promoter sequence (204), connected to the first sequence (202), drives the expression of the downstream genes. The EF1α promoter is a strong, constitutively active promoter that ensures high-level transcription of the hTERT gene. This robust expression of hTERT maintains telomere length and extends the replicative lifespan of the cells. Alternative promoters, such as CMV or CAG, could be used, but EF1α is preferred due to its consistent activity in a wide range of cell types. Variations in the EF1α promoter sequence itself are possible, but the core promoter region is preserved to maintain its activity. An example would be using the EF1α promoter to drive hTERT expression in human primary epithelial cells.

The first LoxP sequence (206), connected to the EF1α promoter sequence (204), provides a site for Cre-mediated recombination. This sequence, in conjunction with the second LoxP sequence (210), enables the conditional removal of the hTERT gene (208). This feature offers flexibility for the controlled study of hTERT's effects on cellular function or for the generation of cell lines with a finite lifespan. The specific placement and orientation of the LoxP sequence are useful for efficient recombination, but variations in the surrounding sequences are possible. An alternative approach could involve using a different recombination system, such as Flp/FRT, but LoxP/Cre is widely used and readily available. LoxP sites can create a conditionally immortalized human induced pluripotent stem cell line, where hTERT expression can be turned off upon differentiation.

The human TERT gene sequence (208), connected to the first LoxP sequence (206), encodes the catalytic subunit of telomerase. This gene extends the replicative lifespan of primary cells by maintaining telomere length. Variations in the hTERT sequence are possible, including the use of codon-optimized versions or the inclusion of specific tags or mutations for research purposes, but the core catalytic domain is preserved for telomerase activity. Alternative approaches to extending cellular lifespan exist, such as inhibiting telomere-associated proteins, but direct hTERT expression is often more effective. An example includes introducing the hTERT gene into human primary keratinocytes to generate an immortalized cell line for studying skin biology.

The second LoxP sequence (210) flanks the 3′ end of the hTERT gene (208). Its function is identical to that of the first LoxP site, providing a mechanism for Cre-mediated removal of the hTERT as described above.

The internal ribosomal entry site (IRES) sequence (212), connected to the second LoxP sequence (210), allows for the co-expression of multiple genes from a single mRNA transcript. This sequence enables the simultaneous translation of both the hTERT gene and a downstream antibiotic resistance gene, simplifying the construct design and ensuring that all cells expressing hTERT also express the selectable marker. Alternative approaches to co-expression, such as using separate promoters for each gene, are possible, but the IRES sequence is compact and efficient. Variations in the IRES sequence are possible, with different IRES elements exhibiting varying degrees of translational efficiency. An example includes using an IRES element to co-express hTERT and a fluorescent protein for visualizing the transfected cells.

The antibiotic selection gene sequence (214), connected to the IRES sequence (212), confers resistance to a specific antibiotic. This gene enables the selection and isolation of cells that have successfully integrated the DNA immortalization construct (200). Variations in the antibiotic resistance gene are possible, with different genes conferring resistance to different antibiotics. The choice of antibiotic resistance gene depends on the cell type and the available selection methods, but commonly used genes include those conferring resistance to neomycin, puromycin, or Zeocin. An example would be using a Zeocin resistance gene to select for immortalized human mesenchymal stem cells.

The SV40 poly-A signal sequence (216), connected to the antibiotic resistance gene sequence (214), enhances mRNA stability and translation. This sequence signals the addition of a poly-A tail to the 3′ end of the mRNA transcript, promoting efficient translation of the hTERT and antibiotic resistance proteins. Alternative poly-A signals could be used, but SV40 is widely employed and effective. Variations in the SV40 poly-A signal sequence itself are possible but the core signal must be preserved for proper function. An example includes increased expression levels of the target genes and comparing cells transfected with constructs containing the SV40 poly-A signal with those lacking the signal.

The second nucleic acid sequence (218), connected to the SV40 poly-A signal sequence (216) and derived from the 3′ end of the human CDKN2A gene exon 2, serves as a second homologous arm for targeted insertion. Similar to the first sequence (202), this sequence further enhances homologous recombination with the CDKN2A locus, promoting precise integration of the construct. Variations of length are possible, as are alternative locations in the CDKN2A gene. A 2.1 kb homologous arm can be used to increase the targeting efficiency of hTERT knock-in.

The DNA immortalization construct (200) can include a 4.8 kilobase first sequence (202) that provides a substantial region of homology for recombination, increasing the efficiency of targeted insertion at the CDKN2A locus. This extended sequence can be generated by chemical synthesis or by PCR amplification from a genomic DNA template. Alternative lengths are possible, but shorter sequences may reduce targeting efficiency, while longer sequences may be more challenging to synthesize or clone.

Using a Zeocin resistance gene sequence (214) as the antibiotic selection marker offers a versatile approach for selecting transfected cells. Zeocin, a member of the bleomycin/phleomycin family of antibiotics, effectively inhibits protein synthesis in both prokaryotic and eukaryotic cells. The Zeocin resistance gene encodes a protein that inactivates Zeocin, allowing transfected cells to survive in the presence of the antibiotic. Alternative selectable markers, such as those conferring resistance to puromycin or neomycin, could be employed, but Zeocin is often preferred due to its broad spectrum of activity and its ability to be used at low concentrations, minimizing potential toxicity to the cells. It is contemplated that a Zeocin resistance gene can be sued for selecting immortalized human embryonic kidney cells.

A 2.1 kilobase second sequence (218), similar to the first sequence (202), serves as a second homologous arm, increasing the efficiency of homologous recombination at the CDKN2A locus. The inclusion of two homologous arms, flanking the hTERT expression cassette, enhances targeted integration and minimizes the risk of random insertion. Alternative lengths for the 3′ homologous arm could be used, as could different locations for targeting within the CDKN2A gene.

The DNA immortalization construct (200) can be incorporated into a vector backbone sequence (220) that contains elements for propagation and manipulation of the construct in bacterial systems and, in certain cases, can improve transfection efficiency in mammalian cells. The vector backbone sequence can include features such as an origin of replication, antibiotic resistance genes for selection in bacteria and other elements necessary for efficient homologous recombination in recipient cells. Various vector backbones can be used, such as those based on pUC, pBR322, or pcDNA3.1, based on the requirements for transfection and gene expression in the target cell lines. It is contemplated that the DNA immortalization construct can be cloned into a pcDNA3.1 vector for transfection into human primary hepatocytes.

Vectors specifically designed to enhance homologous recombination provide an additional advantage to the immortalization process. These vectors often include features such as specialized recombination sequences or modifications that minimize random integration, increasing the efficiency of targeted insertion at the CDKN2A locus. One could compare transfection efficiencies using a recombination-optimized vector versus a conventional vector, demonstrating improved targeting with the specialized vector.

Constructing the DNA immortalization construct (200) as a synthetic plasmid simplifies its production and manipulation. Plasmids are circular, double-stranded DNA molecules that replicate independently in bacterial cells. They are widely used in molecular biology for cloning, gene expression, and gene editing applications. Synthetic plasmids can be produced commercially with custom-designed sequences, facilitating the precise assembly of complex constructs. Alternative formats, such as viral vectors or linear DNA fragments, are possible, but plasmids are often preferred for their ease of use and versatility. A specific example would be using a commercially synthesized plasmid containing the DNA immortalization construct for transfection into human primary endothelial cells.

Positioning the IRES sequence (212) between the human TERT gene sequence (208) and the antibiotic resistance gene sequence (214) ensures their co-expression from a single transcript. This arrangement allows for efficient selection of transfected cells, as cells expressing hTERT will also express the antibiotic resistance marker. Alternative configurations, such as placing the antibiotic resistance gene upstream of hTERT or using separate promoters, are possible but less efficient.

The presence of both the first and second LoxP sequences (206, 210) flanking the hTERT gene (208) facilitates its conditional removal. These sequences serve as recognition sites for the Cre recombinase enzyme, which catalyzes the excision of DNA between two LoxP sites. This feature allows researchers to control hTERT expression in the immortalized cell lines, enabling studies of the gene's specific effects or facilitating reversion to a non-immortalized state. Variations in the LoxP sequences themselves are possible, but the core recognition sequence must be preserved for Cre-mediated recombination. Alternative recombination systems, such as Flp/FRT, could be used, but LoxP/Cre is widely used and readily available.

It is contemplated that the entire base sequence disclosed as SEQ ID NO: 9 is included in the DNA immortalization construct (200) to provide a specific embodiment with known functionality and characteristics. This sequence has been shown to effectively immortalize various human primary cell types. While modifications to this sequence are possible, SEQ ID NO: 9 provides a readily available and validated starting point to establish immortalized cell lines. It is contemplated that SEQ ID NO: 9 is a validated sequence for producing a DNA immortalization construct for hTERT knock-in at the CDKN2A locus in human primary cells.

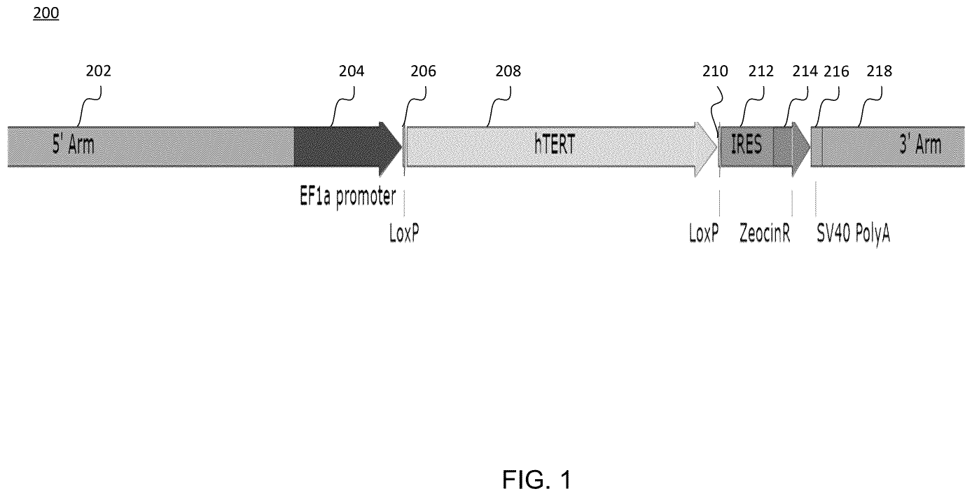

FIG. 1 illustrates a DNA immortalization construct (200) designed for targeted insertion of the hTERT gene into the human genome. The construct (200) comprises several elements arranged in a specific order to achieve efficient immortalization of primary cells. The 5′ arm (202) and 3′ arm (218), homologous to regions flanking exon 2 of the CDKN2A gene, facilitate targeted integration via homologous recombination. The EF1α promoter (204) drives high-level expression of the hTERT gene (208), involved for maintaining telomere length and extending cellular lifespan. The LoxP sites (206, 210), flanking the hTERT gene, enable its conditional removal by Cre recombinase, offering control over hTERT expression and allowing for the study of its effects or reversion to a finite lifespan. The IRES element (212) allows for the co-expression of the hTERT gene and a selectable marker, typically an antibiotic resistance gene such as Zeocin resistance (214). This co-expression strategy simplifies selection and ensures that all immortalized cells carry the hTERT gene. The SV40 polyA signal (216) enhances mRNA stability and translation efficiency, further increasing hTERT expression levels. Variations in the lengths of the homologous arms are possible, depending on the specific requirements of the target cell type and the efficiency of homologous recombination. Alternative promoters, selectable markers, and poly-A signals could be employed, but the elements depicted in FIG. 1 are useful for their demonstrated efficacy and broad applicability. It is contemplated that this construct immortalizes human primary fibroblasts by targeting the CDKN2A locus. The construct can be delivered as a linearized DNA fragment or incorporated into a plasmid vector for transfection. The choice of delivery method depends on the specific cell type and the available transfection techniques, but homologous recombination remains the central mechanism for targeted integration. The use of CRISPR/Cas9 technology for gene editing has become increasingly popular, but this method can introduce off-target effects and unwanted mutations. The DNA immortalization construct, by relying on homologous recombination, offers a more precise and controlled approach to gene insertion, minimizing the risk of unintended genetic modifications. The conditional nature of hTERT expression, controlled by the LoxP sites, provides additional flexibility for research applications, allowing for the study of hTERT's specific effects on cell function or the controlled reversion to a finite lifespan. The selection process, enabled by the selectable marker, facilitates the isolation of cells that have successfully integrated the DNA construct. This selection process is involved for the efficient establishment of immortalized cell lines. It is contemplated that that DNA immortalization construct 200 provides improved genomic stability and preserved cellular function in cell lines generated using the DNA immortalization construct. This construct can be adapted for various cell types by modifying the homologous arms to target different genomic loci or by adjusting the promoter and selectable marker for optimal expression and selection. In some embodiments, the core elements of the construct, namely the hTERT gene, LoxP sites, IRES, and poly A signal, remain consistent. The DNA immortalization construct 200 can be used to immortalize human primary epithelial cells. The DNA immortalization construct (200) thus provides a tool to generate stable, functionally relevant cell lines for diverse applications, from basic research to drug discovery and regenerative medicine. This targeted approach to immortalization allows for precise control over gene expression, minimizing disruptions to cellular function and providing a reliable platform for studying cellular processes and developing new therapies. The construct's modular design enables adaptation for various cell types and research goals, offering flexibility and control. The has superior genomic stability and preserved phenotypic characteristics in the targeted knock-in lines over conventional cell lines. The DNA immortalization construct offers a robust and versatile tool for advancing research in cell biology and regenerative medicine. Its targeted approach to gene insertion addresses limitations of existing techniques and facilitates the generation of immortalized cell lines that faithfully represent their in vivo counterparts.

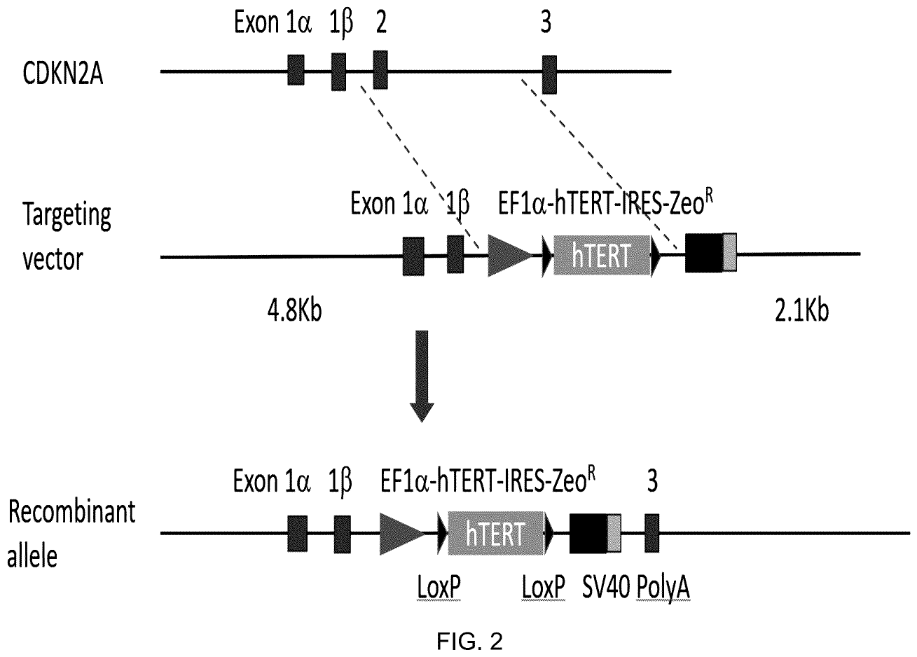

FIG. 2 show a process of homologous recombination between the DNA immortalization construct (200) and the target CDKN2A gene locus. The top portion of the figure depicts the CDKN2A gene with its exons (represented by blue boxes labeled 1, 2, and 3) and introns (represented by the line connecting the exons). The middle portion shows the targeting vector containing the DNA immortalization construct (200). The construct includes the 5′ arm (202) and the 3′ arm (218), homologous to regions flanking exon 2 of the CDKN2A gene, the EF1α promoter (204), the LoxP sites (206, 210), the hTERT gene (208), the IRES (212), and the Zeocin resistance gene (214), all integrated within the vector. The bottom portion of the figure depicts the recombinant allele resulting from homologous recombination. The DNA construct, including the hTERT expression cassette and the selectable marker, has replaced exon 2 of the CDKN2A gene. This targeted replacement results in both hTERT overexpression, driven by the EF1α promoter, and disruption of the CDKN2A gene, a regulator of cell cycle progression and senescence. The homologous recombination process ensures precise integration of the construct at the desired genomic location, minimizing off-target effects and preserving the genetic integrity of the immortalized cell lines. Homologous recombination is a fundamental process in molecular biology, employed in various gene editing and genetic engineering applications. Conventional methods for gene insertion, such as viral transduction, often result in random integration, which can disrupt involved genes or regulatory elements, leading to unpredictable and undesirable outcomes. The DNA immortalization construct's targeted approach, using homologous recombination, addresses this limitation by ensuring precise gene insertion at a predefined locus. Variations in the length and sequence of the homologous arms are possible, depending on the cell type and the efficiency of homologous recombination. Alternative targeting strategies, such as using CRISPR/Cas9 technology, exist, but homologous recombination offers increased precision and control. The resulting immortalized cell lines can be characterized by PCR and Southern blotting to confirm the precise integration of the DNA construct. These techniques verify the replacement of exon 2 with the hTERT expression cassette. PCR primers can be used for the recombinant allele to detect successful integration of the construct at the CDKN2A locus. Conventional immortalization techniques often lead to unpredictable genetic changes, potentially altering cellular function and phenotype. The DNA immortalization construct's targeted approach minimizes this risk by ensuring precise gene insertion and disruption of a specific locus associated with cellular senescence. The conditional nature of hTERT expression, controlled by the LoxP sites, provides flexibility for researchers studying the role of telomerase in cell immortalization and differentiation. This control enables them to remove the hTERT gene at specific time points, reverting the cell line to a finite lifespan. This targeted immortalization strategy offers advantages for a wide range of research applications, including drug discovery, disease modeling, and regenerative medicine. By producing immortalized cell lines with stable genomes and preserved cellular functions, researchers can more reliably study cellular processes and develop new therapies.

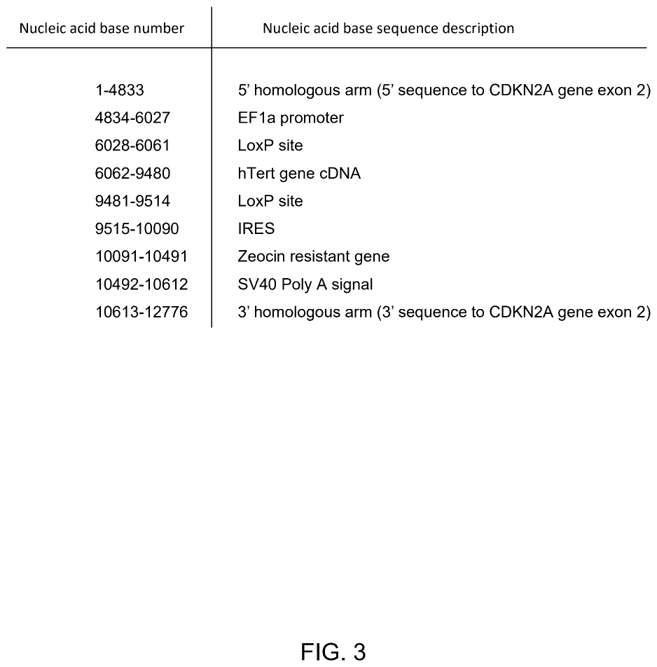

FIG. 3 provides a nucleic acid sequence of an exemplary DNA immortalization construct (200) designed for targeted insertion and expression of the hTERT gene in human primary cells. This sequence includes elements for homologous recombination, gene expression, antibiotic selection, and conditional gene removal. The 5′ arm (202), derived from the 5′ end of human CDKN2A gene exon 2, facilitates targeted integration at the CDKN2A locus through homologous recombination. The EF1α promoter (204) drives constitutive, high-level expression of the hTERT gene, ensuring robust telomerase activity and promoting cell immortalization. The LoxP sites (206, 210), flanking the hTERT coding sequence, enable Cre-mediated recombination and conditional removal of the hTERT gene. This feature allows for controlled studies of hTERT's effects or reversion to a finite lifespan. The hTERT coding sequence (208) itself encodes the catalytic subunit of telomerase, an enzyme responsible for maintaining telomere length and preventing cellular senescence. The IRES sequence (212) facilitates the co-expression of hTERT and a selectable marker, here a Zeocin resistance gene (214), from a single mRNA transcript. This simplifies the selection process and guarantees that all immortalized cells also carry the hTERT gene. The Zeocin resistance gene (214) confers resistance to the antibiotic Zeocin, allowing for the selection and isolation of successfully transfected cells. The SV40 polyA signal sequence (216) enhances mRNA stability and translation, boosting hTERT expression levels. The 3′ arm (218), homologous to the 3′ end of the CDKN2A gene exon 2, provides a second region of homology for recombination, increasing targeting efficiency. While the specific sequence provided in FIG. 3 represents a functional and validated embodiment, variations in certain elements are possible. For instance, the lengths of the homologous arms can be adjusted based on the target cell type and the efficiency of homologous recombination. Alternative promoters, selectable markers, and poly-A signals could be employed, but the elements shown in FIG. 3 can be used for their demonstrated efficacy and compatibility. In an embodiment, a DNA molecule is synthesized with this sequence using commercially available gene synthesis services. The synthesized construct can then be cloned into a suitable vector backbone for transfection into human primary cells. Accordingly, immortalization can occur for various cell types, such as fibroblasts, epithelial cells, or stem cells, using this construct. PCR analysis can confirm the precise integration of the construct at the CDKN2A locus, cell proliferation assays demonstrating extended lifespan, and phenotypic characterization of the immortalized cells, confirming the preservation of cellular functions. Conventional methods for cell immortalization, such as viral transduction with oncogenes, often introduce uncontrolled genetic changes and alter cellular function. The DNA immortalization construct, by relying on homologous recombination, offers a targeted and precise approach that minimizes these risks. The inclusion of LoxP sites provides additional control over hTERT expression, allowing researchers to manipulate telomerase activity and study its role in cellular processes. The use of Zeocin as a selectable marker facilitates the efficient establishment of immortalized cell lines by eliminating non-transfected cells. The SV40 polyA signal enhances gene expression levels, further optimizing the immortalization process. FIG. 3 therefore provides a blueprint for creating a tool for cell immortalization, enabling the generation of stable, functionally relevant cell lines for a wide range of applications. This construct's modular design enables easy modification for different cell types and experimental goals, providing researchers and manufacturers with a versatile platform for advancing their work in cell biology, regenerative medicine, and drug discovery. The detailed sequence information provided enables a skilled artisan to reproduce this construct precisely. The expression levels of hTERT in cells immortalized with this construct compared to cells immortalized by other methods shows increased and controlled hTERT expression with the targeted approach. The DNA immortalization construct offers an advance in cell culture technology, facilitating the establishment of consistent and reliable cell models for both research and commercial applications.

DNA immortalization construct 200 can be made of various elements and components that can be synthesized or produced in a biochemical environment. Elements of DNA immortalization construct 200 can be various sizes, e.g., shapes or sequence lengths.

The 5′ arm (202) of the DNA immortalization construct (200) plays a role in targeted gene insertion. This sequence, derived from the 5′ end of human CDKN2A gene exon 2, facilitates homologous recombination by providing a region of DNA identity with the target locus. Homologous recombination, a precise gene-editing mechanism, relies on the exchange of genetic material between two DNA molecules with similar sequences. The 5′ arm (202), by hybridizing with the genomic DNA at the CDKN2A locus, initiates this process and directs the integration of the entire construct into the desired location. This targeted approach minimizes the risk of insertional mutagenesis, a common concern with conventional methods like viral transduction, where the integration site is random and potentially disruptive to cellular function or gene expression. Variations in the length of the 5′ arm (202) are possible, ranging from a few hundred base pairs to several kilobases, depending on the requirements for efficient homologous recombination in the target cell type. Longer arms can provide greater specificity but may be more challenging to synthesize or clone. Alternative sequences could be employed, for example, targeting different genes or genomic loci, but the CDKN2A locus is used for its role in cell cycle regulation and senescence. A 4.8 kb 5′ arm derived from the human CDKN2A gene can be used to target the hTERT insertion in human primary fibroblasts. This approach ensures precise gene insertion and minimizes off-target effects. The targeting efficiency of constructs with different 5′ arm lengths or sequences provides benefits for optimal recombination. Conventional methods for immortalizing cells often rely on viral vectors or random integration of oncogenes, which can disrupt cellular function and gene expression. The 5′ arm's role in targeted homologous recombination offers an advantage over these methods by directing the DNA construct to a precise genomic location. This reduces the risk of insertional mutagenesis and ensures the stability of the immortalized cell lines.

In an embodiment, the 5′ arm includes a nucleic acid base sequence comprising:

(SEQ. ID NO. 1)
GGATCCGGAATAAGTCTAAATGAATCACTTTCAGTTTTCCTAAACTTCTATGCCTT
TGCACATCCTCTTACCTCTGCCTAGAATATCTTTCTCCTTCTTTTCCATCTTTAAAC
TCTCACATCATTCTTCAAGACTGGGATCAGCTCTCAGCATCCGGAAGCCTTTGCC
TACTAGAGACAAATGAGAATGAGTTTGGTCACCTTTTCATTTTCTTGTATCATTCT
GTGCTTTATTTTGCTCTTCTAAGAGCGTTACATGCTTCATTTAATCCCTAAACAAC
TGTTTGAGGCAAGTACAGTTATTATCCTAATCATGCAAATGAGAAAACAGAGGC
CCAGACATGTTGAGTAACTTTGATAAAAGTTAAAGAACCAATAAGTGGAACAGT
TGAGGTTTGAACCCTGGCAGTCTGACTGTAGAGATACTATGTTTGACCTACTCCC
CTCTGCCCCCACCCCATGTCTGCCCTTAGTTTCTGAGCTTGTTGAATGAATGAACA
GGTGGTAGTCTTTTTTTGTTATAAGACTGATCAGAATTAAGACAGGTTTAAATTTC
ACGTGTAGAATTTTCAAAACTGCAAAGGCAGTGCAAATCTAAAAAAAGAATGGC
ATTCTCAGGAAAGAGGAAAAGTAAGTGTGAGAATAATAATAACAATAACCAACA
AACTTTAGTAAATTTAGTAAATGTAGTAAATTTTTACATTAAAAGCTTTTGGACA
TACATTATCATATTTTATGGCCACATGAAATATATTATAATCCCATTTTGCACATA
GGAAATCTGAGACTGGCATAAGGAGCACAGAGATCCAGGACTTTATATTTTCATT
CTTCTAGGATTTTGCACCTCAGGTCGATATGTATGAGTAAACTGGGAGTATAATG
GGCTCTTTAACAGAAAAACTAGGAAAGTTTTCCCACTATTATTAATTATTTACAT
AATATTTTTTTAATTTTATTATTATTTATACTTTAAGTTTTAGAGTACATGTGCACA
ATGTGCAGGTTTGTTACATATGTATACATGTGCCATGTTGGTGTGCTGCACCCATC
AACTCATCATTTAGCATTAGGTATATCTCCTAATGCTATCCCTCCCCCCTCCCCCC
TACATAAGATTTATAATGGATAATGGACTTCAATTTCTAGAGCAAAATGGCCCCA
CCCAAGGATGCCATAATCCTTCCAGAGCTCTACTGCAAGATATGAGATATACATA
TCTAAAACTTGTTCTTGGTATTTCCAAAGCAGTCAACTTTTACACCTGTTTATAAT
GCATCCAAATGTTGTTTTTATATGGTTGCATCTCCCATCTTCTTCACCAATAGCTA
TATATATTTTTCACAAGAGCTGAAAGAGTTCTTGATGTAGGAATCCATGGTAGAG
TTTCAGAGAAATCCCTGAATTCACTGAAAGTTTTATCTAGAAATACATGTGCAAG
TGAACACATCTTTTTTAAAAAAAATCATTACCTACTTTCTTTTTTGAGAAGAAGGT
ATTTATTTCAACAGACTCTTGAAGGAGCCTACTCTTCCCACTCTCCCACCCCCATT
AAGAACCACTGTAGGCCGGGCACGATGGCTCATGCCTGTAATCCCAGCACTTTG
GGAGGCTAAGGTGGGTGGATCACCTGAGGTCAGGAGTTCGAGACAAGCCTAGCC
AACATAGTGAAACCCCGTCTCTACTAATAATACAAAAATTAGCTGGGTATGGCA
GCATGTGCCTGTAATCCCAGCTACTCGGGAGGCTGAGGCAGGAGAATTGCTCGA
ACCCGGGAGGCGGAGGTTGCAGTGAACCGAGAGAGATCGTGCGGTGCCATTTCA
CTCCAGCCTGGGCAACAGAGCGAAACTCCATCTCAAAAAAACACACAAAACAAA
CAAACAAAAAGAAAGAACCATTGTATTAGTGATGGAAATGTGTTCCCTCCCTCCC
ATCCTGGCAACCACTTTCTTCCTCCTCCATCATAAAATATCTTAAACTAAACTAA
AATAATTTTATTTATCGATAGTTTGAATTTTCCCTATCATTGCTACACAGCTAATT
GAGAGGTACCCCGAGGAAAATATAAATGGTACAGTAATGCATTGTAGATTTTAA
TAACATACTTGACATCCCAAATTGTTTTCATTGGCTTCATTTTAAAAACTACATGT
TTTAAAATCAAGCAGACACTAAAAGTACAAGATATACTGGGTCTACAAGGTTTA
AGTCAACCAGGGATTGAAATATAACTTTTAAACAGAGCTGGATTATCCAGTAGG
CAGATTAAGCATGTGCTTAAGGCATCAGCAAAGTCTGAGCAATCCATTTTTTAAA
ACGTAGTACATGTTTTTGATAAGCTTAAAAAGTAGTAGTCACAGGAAAAATTAG
AACTTTTACCTCCTTGCGCTTGTTATACTCTTTAGTGCTGTTTAACTTTTCTTTGTA
AGTGAGGGTGGTGGAGGGTGCCCATAATCTTTTCAGGGAGTAAGTTCTTCTTGGT
CTTTCTTTCTTTCTTTCTTTCTTTTTTTCTTGAGACCAAGTTTCGCTCTTGTCTCCCA
GGCTGGAGTGCAATGGCGCGATCTCGGCTCACTGCAACCTCCGCCTTCTCCTGGG
TTCAAGCGATTCTCCTACATCAGCCTCCGAGTAGCTGGGATTACAGGCATGCGCC
ACCAAGCCCCGCTAATTTTGTATTTTTTAGTAGAGACAGGGTTTCGCCATGTTGGT
CAGGCTTGTCTCGAACTCCTGGCCTCAGGTGATCCGCCTGTCTCGGCCTCCCAGA
ATGCTGGGATTATAGACGTGAGCCACCGCATCCGGACTTTCCTTTTATGTAATAG
TGATAATTCTATCCAAAGCATTTTTTTTTTTTTTTTTGAGTCGGAGTCTCATTCTGT
CACCCAGGCTGGAGGGTGGTGGCGCGATCTCGGCTTACTGCAACCTCTGCCTCCC
GGGTTCAAGCGATTCTCCTGCCTCAGCCTCCTGAGTAGCTGGAATTACACACGTG
CGCCACCATGGCCAGCTAATTTTTGTATTTTTAGTAGAGACGGGGTGTCACCATT
TTGGCCAAGCTGGCCTCGAACTCCTGACCTCAGGTGATCTGCCCGCCTCGGCTTC
CCAAAGTGCTGGGATTACAGGTGTGAGCCACCGCGTCCTGCTCCAAAGCATTTTC
TTTCTATGCCTCAAAACAAGATTGCAAGCCAGTCCTCAAAGCGGATAATTCAAGA
GCTAACAGGTATTAGCTTAGGATGTGTGGCACTGTTCTTAAGGCTTATATGTATT
AATACATCATTTAAACTCACAACAACCCCTATAAAGCAGGGGGCACTCATATTCC
CTTCCCCCTTTATAATTACGAAAAATGCAAGGTATTTTCAGTAGGAAAGAGAAAT
GTGAGAAGTGTGAAGGAGACAGGACAGTATTTGAAGCTGGTCTTTGGATCACTG
TGCAACTCTGCTTCTAGAACACTGAGCACTTTTTCTGGTCTAGGAATTATGACTTT
GAGAATGGAGTCCGTCCTTCCAATGACTCCCTCCCCATTTTCCTATCTGCCTACAG
GCAGAATTCTCCCCCGTCCGTATTAAATAAACCTCATCTTTTCAGAGTCTGCTCTT
ATACCAGGCAATGTACACGTCTGAGAAACCCTTGCCCCAGACAGCCGTTTTACAC
GCAGGAGGGGAAGGGGAGGGGAAGGAGAGAGCAGTCCGACTCTCCAAAAGGAA
TCCTTTGAACTAGGGTTTCTGACTTAGTGAACCCCGCGCTCCTGAAAATCAAGGG
TTGAGGGGGTAGGGGGACACTTTCTAGTCGTACAGGTGATTTCGATTCTCGGTGG
GGCTCTCACAACTAGGAAAGAATAGTTTTGCTTTTTCTTATGATTAAAAGAAGAA
GCCATACTTTCCCTATGACACCAAACACCCCGATTCAATTTGGCAGTTAGGAAGG
TTGTATCGCGGAGGAAGGAAACGGGGCGGGGGCGGATTTCTTTTTAACAGAGTG
AACGCACTCAAACACGCCTTTGCTGGCAGGCGGGGGAGCGCGGCTGGGAGCAGG
GAGGCCGGAGGGCGGTGTGGGGGGCAGGTGGGGAGGAGCCCAGTCCTCCTTCCT
TGCCAACGCTGGCTCTGGCGAGGGCTGCTTCCGGCTGGTGCCCCCGGGGGAGAC
CCAACCTGGGGCGACTTCAGGGGTGCCACATTCGCTAAGTGCTCGGAGTTAATA
GCACCTCCTCCGAGCACTCGCTCACGGCGTCCCCTTGCCTGGAAAGATACCGCGG
TCCCTCCAGAGGATTTGAGGGACAGGGTCGGAGGGGGCTCTTCCGCCAGCACCG
GAGGAAGAAAGAGGAGGGGCTGGCTGGTCACCAGAGGGTGGGGCGGACCGCGT
GCGCTCGGCGGCTGCGGAGAGGGGGAGAGCAGGCAGCGGGCGGCGGGGAGCAG
CATGGAGCCGGCGGCGGGGAGCAGCATGGAGCCTTCGGCTGACTGGCTGGCCAC
GGCCGCGGCCCGGGGTCGGGTAGAGGAGGTGCGGGCGCTGCTGGAGGCGGGGG
CGCTGCCCAACGCACCGAATAGTTACGGTCGGAGGCCGATCCAGGTGGGTAGAG
GGTCTGCAGCGGGAGCAGGGGATGGCGGGCGACTCTGGAGGACGAAGTTTGCAG
GGGAATTGGAATCAGGTAGCGCTTCGATTCTCCGGAAAAAGGGGAGGCTTCCTG
GGGAGTTTTCAGAAGGGGTTTGTAATCACAGACCTCCTCCTGGCGACGCCCTGGG
GGCTTGGGAAGCCAAGGAAGAGGAATGAGGAGCCACGCGCGTACAGATCTCTCG
AATGCTGAGAAGATCTGAAGGGGGGAACATATTTGTATTAGATGGAAGTATGC.

The EF1α promoter sequence (204) is a component of the DNA immortalization construct (200) responsible for driving the expression of the hTERT gene. This promoter, derived from the human elongation factor 1 alpha gene, is known for its strong and constitutive activity in a wide range of cell types. The EF1α promoter (204) ensures robust and consistent transcription of the hTERT gene, leading to high levels of telomerase expression and efficient cell immortalization. Conventional immortalization methods often rely on viral promoters or weaker endogenous promoters, resulting in variable or insufficient levels of gene expression. The EF1α promoter (204), by providing strong and consistent activity, overcomes this limitation and enhances the reliability of the immortalization process. Variations in the EF1α promoter sequence are possible, such as the use of truncated or modified versions, but the core promoter region must be retained to maintain its activity. Alternative promoters, such as CMV, CAG, or PGK, could be employed, but EF1α is generally preferred due to its broad applicability and consistent expression levels across different cell types. The EF1α promoter can be used to drive hTERT expression in human primary keratinocytes. The expression levels of hTERT driven by EF1α versus other promoters have increased and more consistent hTERT expression with EF1a. The strong and constitutive activity of the EF1α promoter offers a significant technical advantage over conventional methods for cell immortalization by ensuring high levels of hTERT expression and increasing the efficiency of telomere maintenance. This robust hTERT expression extends the replicative lifespan of primary cells and generating stable, immortalized cell lines.

In an embodiment, the EF1 promoter includes a nucleic acid base sequence comprising:

(SEQ. ID NO. 2)
CGTGAGGCTCCGGTGCCCGTCAGTGGGCAGAGCGCACATCGCCCACAGTCCCCG
AGAAGTTGGGGGGAGGGGTCGGCAATTGAACCGGTGCCTAGAGAAGGTGGCGC
GGGGTAAACTGGGAAAGTGATGTCGTGTACTGGCTCCGCCTTTTTCCCGAGGGTG
GGGGAGAACCGTATATAAGTGCAGTAGTCGCCGTGAACGTTCTTTTTCGCAACGG
GTTTGCCGCCAGAACACAGGTAAGTGCCGTGTGTGGTTCCCGCGGGCCTGGCCTC
TTTACGGGTTATGGCCCTTGCGTGCCTTGAATTACTTCCACGCCCCTGGCTGCAGT
ACGTGATTCTTGATCCCGAGCTTCGGGTTGGAAGTGGGTGGGAGAGTTCGAGGC
CTTGCGCTTAAGGAGCCCCTTCGCCTCGTGCTTGAGTTGAGGCCTGGCCTGGGCG
CTGGGGCCGCCGCGTGCGAATCTGGTGGCACCTTCGCGCCTGTCTCGCTGCTTTC
GATAAGTCTCTAGCCATTTAAAATTTTTGATGACCTGCTGCGACGCTTTTTTTCTG
GCAAGATAGTCTTGTAAATGCGGGCCAAGATCTGCACACTGGTATTTCGGTTTTT
GGGGCCGCGGGCGGCGACGGGGCCCGTGCGTCCCAGCGCACATGTTCGGCGAGG
CGGGGCCTGCGAGCGCGGCCACCGAGAATCGGACGGGGGTAGTCTCAAGCTGGC
CGGCCTGCTCTGGTGCCTGGCCTCGCGCCGCCGTGTATCGCCCCGCCCTGGGCGG
CAAGGCTGGCCCGGTCGGCACCAGTTGCGTGAGCGGAAAGATGGCCGCTTCCCG
GCCCTGCTGCAGGGAGCTCAAAATGGAGGACGCGGCGCTCGGGAGAGCGGGCG
GGTGAGTCACCCACACAAAGGAAAAGGGCCTTTCCGTCCTCAGCCGTCGCTTCAT
GTGACTCCACGGAGTACCGGGCGCCGTCCAGGCACCTCGATTAGTTCTCGAGCTT
TTGGAGTACGTCGTCTTTAGGTTGGGGGGAGGGGTTTTATGCGATGGAGTTTCCC
CACACTGAGTGGGTGGAGACTGAAGTTAGGCCAGCTTGGCACTTGATGTAATTCT
CCTTGGAATTTGCCCTTTTTGAGTTTGGATCTTGGTTCATTCTCAAGCCTCAGACA
GTGGTTCAAAGTTTTTTTCTTCCATTTCAGGTGTCGTGAGAATTC.

The first LoxP sequence (206), arranged upstream of the hTERT gene (208) in the sequence of DNA immortalization construct 200, is a component of the DNA immortalization construct's (200) design, enabling conditional regulation of hTERT expression. LoxP sequences are 34-base pair DNA sequences recognized by the Cre recombinase enzyme. When two LoxP sites are present in the same orientation on a DNA molecule, Cre recombinase catalyzes the excision of the intervening DNA segment. In the DNA immortalization construct (200), the first LoxP sequence (206), in conjunction with a second LoxP sequence (210) located downstream of hTERT (208), creates a removable hTERT expression cassette. This conditional control over hTERT expression provides significant flexibility for manipulation. By introducing Cre recombinase into the immortalized cells, the hTERT gene can be excised, reverting the cells to a non-immortalized state with a finite replicative lifespan. This feature is particularly valuable for studies of cellular differentiation, where hTERT expression may interfere with normal developmental processes, or for generating cell lines with controlled senescence for aging research. Variations in the placement of the LoxP sequence (206) relative to the hTERT gene are possible, but the sequence must be positioned to ensure efficient Cre-mediated recombination. Alternative recombination systems, such as Flp/FRT, could be employed, but the LoxP/Cre system is widely used and readily available, offering a convenient and well-characterized approach. This conditional control over hTERT expression, provided by the inclusion of LoxP sites in the DNA immortalization construct (200), represents a technical advantage over conventional immortalization methods that lack such precise regulatory mechanisms. This flexibility allows researchers to tailor hTERT expression to their specific experimental needs, generating cell lines with either extended or controlled lifespans.

In an embodiment, the first LoxP sequence includes a nucleic acid base sequence comprising:

(SEQ. ID NO. 3)
ATAACTTCGTATAGCATACATTATACGAAGTTAT.

The human TERT gene sequence (208) is a functional element of the DNA immortalization construct (200) responsible for extending the replicative lifespan of primary cells. This gene encodes the catalytic subunit of telomerase, a ribonucleoprotein enzyme involved for maintaining telomere length. Telomeres, repetitive DNA sequences at the ends of linear chromosomes, protect against genomic instability and degradation. With each cell division, telomeres shorten, eventually triggering cellular senescence and limiting the replicative capacity of primary cells. The introduction of the hTERT gene (208) provides cells with the enzymatic machinery to counteract telomere shortening, enabling them to bypass senescence and achieve continuous cell division. This effectively immortalizes the cell line, providing a consistent and readily available source of cells for research, development, and commercial applications. Variations in the hTERT gene sequence (208) are possible, including the use of codon-optimized versions for enhanced expression in specific cell types or the inclusion of tags or mutations for research purposes. However, the core catalytic domain of hTERT must be preserved for telomerase activity and cell immortalization. Alternative approaches to overcoming cellular senescence exist, such as inhibiting telomere-associated proteins, but direct hTERT expression offers a more direct and robust mechanism for telomere maintenance. The inclusion of the hTERT gene (208) provides a technical advantage in immortalization by directly addressing the fundamental mechanism of telomere shortening. This offers a more efficient approach compared with other strategies that target downstream components of the senescence pathway. The resulting immortalized cell lines provide a consistent and reliable source of material for research.

In an embodiment, hTERT gene DNA includes a nucleic acid base sequence comprising:

(SEQ. ID NO. 4)
ACCCCCGCGATGCCGCGCGCTCCCCGCTGCCGAGCCGTGCGCTCCCTGCTGCGCA
GCCACTACCGCGAGGTGCTGCCGCTGGCCACGTTCGTGCGGCGCCTGGGGCCCC
AGGGCTGGCGGCTGGTGCAGCGCGGGGACCCGGCGGCTTTCCGCGCGCTGGTGG
CCCAGTGCCTGGTGTGCGTGCCCTGGGACGCACGGCCGCCCCCCGCCGCCCCCTC
CTTCCGCCAGGTGTCCTGCCTGAAGGAGCTGGTGGCCCGAGTGCTGCAGAGGCT
GTGCGAGCGCGGCGCGAAGAACGTGCTGGCCTTCGGCTTCGCGCTGCTGGACGG
GGCCCGCGGGGGCCCCCCCGAGGCCTTCACCACCAGCGTGCGCAGCTACCTGCC
CAACACGGTGACCGACGCACTGCGGGGGAGCGGGGCGTGGGGGCTGCTGCTGCG
CCGCGTGGGCGACGACGTGCTGGTTCACCTGCTGGCACGCTGCGCGCTCTTTGTG
CTGGTGGCTCCCAGCTGCGCCTACCAGGTGTGCGGGCCGCCGCTGTACCAGCTCG
GCGCTGCCACTCAGGCCCGGCCCCCGCCACACGCTAGTGGACCCCGAAGGCGTC
TGGGATGCGAACGGGCCTGGAACCATAGCGTCAGGGAGGCCGGGGTCCCCCTGG
GCCTGCCAGCCCCGGGTGCGAGGAGGCGCGGGGGCAGTGCCAGCCGAAGTCTGC
CGTTGCCCAAGAGGCCCAGGCGTGGCGCTGCCCCTGAGCCGGAGCGGACGCCCG
TTGGGCAGGGGTCCTGGGCCCACCCGGGCAGGACGCGTGGACCGAGTGACCGTG
GTTTCTGTGTGGTGTCACCTGCCAGACCCGCCGAAGAAGCCACCTCTTTGGAGGG
TGCGCTCTCTGGCACGCGCCACTCCCACCCATCCGTGGGCCGCCAGCACCACGCG
GGCCCCCCATCCACATCGCGGCCACCACGTCCCTGGGACACGCCTTGTCCCCCGG
TGTACGCCGAGACCAAGCACTTCCTCTACTCCTCAGGCGACAAGGAGCAGCTGC
GGCCCTCCTTCCTACTCAGCTCTCTGAGGCCCAGCCTGACTGGCGCTCGGAGGCT
CGTGGAGACCATCTTTCTGGGTTCCAGGCCCTGGATGCCAGGGACTCCCCGCAGG
TTGCCCCGCCTGCCCCAGCGCTACTGGCAAATGCGGCCCCTGTTTCTGGAGCTGC
TTGGGAACCACGCGCAGTGCCCCTACGGGGTGCTCCTCAAGACGCACTGCCCGCT
GCGAGCTGCGGTCACCCCAGCAGCCGGTGTCTGTGCCCGGGAGAAGCCCCAGGG
CTCTGTGGCGGCCCCCGAGGAGGAGGACACAGACCCCCGTCGCCTGGTGCAGCT
GCTCCGCCAGCACAGCAGCCCCTGGCAGGTGTACGGCTTCGTGCGGGCCTGCCTG
CGCCGGCTGGTGCCCCCAGGCCTCTGGGGCTCCAGGCACAACGAACGCCGCTTC
CTCAGGAACACCAAGAAGTTCATCTCCCTGGGGAAGCATGCCAAGCTCTCGCTG
CAGGAGCTGACGTGGAAGATGAGCGTGCGGGACTGCGCTTGGCTGCGCAGGAGC
CCAGGGGTTGGCTGTGTTCCGGCCGCAGAGCACCGTCTGCGTGAGGAGATCCTG
GCCAAGTTCCTGCACTGGCTGATGAGTGTGTACGTCGTCGAGCTGCTCAGGTCTT
TCTTTTATGTCACGGAGACCACGTTTCAAAAGAACAGGCTCTTTTTCTACCGGAA
GAGTGTCTGGAGCAAGTTGCAAAGCATTGGAATCAGACAGCACTTGAAGAGGGT
GCAGCTGCGGGAGCTGTCGGAAGCAGAGGTCAGGCAGCATCGGGAAGCCAGGC
CCGCCCTGCTGACGTCCAGACTCCGCTTCATCCCCAAGCCTGACGGGCTGCGGCC
GATTGTGAACATGGACTACGTCGTGGGAGCCAGAACGTTCCGCAGAGAAAAGAG
GGCCGAGCGTCTCACCTCGAGGGTGAAGGCACTGTTCAGCGTGCTCAACTACGA
GCGGGCGCGGCGCCCCGGCCTCCTGGGCGCCTCTGTGCTGGGCCTGGACGATATC
CACAGGGCCTGGCGCACCTTCGTGCTGCGTGTGCGGGCCCAGGACCCGCCGCCT
GAGCTGTACTTTGTCAAGGTGGATGTGACGGGCGCGTACGACACCATCCCCCAG
GACAGGCTCACGGAGGTCATCGCCAGCATCATCAAACCCCAGAACACGTACTGC
GTGCGTCGGTATGCCGTGGTCCAGAAGGCCGCCCATGGGCACGTCCGCAAGGCC
TTCAAGAGCCACGTCTCTACCTTGACAGACCTCCAGCCGTACATGCGACAGTTCG
TGGCTCACCTGCAGGAGACCAGCCCGCTGAGGGATGCCGTCGTCATCGAGCAGA
GCTCCTCCCTGAATGAGGCCAGCAGTGGCCTCTTCGACGTCTTCCTACGCTTCAT
GTGCCACCACGCCGTGCGCATCAGGGGCAAGTCCTACGTCCAGTGCCAGGGGAT
CCCGCAGGGCTCCATCCTCTCCACGCTGCTCTGCAGCCTGTGCTACGGCGACATG
GAGAACAAGCTGTTTGCGGGGATTCGGCGGGACGGGCTGCTCCTGCGTTTGGTG
GATGATTTCTTGTTGGTGACACCTCACCTCACCCACGCGAAAACCTTCCTCAGGA
CCCTGGTCCGAGGTGTCCCTGAGTATGGCTGCGTGGTGAACTTGCGGAAGACAGT
GGTGAACTTCCCTGTAGAAGACGAGGCCCTGGGTGGCACGGCTTTTGTTCAGATG
CCGGCCCACGGCCTATTCCCCTGGTGCGGCCTGCTGCTGGATACCCGGACCCTGG
AGGTGCAGAGCGACTACTCCAGCTATGCCCGGACCTCCATCAGAGCCAGTCTCA
CCTTCAACCGCGGCTTCAAGGCTGGGAGGAACATGCGTCGCAAACTCTTTGGGGT
CTTGCGGCTGAAGTGTCACAGCCTGTTTCTGGATTTGCAGGTGAACAGCCTCCAG
ACGGTGTGCACCAACATCTACAAGATCCTCCTGCTGCAGGCGTACAGGTTTCACG
CATGTGTGCTGCAGCTCCCATTTCATCAGCAAGTTTGGAAGAACCCCACATTTTT
CCTGCGCGTCATCTCTGACACGGCCTCCCTCTGCTACTCCATCCTGAAAGCCAAG
AACGCAGGGATGTCGCTGGGGGCCAAGGGCGCCGCCGGCCCTCTGCCCTCCGAG
GCCGTGCAGTGGCTGTGCCACCAAGCATTCCTGCTCAAGCTGACTCGACACCGTG
TCACCTACGTGCCACTCCTGGGGTCACTCAGGACAGCCCAGACGCAGCTGAGTC
GGAAGCTCCCGGGGACGACGCTGACTGCCCTGGAGGCCGCAGCCAACCCGGCAC
TGCCCTCAGACTTCAAGACCATCCTGGACTGATGACCAAGCTT.

The second LoxP sequence (210), positioned downstream of the hTERT gene (208), completes the hTERT expression cassette flanked by LoxP sites. This arrangement is for the conditional regulation of hTERT expression, a feature of the DNA immortalization construct (200). The second LoxP sequence (210), which can be identical to the first LoxP sequence (206), provides the second recognition site for Cre recombinase. When Cre recombinase is introduced into cells containing the construct, it catalyzes recombination between the two LoxP sites, excising the intervening DNA, including the hTERT gene, from the genome. This controlled removal of the hTERT gene allows reversion of immortalized cells to a non-immortalized state or for study of specific effects of hTERT expression on cellular function. This precise control over gene expression is not readily achievable with conventional immortalization methods. Variations in the placement of the second LoxP sequence (210) are possible, but its position and orientation relative to the first LoxP site and the hTERT gene are involved for efficient Cre-mediated recombination. The inclusion of the second LoxP sequence (210) further enhances the versatility of the DNA immortalization construct (200), allowing for precise control of hTERT expression and facilitating a wide range of research applications.

In an embodiment, the second LoxP sequence includes the nucleic acid base sequence comprising SEQ. ID NO. 3.

The IRES sequence (212), located downstream of the second LoxP sequence (210), plays a role in the co-expression of the hTERT gene (208) and the antibiotic resistance gene sequence (214). IRES, or internal ribosomal entry site, is a specific RNA sequence that allows for cap-independent translation initiation. In the DNA immortalization construct (200), the IRES sequence (212) enables the production of two separate proteins, hTERT and the antibiotic resistance protein, from a single mRNA transcript. This co-expression strategy simplifies the construct design and ensures that all cells expressing hTERT also express the selectable marker, facilitating efficient selection and isolation of immortalized cells. Conventional approaches to co-expressing multiple genes often involve using separate promoters for each gene, which can increase the size and complexity of the construct and may lead to variable expression levels. The IRES sequence (212) offers a more compact and efficient solution, ensuring stoichiometric expression of both genes from a single promoter. Variations in the specific IRES sequence (212) used are possible, as different IRES elements exhibit varying degrees of translational efficiency. Alternative strategies for co-expression, such as using 2A peptides or fusion proteins, could be employed, but IRES sequences are widely used and well-characterized. The IRES sequence (212) offers a technical advantage by enabling the efficient co-expression of two involved genes, hTERT and the antibiotic resistance gene, from a single promoter. This simplifies construct design and ensures reliable selection of immortalized cell lines.

In an embodiment, the IRES includes a nucleic acid base sequence comprising:

(SEQ. ID NO. 5)
CCCCTCTCCCTCCCCCCCCCCTAACGTTACTGGCCGAAGCCGCTTGGAAT
AAGGCCGGTGTGCGTTTGTCTATATGTTATTTTCCACCATATTGCCGTCT
TTTGGCAATGTGAGGGCCCGGAAACCTGGCCCTGTCTTCTTGACGAGCAT
TCCTAGGGGTCTTTCCCCTCTCGCCAAAGGAATGCAAGGTCTGTTGAATG
TCGTGAAGGAAGCAGTTCCTCTGGAAGCTTCTTGAAGACAAACAACGTCT
GTAGCGACCCTTTGCAGGCAGCGGAACCCCCCACCTGGCAACAGGTGCCT
CTGCGGCCAAAAGCCACGTGTATAAGATACACCTGCAAAGGCGGCACAAC
CCCAGTGCCACGTTGTGAGTTGGATAGTTGTGGAAAGAGTCAAATGGCTC
TCCTCAAGCGTATTCAACAAGGGGCTGAAGGATGCCCAGAAGGTACCCCA
TTGTATGGGATCTGATCTGGGGCCTCGGTGCACATGCTTTACATGTGTTT
AGTCGAGGTTAAAAAAACGTCTAGGCCCCCCGAACCACGGGGACGTGGTT
TTCCTTTGAAAAACACGATGATAATA.

The antibiotic resistance gene sequence (214), linked to the IRES sequence (212), is for selecting and isolating cells that have successfully integrated the DNA immortalization construct (200). This gene confers resistance to a specific antibiotic, allowing transfected cells to survive in a selective medium containing the antibiotic, while non-transfected cells are eliminated. This selection process is for establishing pure populations of immortalized cells. The choice of antibiotic resistance gene (214) depends on the cell type and the available selection methods, and commonly used genes include those conferring resistance to neomycin, puromycin, hygromycin, or Zeocin. Variations in the specific antibiotic resistance gene sequence are possible, with different genes exhibiting varying degrees of resistance and compatibility with different cell types. For instance, a neomycin resistance gene might be preferred for certain cell types, while a puromycin resistance gene might be more suitable for others. Alternative selection strategies, such as fluorescent markers or cell surface antigens, could be employed, but antibiotic selection remains a widely used and efficient method for isolating transfected cells. This selection strategy provides a technical advantage by enabling researchers to easily isolate and expand populations of cells that have successfully integrated the DNA immortalization construct, thereby streamlining the process of establishing immortalized cell lines. The use of a specific selectable marker, coupled with the IRES-mediated co-expression of hTERT, ensures that all selected cells also carry the hTERT gene, involved for cell immortalization.

In an embodiment, the antibiotic resistance gene includes a nucleic acid base sequence comprising:

(SEQ. ID NO. 6)
GACCTGCAGCCCAAGCTTACCATGGCCAAGTTGACCAGTGCCGTTCCGGT
GCTCACCGCGCGCGACGTCGCCGGAGCGGTCGAGTTCTGGACCGACCGGC
TCGGGTTCTCCCGGGACTTCGTGGAGGACGACTTCGCCGGTGTGGTCCGG
GACGACGTGACCCTGTTCATCAGCGCGGTCCAGGACCAGGTGGTGCCGGA
CAACACCCTGGCCTGGGTGTGGGTGCGCGGCCTGGACGAGCTGTACGCCG
AGTGGTCGGAGGTCGTGTCCACGAACTTCCGGGACGCCTCCGGGCCGGCC
ATGACCGAGATCGGCGAGCAGCCGTGGGGGCGGGAGTTCGCCCTGCGCGA
CCCGGCCGGCAACTGCGTGCACTTCGTGGCCGAGGAGCAGGACTGAATCG
A.

The SV40 poly-A signal sequence (216), located downstream of the antibiotic resistance gene sequence (214), plays a role in enhancing the expression of both the hTERT gene (208) and the antibiotic resistance gene. A poly-A signal sequence directs the addition of a poly-A tail, a string of adenine nucleotides, to the 3′ end of mRNA transcripts. This poly-A tail is involved for mRNA stability, protecting the transcript from degradation and enhancing its translation efficiency. The SV40 poly-A signal (216), derived from the simian virus 40, is a commonly used and highly effective polyadenylation signal. By enhancing the stability and translation of the mRNA transcripts encoding hTERT and the antibiotic resistance protein, the SV40 poly-A signal (216) increases the levels of these proteins in transfected cells. This increased protein expression leads to more efficient cell immortalization and more robust antibiotic selection. Variations in the specific poly-A signal sequence (216) are possible, as different polyadenylation signals exhibit varying degrees of activity in different cell types. Alternative poly-A signals, such as those derived from bovine growth hormone or rabbit beta-globin genes, could be employed, but the SV40 poly-A signal is widely used and effective. The SV40 poly-A signal (216), by increasing mRNA stability and translation efficiency, contributes to the overall goal of generating stable, functionally representative immortalized cell lines.

In an embodiment, the SV40 PolyA includes a nucleic acid base sequence comprising:

(SEQ. ID NO. 7)
AACTTGTTTATTGCAGCTTATAATGGTTACAAATAAAGCAATAGCATCAC
AAATTTCACAAATAAAGCATTTTTTTCACTGCATTCTAGTTGTGGTTTGT
CCAAACTCATCAATGTATCTT.

The 3′ arm (218) of the DNA immortalization construct (200), derived from the 3′ end of the human CDKN2A gene exon 2, provides a second region of homology for targeted integration at the CDKN2A locus. Similar to the 5′ arm (202), the 3′ arm (218) facilitates homologous recombination by hybridizing with the genomic DNA at the target site. This second arm enhances the efficiency and precision of gene insertion, as homologous recombination involves regions of DNA identity on both sides of the inserted sequence. The inclusion of both a 5′ and a 3′ arm increases the likelihood of correct integration and minimizes the risk of random insertion events, which can disrupt gene function or lead to genomic instability. Variations in the length of the 3′ arm (218) are possible, with sequences ranging from a few hundred base pairs to several kilobases. Longer arms typically offer increased targeting specificity but can be more difficult to synthesize or clone. Alternative sequences derived from other regions of the CDKN2A gene, or from different genes altogether, could be used, but sequences flanking exon 2 are preferred for their ability to disrupt the CDKN2A gene while simultaneously introducing the hTERT expression cassette. The 3′ arm's role in facilitating targeted homologous recombination offers a technical advantage in cell immortalization by ensuring precise gene insertion and minimizing the risk of off-target effects. This precise targeting capability enhances genomic stability and contributes to the production of immortalized cell lines that faithfully represent their primary cell counterparts.

In an embodiment, the 3′ homologous arm includes a nucleic acid base sequence comprising:

(SEQ. ID NO. 8)
CTCTAAGCCCTCTGCTTGGAGATGCTGTAAATACAGAACGCAAAATCACCTTCGA
AGTTAAAGACGCGAAGTTCTTCTTTACTCGGCCCCTCCTCCCCTCCCCCCCGCCA
ATTCCCTCCAGTTACAGCTAGCATCCAGGTCCCGGGAGGTGAAGAAGGAGACTT
CGGCTCCAGTTACAGCTAGCATCCGGGTCCCGATTTAGAAGGAGCTGCCAATTAC
AGCGCGGTTCCAGGGCTGAGCAAAAAGCCTGAGGAGCCAAGTGGGAGAGGGAG
TAAAACTACTGAATTGGGCCACAAGCAAATGAATAAACTGAACGACTCTTAACC
AAACCTAATATATTTAATCCAAACACACAAGTCTTTCATTTCTTCCCTCCTCCCTT
CCTTCTCTTACTCCCCAACACCCCCTCTTCAAGCACAATTAATTATATGGTTAGAT
TCTACTGCGTGATCAGCCCTGTTCTAGGTGGTGGGCACGCCAAGGTGAATGAGAC
CAAACAAGAGTCTTGCCCTCATGGGGTTTACATTTGGAGACAGAGTCGATCTGTT
GCCCAACCTGGAGTGCAGTGGCGCGATCACAGCTCACTGCAGCCTCAAACTCCCT
GGCTCAAGGGGTTCTCCCACCTGAGCCTCCCGACTAGCTGGGACCACAGGTGCA
CGCCACGACGCCTGGGTTTGTTTGTTTGTTTAATAGAGACGAAGGTCTCACCATG
TTATCTGGGCTCAAGCGATCATCCCCCCTCCTCCTCCTAAAGTACTGGGATTACA
GTCCCAAGCTATCTTGCCCGACCTGGGAAACAGACGTTAAGGAAGATAACAATC
TATTTTCAGAGAGCGAGTTTATAAAACCAATGCAATGGGTAAATATGAAGTGTG
AATAGGAGGAGAAGCTAAAGAGTGGTCGGAGAATCTAATGCAAGCTACGGGAG
AAAGAAACTCAAGTGCAAATGCTGCCTCAGGAATAAACGTAAAAAGAGACTTTC
AAGTGCAAATGCTCCCTCAGGAATAAAATAATCTTGAGACTCTCAAGTGTAAAT
GCTGCCTCGGGAGAACCGAACGGCGAGCTGGAGCCCATACGCAACGAGATTAGA
GAGGAAGGCAGAAGCCAGAGCACATGAATAAATGAGCATCCATTTTGTTTCAGA
AATGATCGGAAACCATTTGTGGGTTTGTAGAAGCAGGCATGCGTAGGGAAGCTA
CGGGATTCCGCCGAGGAGCGCCAGAGCCTGAGGCGCCCTTTGGTTATCGCAAGC
TGGCTGGCTCACTCCGCACCAGGTGCAAAAGATGCCTGGGGATGCGGGAAGGGA
AAGGCCACATCTTCACGCCTTCGCGCCTGGCATTGTGAGCAACCACTGAGACTCA
TTATATAACACTCGTTTTCTTCTTGCAACCCTGCGGGCCGCGCGGTCGCGCTTTCT
CTGCCCTCCGCCGGGTGGACCTGGAGCGCTTGAGCGGTCGGCGCGCCTGGAGCA
GCCAGGCGGGCAGTGGACTAGCTGCTGGACCAGGGAGGTGTGGGAGAGCGGTG
GCGGCGGGTACATGCACGTGAAGCCATTGCGAGAACTTTATCCATAAGTATTTCA
ATGCCGGTAGGGACGGCAAGAGAGGAGGGCGGGATGTGCCACACATCTTTGACC
TCAGGTTTCTAACGCCTGTTTTCTTTCTGCCCTCTGCAGACATCCCCGATTGAAAG
AACCAGAGAGGCTCTGAGAAACCTCGGGAAACTTAGATCATCAGTCACCGAAGG
TCCTACAGGGCCACAACTGCCCCCGCCACAACCCACCCCGCTTTCGTAGTTTTCA
TTTAGAAAATAGAGCTTTTAAAAATGTCCTGCCTTTTAACGTAGATATATGCCTTC
CCCCACTACCGTAAATGTCCATTTATATCATTTTTTATATATTCTTATAAAAATGT
AAAAAAGAAAAACACCGCTTCTGCCTTTTCACTGTGTTGGAGTTTTCTGGAGTGA
GCACTCACGCCCTAAGCGCACATTCATGTGGGCATTTCTTGCGAGCCTCGCAGCC
TCCGGAAGCTGTCGACTTCATGACAAGCATTTTGTGAACTAGGGAAGCTCAGGG
GGGTTACTGGCTTCTCTTGAGTCACACTGCTAGCAAATGGCAGAACCAAAGCTCA
AATAAAAATAAAATAATTTTCATTCATTCACTCA.

In an embodiment, the DNA immortalization construct includes a nucleic acid base sequence comprising:

(SEQ. ID NO. 9)
GATCCGGAATAAGTCTAAATGAATCACTTTCAGTTTTCCTAAACTTCTATGCCTTT
GCACATCCTCTTACCTCTGCCTAGAATATCTTTCTCCTTCTTTTCCATCTTTAAACT
CTCACATCATTCTTCAAGACTGGGATCAGCTCTCAGCATCCGGAAGCCTTTGCCT
ACTAGAGACAAATGAGAATGAGTTTGGTCACCTTTTCATTTTCTTGTATCATTCTG
TGCTTTATTTTGCTCTTCTAAGAGCGTTACATGCTTCATTTAATCCCTAAACAACT
GTTTGAGGCAAGTACAGTTATTATCCTAATCATGCAAATGAGAAAACAGAGGCC
CAGACATGTTGAGTAACTTTGATAAAAGTTAAAGAACCAATAAGTGGAACAGTT
GAGGTTTGAACCCTGGCAGTCTGACTGTAGAGATACTATGTTTGACCTACTCCCC
TCTGCCCCCACCCCATGTCTGCCCTTAGTTTCTGAGCTTGTTGAATGAATGAACA
GGTGGTAGTCTTTTTTTGTTATAAGACTGATCAGAATTAAGACAGGTTTAAATTTC
ACGTGTAGAATTTTCAAAACTGCAAAGGCAGTGCAAATCTAAAAAAAGAATGGC
ATTCTCAGGAAAGAGGAAAAGTAAGTGTGAGAATAATAATAACAATAACCAACA
AACTTTAGTAAATTTAGTAAATGTAGTAAATTTTTACATTAAAAGCTTTTGGACA
TACATTATCATATTTTATGGCCACATGAAATATATTATAATCCCATTTTGCACATA
GGAAATCTGAGACTGGCATAAGGAGCACAGAGATCCAGGACTTTATATTTTCATT
CTTCTAGGATTTTGCACCTCAGGTCGATATGTATGAGTAAACTGGGAGTATAATG
GGCTCTTTAACAGAAAAACTAGGAAAGTTTTCCCACTATTATTAATTATTTACAT
AATATTTTTTTAATTTTATTATTATTTATACTTTAAGTTTTAGAGTACATGTGCACA
ATGTGCAGGTTTGTTACATATGTATACATGTGCCATGTTGGTGTGCTGCACCCATC
AACTCATCATTTAGCATTAGGTATATCTCCTAATGCTATCCCTCCCCCCTCCCCCC
TACATAAGATTTATAATGGATAATGGACTTCAATTTCTAGAGCAAAATGGCCCCA
CCCAAGGATGCCATAATCCTTCCAGAGCTCTACTGCAAGATATGAGATATACATA
TCTAAAACTTGTTCTTGGTATTTCCAAAGCAGTCAACTTTTACACCTGTTTATAAT
GCATCCAAATGTTGTTTTTATATGGTTGCATCTCCCATCTTCTTCACCAATAGCTA
TATATATTTTTCACAAGAGCTGAAAGAGTTCTTGATGTAGGAATCCATGGTAGAG
TTTCAGAGAAATCCCTGAATTCACTGAAAGTTTTATCTAGAAATACATGTGCAAG
TGAACACATCTTTTTTAAAAAAAATCATTACCTACTTTCTTTTTTGAGAAGAAGGT
ATTTATTTCAACAGACTCTTGAAGGAGCCTACTCTTCCCACTCTCCCACCCCCATT
AAGAACCACTGTAGGCCGGGCACGATGGCTCATGCCTGTAATCCCAGCACTTTG
GGAGGCTAAGGTGGGTGGATCACCTGAGGTCAGGAGTTCGAGACAAGCCTAGCC
AACATAGTGAAACCCCGTCTCTACTAATAATACAAAAATTAGCTGGGTATGGCA
GCATGTGCCTGTAATCCCAGCTACTCGGGAGGCTGAGGCAGGAGAATTGCTCGA
ACCCGGGAGGCGGAGGTTGCAGTGAACCGAGAGAGATCGTGCGGTGCCATTTCA
CTCCAGCCTGGGCAACAGAGCGAAACTCCATCTCAAAAAAACACACAAAACAAA
CAAACAAAAAGAAAGAACCATTGTATTAGTGATGGAAATGTGTTCCCTCCCTCCC
ATCCTGGCAACCACTTTCTTCCTCCTCCATCATAAAATATCTTAAACTAAACTAA
AATAATTTTATTTATCGATAGTTTGAATTTTCCCTATCATTGCTACACAGCTAATT
GAGAGGTACCCCGAGGAAAATATAAATGGTACAGTAATGCATTGTAGATTTTAA
TAACATACTTGACATCCCAAATTGTTTTCATTGGCTTCATTTTAAAAACTACATGT
TTTAAAATCAAGCAGACACTAAAAGTACAAGATATACTGGGTCTACAAGGTTTA
AGTCAACCAGGGATTGAAATATAACTTTTAAACAGAGCTGGATTATCCAGTAGG
CAGATTAAGCATGTGCTTAAGGCATCAGCAAAGTCTGAGCAATCCATTTTTTAAA
ACGTAGTACATGTTTTTGATAAGCTTAAAAAGTAGTAGTCACAGGAAAAATTAG
AACTTTTACCTCCTTGCGCTTGTTATACTCTTTAGTGCTGTTTAACTTTTCTTTGTA
AGTGAGGGTGGTGGAGGGTGCCCATAATCTTTTCAGGGAGTAAGTTCTTCTTGGT
CTTTCTTTCTTTCTTTCTTTCTTTTTTTCTTGAGACCAAGTTTCGCTCTTGTCTCCCA
GGCTGGAGTGCAATGGCGCGATCTCGGCTCACTGCAACCTCCGCCTTCTCCTGGG
TTCAAGCGATTCTCCTACATCAGCCTCCGAGTAGCTGGGATTACAGGCATGCGCC
ACCAAGCCCCGCTAATTTTGTATTTTTTAGTAGAGACAGGGTTTCGCCATGTTGGT
CAGGCTTGTCTCGAACTCCTGGCCTCAGGTGATCCGCCTGTCTCGGCCTCCCAGA
ATGCTGGGATTATAGACGTGAGCCACCGCATCCGGACTTTCCTTTTATGTAATAG
TGATAATTCTATCCAAAGCATTTTTTTTTTTTTTTTTGAGTCGGAGTCTCATTCTGT
CACCCAGGCTGGAGGGTGGTGGCGCGATCTCGGCTTACTGCAACCTCTGCCTCCC
GGGTTCAAGCGATTCTCCTGCCTCAGCCTCCTGAGTAGCTGGAATTACACACGTG
CGCCACCATGGCCAGCTAATTTTTGTATTTTTAGTAGAGACGGGGTGTCACCATT
TTGGCCAAGCTGGCCTCGAACTCCTGACCTCAGGTGATCTGCCCGCCTCGGCTTC
CCAAAGTGCTGGGATTACAGGTGTGAGCCACCGCGTCCTGCTCCAAAGCATTTTC
TTTCTATGCCTCAAAACAAGATTGCAAGCCAGTCCTCAAAGCGGATAATTCAAGA
GCTAACAGGTATTAGCTTAGGATGTGTGGCACTGTTCTTAAGGCTTATATGTATT
AATACATCATTTAAACTCACAACAACCCCTATAAAGCAGGGGGCACTCATATTCC
CTTCCCCCTTTATAATTACGAAAAATGCAAGGTATTTTCAGTAGGAAAGAGAAAT
GTGAGAAGTGTGAAGGAGACAGGACAGTATTTGAAGCTGGTCTTTGGATCACTG
TGCAACTCTGCTTCTAGAACACTGAGCACTTTTTCTGGTCTAGGAATTATGACTTT
GAGAATGGAGTCCGTCCTTCCAATGACTCCCTCCCCATTTTCCTATCTGCCTACAG
GCAGAATTCTCCCCCGTCCGTATTAAATAAACCTCATCTTTTCAGAGTCTGCTCTT
ATACCAGGCAATGTACACGTCTGAGAAACCCTTGCCCCAGACAGCCGTTTTACAC
GCAGGAGGGGAAGGGGAGGGGAAGGAGAGAGCAGTCCGACTCTCCAAAAGGAA
TCCTTTGAACTAGGGTTTCTGACTTAGTGAACCCCGCGCTCCTGAAAATCAAGGG
TTGAGGGGGTAGGGGGACACTTTCTAGTCGTACAGGTGATTTCGATTCTCGGTGG
GGCTCTCACAACTAGGAAAGAATAGTTTTGCTTTTTCTTATGATTAAAAGAAGAA
GCCATACTTTCCCTATGACACCAAACACCCCGATTCAATTTGGCAGTTAGGAAGG
TTGTATCGCGGAGGAAGGAAACGGGGCGGGGGCGGATTTCTTTTTAACAGAGTG
AACGCACTCAAACACGCCTTTGCTGGCAGGCGGGGGAGCGCGGCTGGGAGCAGG
GAGGCCGGAGGGCGGTGTGGGGGGCAGGTGGGGAGGAGCCCAGTCCTCCTTCCT
TGCCAACGCTGGCTCTGGCGAGGGCTGCTTCCGGCTGGTGCCCCCGGGGGAGAC
CCAACCTGGGGCGACTTCAGGGGTGCCACATTCGCTAAGTGCTCGGAGTTAATA
GCACCTCCTCCGAGCACTCGCTCACGGCGTCCCCTTGCCTGGAAAGATACCGCGG
TCCCTCCAGAGGATTTGAGGGACAGGGTCGGAGGGGGCTCTTCCGCCAGCACCG
GAGGAAGAAAGAGGAGGGGCTGGCTGGTCACCAGAGGGTGGGGCGGACCGCGT
GCGCTCGGCGGCTGCGGAGAGGGGGAGAGCAGGCAGCGGGCGGCGGGGAGCAG
CATGGAGCCGGCGGCGGGGAGCAGCATGGAGCCTTCGGCTGACTGGCTGGCCAC
GGCCGCGGCCCGGGGTCGGGTAGAGGAGGTGCGGGCGCTGCTGGAGGCGGGGG
CGCTGCCCAACGCACCGAATAGTTACGGTCGGAGGCCGATCCAGGTGGGTAGAG
GGTCTGCAGCGGGAGCAGGGGATGGCGGGCGACTCTGGAGGACGAAGTTTGCAG
GGGAATTGGAATCAGGTAGCGCTTCGATTCTCCGGAAAAAGGGGAGGCTTCCTG
GGGAGTTTTCAGAAGGGGTTTGTAATCACAGACCTCCTCCTGGCGACGCCCTGGG
GGCTTGGGAAGCCAAGGAAGAGGAATGAGGAGCCACGCGCGTACAGATCTCTCG
AATGCTGAGAAGATCTGAAGGGGGGAACATATTTGTATTAGATGGAAGTATGCC
GTGAGGCTCCGGTGCCCGTCAGTGGGCAGAGCGCACATCGCCCACAGTCCCCGA
GAAGTTGGGGGGAGGGGTCGGCAATTGAACCGGTGCCTAGAGAAGGTGGCGCG
GGGTAAACTGGGAAAGTGATGTCGTGTACTGGCTCCGCCTTTTTCCCGAGGGTGG
GGGAGAACCGTATATAAGTGCAGTAGTCGCCGTGAACGTTCTTTTTCGCAACGGG
TTTGCCGCCAGAACACAGGTAAGTGCCGTGTGTGGTTCCCGCGGGCCTGGCCTCT
TTACGGGTTATGGCCCTTGCGTGCCTTGAATTACTTCCACGCCCCTGGCTGCAGT
ACGTGATTCTTGATCCCGAGCTTCGGGTTGGAAGTGGGTGGGAGAGTTCGAGGC
CTTGCGCTTAAGGAGCCCCTTCGCCTCGTGCTTGAGTTGAGGCCTGGCCTGGGCG
CTGGGGCCGCCGCGTGCGAATCTGGTGGCACCTTCGCGCCTGTCTCGCTGCTTTC
GATAAGTCTCTAGCCATTTAAAATTTTTGATGACCTGCTGCGACGCTTTTTTTCTG
GCAAGATAGTCTTGTAAATGCGGGCCAAGATCTGCACACTGGTATTTCGGTTTTT
GGGGCCGCGGGCGGCGACGGGGCCCGTGCGTCCCAGCGCACATGTTCGGCGAGG
CGGGGCCTGCGAGCGCGGCCACCGAGAATCGGACGGGGGTAGTCTCAAGCTGGC
CGGCCTGCTCTGGTGCCTGGCCTCGCGCCGCCGTGTATCGCCCCGCCCTGGGCGG
CAAGGCTGGCCCGGTCGGCACCAGTTGCGTGAGCGGAAAGATGGCCGCTTCCCG
GCCCTGCTGCAGGGAGCTCAAAATGGAGGACGCGGCGCTCGGGAGAGCGGGCG
GGTGAGTCACCCACACAAAGGAAAAGGGCCTTTCCGTCCTCAGCCGTCGCTTCAT
GTGACTCCACGGAGTACCGGGCGCCGTCCAGGCACCTCGATTAGTTCTCGAGCTT
TTGGAGTACGTCGTCTTTAGGTTGGGGGGAGGGGTTTTATGCGATGGAGTTTCCC
CACACTGAGTGGGTGGAGACTGAAGTTAGGCCAGCTTGGCACTTGATGTAATTCT
CCTTGGAATTTGCCCTTTTTGAGTTTGGATCTTGGTTCATTCTCAAGCCTCAGACA
GTGGTTCAAAGTTTTTTTCTTCCATTTCAGGTGTCGTGAGAATTCATAACTTCGTA
TAGCATACATTATACGAAGTTATACCCCCGCGATGCCGCGCGCTCCCCGCTGCCG
AGCCGTGCGCTCCCTGCTGCGCAGCCACTACCGCGAGGTGCTGCCGCTGGCCACG
TTCGTGCGGCGCCTGGGGCCCCAGGGCTGGCGGCTGGTGCAGCGCGGGGACCCG
GCGGCTTTCCGCGCGCTGGTGGCCCAGTGCCTGGTGTGCGTGCCCTGGGACGCAC
GGCCGCCCCCCGCCGCCCCCTCCTTCCGCCAGGTGTCCTGCCTGAAGGAGCTGGT
GGCCCGAGTGCTGCAGAGGCTGTGCGAGCGCGGCGCGAAGAACGTGCTGGCCTT
CGGCTTCGCGCTGCTGGACGGGGCCCGCGGGGGCCCCCCCGAGGCCTTCACCAC
CAGCGTGCGCAGCTACCTGCCCAACACGGTGACCGACGCACTGCGGGGGAGCGG
GGCGTGGGGGCTGCTGCTGCGCCGCGTGGGCGACGACGTGCTGGTTCACCTGCT
GGCACGCTGCGCGCTCTTTGTGCTGGTGGCTCCCAGCTGCGCCTACCAGGTGTGC
GGGCCGCCGCTGTACCAGCTCGGCGCTGCCACTCAGGCCCGGCCCCCGCCACAC
GCTAGTGGACCCCGAAGGCGTCTGGGATGCGAACGGGCCTGGAACCATAGCGTC
AGGGAGGCCGGGGTCCCCCTGGGCCTGCCAGCCCCGGGTGCGAGGAGGCGCGGG
GGCAGTGCCAGCCGAAGTCTGCCGTTGCCCAAGAGGCCCAGGCGTGGCGCTGCC
CCTGAGCCGGAGCGGACGCCCGTTGGGCAGGGGTCCTGGGCCCACCCGGGCAGG
ACGCGTGGACCGAGTGACCGTGGTTTCTGTGTGGTGTCACCTGCCAGACCCGCCG
AAGAAGCCACCTCTTTGGAGGGTGCGCTCTCTGGCACGCGCCACTCCCACCCATC
CGTGGGCCGCCAGCACCACGCGGGCCCCCCATCCACATCGCGGCCACCACGTCC
CTGGGACACGCCTTGTCCCCCGGTGTACGCCGAGACCAAGCACTTCCTCTACTCC
TCAGGCGACAAGGAGCAGCTGCGGCCCTCCTTCCTACTCAGCTCTCTGAGGCCCA
GCCTGACTGGCGCTCGGAGGCTCGTGGAGACCATCTTTCTGGGTTCCAGGCCCTG
GATGCCAGGGACTCCCCGCAGGTTGCCCCGCCTGCCCCAGCGCTACTGGCAAAT
GCGGCCCCTGTTTCTGGAGCTGCTTGGGAACCACGCGCAGTGCCCCTACGGGGTG
CTCCTCAAGACGCACTGCCCGCTGCGAGCTGCGGTCACCCCAGCAGCCGGTGTCT
GTGCCCGGGAGAAGCCCCAGGGCTCTGTGGCGGCCCCCGAGGAGGAGGACACA
GACCCCCGTCGCCTGGTGCAGCTGCTCCGCCAGCACAGCAGCCCCTGGCAGGTGT
ACGGCTTCGTGCGGGCCTGCCTGCGCCGGCTGGTGCCCCCAGGCCTCTGGGGCTC
CAGGCACAACGAACGCCGCTTCCTCAGGAACACCAAGAAGTTCATCTCCCTGGG
GAAGCATGCCAAGCTCTCGCTGCAGGAGCTGACGTGGAAGATGAGCGTGCGGGA
CTGCGCTTGGCTGCGCAGGAGCCCAGGGGTTGGCTGTGTTCCGGCCGCAGAGCA
CCGTCTGCGTGAGGAGATCCTGGCCAAGTTCCTGCACTGGCTGATGAGTGTGTAC
GTCGTCGAGCTGCTCAGGTCTTTCTTTTATGTCACGGAGACCACGTTTCAAAAGA
ACAGGCTCTTTTTCTACCGGAAGAGTGTCTGGAGCAAGTTGCAAAGCATTGGAAT
CAGACAGCACTTGAAGAGGGTGCAGCTGCGGGAGCTGTCGGAAGCAGAGGTCA
GGCAGCATCGGGAAGCCAGGCCCGCCCTGCTGACGTCCAGACTCCGCTTCATCCC
CAAGCCTGACGGGCTGCGGCCGATTGTGAACATGGACTACGTCGTGGGAGCCAG
AACGTTCCGCAGAGAAAAGAGGGCCGAGCGTCTCACCTCGAGGGTGAAGGCACT
GTTCAGCGTGCTCAACTACGAGCGGGCGCGGCGCCCCGGCCTCCTGGGCGCCTCT
GTGCTGGGCCTGGACGATATCCACAGGGCCTGGCGCACCTTCGTGCTGCGTGTGC
GGGCCCAGGACCCGCCGCCTGAGCTGTACTTTGTCAAGGTGGATGTGACGGGCG
CGTACGACACCATCCCCCAGGACAGGCTCACGGAGGTCATCGCCAGCATCATCA
AACCCCAGAACACGTACTGCGTGCGTCGGTATGCCGTGGTCCAGAAGGCCGCCC
ATGGGCACGTCCGCAAGGCCTTCAAGAGCCACGTCTCTACCTTGACAGACCTCCA
GCCGTACATGCGACAGTTCGTGGCTCACCTGCAGGAGACCAGCCCGCTGAGGGA
TGCCGTCGTCATCGAGCAGAGCTCCTCCCTGAATGAGGCCAGCAGTGGCCTCTTC
GACGTCTTCCTACGCTTCATGTGCCACCACGCCGTGCGCATCAGGGGCAAGTCCT
ACGTCCAGTGCCAGGGGATCCCGCAGGGCTCCATCCTCTCCACGCTGCTCTGCAG
CCTGTGCTACGGCGACATGGAGAACAAGCTGTTTGCGGGGATTCGGCGGGACGG
GCTGCTCCTGCGTTTGGTGGATGATTTCTTGTTGGTGACACCTCACCTCACCCACG
CGAAAACCTTCCTCAGGACCCTGGTCCGAGGTGTCCCTGAGTATGGCTGCGTGGT
GAACTTGCGGAAGACAGTGGTGAACTTCCCTGTAGAAGACGAGGCCCTGGGTGG
CACGGCTTTTGTTCAGATGCCGGCCCACGGCCTATTCCCCTGGTGCGGCCTGCTG
CTGGATACCCGGACCCTGGAGGTGCAGAGCGACTACTCCAGCTATGCCCGGACC
TCCATCAGAGCCAGTCTCACCTTCAACCGCGGCTTCAAGGCTGGGAGGAACATG
CGTCGCAAACTCTTTGGGGTCTTGCGGCTGAAGTGTCACAGCCTGTTTCTGGATT
TGCAGGTGAACAGCCTCCAGACGGTGTGCACCAACATCTACAAGATCCTCCTGCT
GCAGGCGTACAGGTTTCACGCATGTGTGCTGCAGCTCCCATTTCATCAGCAAGTT
TGGAAGAACCCCACATTTTTCCTGCGCGTCATCTCTGACACGGCCTCCCTCTGCT
ACTCCATCCTGAAAGCCAAGAACGCAGGGATGTCGCTGGGGGCCAAGGGCGCCG
CCGGCCCTCTGCCCTCCGAGGCCGTGCAGTGGCTGTGCCACCAAGCATTCCTGCT
CAAGCTGACTCGACACCGTGTCACCTACGTGCCACTCCTGGGGTCACTCAGGACA
GCCCAGACGCAGCTGAGTCGGAAGCTCCCGGGGACGACGCTGACTGCCCTGGAG
GCCGCAGCCAACCCGGCACTGCCCTCAGACTTCAAGACCATCCTGGACTGATGA
CCAAGCTTATAACTTCGTATAGCATACATTATACGAAGTTATCCCCTCTCCCTCCC
CCCCCCCTAACGTTACTGGCCGAAGCCGCTTGGAATAAGGCCGGTGTGCGTTTGT
CTATATGTTATTTTCCACCATATTGCCGTCTTTTGGCAATGTGAGGGCCCGGAAA
CCTGGCCCTGTCTTCTTGACGAGCATTCCTAGGGGTCTTTCCCCTCTCGCCAAAGG
AATGCAAGGTCTGTTGAATGTCGTGAAGGAAGCAGTTCCTCTGGAAGCTTCTTGA
AGACAAACAACGTCTGTAGCGACCCTTTGCAGGCAGCGGAACCCCCCACCTGGC
AACAGGTGCCTCTGCGGCCAAAAGCCACGTGTATAAGATACACCTGCAAAGGCG
GCACAACCCCAGTGCCACGTTGTGAGTTGGATAGTTGTGGAAAGAGTCAAATGG
CTCTCCTCAAGCGTATTCAACAAGGGGCTGAAGGATGCCCAGAAGGTACCCCAT
TGTATGGGATCTGATCTGGGGCCTCGGTGCACATGCTTTACATGTGTTTAGTCGA
GGTTAAAAAAACGTCTAGGCCCCCCGAACCACGGGGACGTGGTTTTCCTTTGAA
AAACACGATGATAATAGACCTGCAGCCCAAGCTTACCATGGCCAAGTTGACCAG
TGCCGTTCCGGTGCTCACCGCGCGCGACGTCGCCGGAGCGGTCGAGTTCTGGACC
GACCGGCTCGGGTTCTCCCGGGACTTCGTGGAGGACGACTTCGCCGGTGTGGTCC
GGGACGACGTGACCCTGTTCATCAGCGCGGTCCAGGACCAGGTGGTGCCGGACA
ACACCCTGGCCTGGGTGTGGGTGCGCGGCCTGGACGAGCTGTACGCCGAGTGGT
CGGAGGTCGTGTCCACGAACTTCCGGGACGCCTCCGGGCCGGCCATGACCGAGA
TCGGCGAGCAGCCGTGGGGGCGGGAGTTCGCCCTGCGCGACCCGGCCGGCAACT
GCGTGCACTTCGTGGCCGAGGAGCAGGACTGAATCGAAACTTGTTTATTGCAGCT
TATAATGGTTACAAATAAAGCAATAGCATCACAAATTTCACAAATAAAGCATTTT
TTTCACTGCATTCTAGTTGTGGTTTGTCCAAACTCATCAATGTATCTTACTCTAAG
CCCTCTGCTTGGAGATGCTGTAAATACAGAACGCAAAATCACCTTCGAAGTTAAA
GACGCGAAGTTCTTCTTTACTCGGCCCCTCCTCCCCTCCCCCCCGCCAATTCCCTC
CAGTTACAGCTAGCATCCAGGTCCCGGGAGGTGAAGAAGGAGACTTCGGCTCCA
GTTACAGCTAGCATCCGGGTCCCGATTTAGAAGGAGCTGCCAATTACAGCGCGG
TTCCAGGGCTGAGCAAAAAGCCTGAGGAGCCAAGTGGGAGAGGGAGTAAAACT
ACTGAATTGGGCCACAAGCAAATGAATAAACTGAACGACTCTTAACCAAACCTA
ATATATTTAATCCAAACACACAAGTCTTTCATTTCTTCCCTCCTCCCTTCCTTCTCT
TACTCCCCAACACCCCCTCTTCAAGCACAATTAATTATATGGTTAGATTCTACTGC
GTGATCAGCCCTGTTCTAGGTGGTGGGCACGCCAAGGTGAATGAGACCAAACAA
GAGTCTTGCCCTCATGGGGTTTACATTTGGAGACAGAGTCGATCTGTTGCCCAAC
CTGGAGTGCAGTGGCGCGATCACAGCTCACTGCAGCCTCAAACTCCCTGGCTCAA
GGGGTTCTCCCACCTGAGCCTCCCGACTAGCTGGGACCACAGGTGCACGCCACG
ACGCCTGGGTTTGTTTGTTTGTTTAATAGAGACGAAGGTCTCACCATGTTATCTGG
GCTCAAGCGATCATCCCCCCTCCTCCTCCTAAAGTACTGGGATTACAGTCCCAAG
CTATCTTGCCCGACCTGGGAAACAGACGTTAAGGAAGATAACAATCTATTTTCAG
AGAGCGAGTTTATAAAACCAATGCAATGGGTAAATATGAAGTGTGAATAGGAGG
AGAAGCTAAAGAGTGGTCGGAGAATCTAATGCAAGCTACGGGAGAAAGAAACT
CAAGTGCAAATGCTGCCTCAGGAATAAACGTAAAAAGAGACTTTCAAGTGCAAA
TGCTCCCTCAGGAATAAAATAATCTTGAGACTCTCAAGTGTAAATGCTGCCTCGG
GAGAACCGAACGGCGAGCTGGAGCCCATACGCAACGAGATTAGAGAGGAAGGC
AGAAGCCAGAGCACATGAATAAATGAGCATCCATTTTGTTTCAGAAATGATCGG
AAACCATTTGTGGGTTTGTAGAAGCAGGCATGCGTAGGGAAGCTACGGGATTCC
GCCGAGGAGCGCCAGAGCCTGAGGCGCCCTTTGGTTATCGCAAGCTGGCTGGCT
CACTCCGCACCAGGTGCAAAAGATGCCTGGGGATGCGGGAAGGGAAAGGCCAC
ATCTTCACGCCTTCGCGCCTGGCATTGTGAGCAACCACTGAGACTCATTATATAA
CACTCGTTTTCTTCTTGCAACCCTGCGGGCCGCGCGGTCGCGCTTTCTCTGCCCTC
CGCCGGGTGGACCTGGAGCGCTTGAGCGGTCGGCGCGCCTGGAGCAGCCAGGCG
GGCAGTGGACTAGCTGCTGGACCAGGGAGGTGTGGGAGAGCGGTGGCGGCGGG
TACATGCACGTGAAGCCATTGCGAGAACTTTATCCATAAGTATTTCAATGCCGGT
AGGGACGGCAAGAGAGGAGGGCGGGATGTGCCACACATCTTTGACCTCAGGTTT
CTAACGCCTGTTTTCTTTCTGCCCTCTGCAGACATCCCCGATTGAAAGAACCAGA
GAGGCTCTGAGAAACCTCGGGAAACTTAGATCATCAGTCACCGAAGGTCCTACA
GGGCCACAACTGCCCCCGCCACAACCCACCCCGCTTTCGTAGTTTTCATTTAGAA
AATAGAGCTTTTAAAAATGTCCTGCCTTTTAACGTAGATATATGCCTTCCCCCACT
ACCGTAAATGTCCATTTATATCATTTTTTATATATTCTTATAAAAATGTAAAAAAG
AAAAACACCGCTTCTGCCTTTTCACTGTGTTGGAGTTTTCTGGAGTGAGCACTCA
CGCCCTAAGCGCACATTCATGTGGGCATTTCTTGCGAGCCTCGCAGCCTCCGGAA
GCTGTCGACTTCATGACAAGCATTTTGTGAACTAGGGAAGCTCAGGGGGGTTACT
GGCTTCTCTTGAGTCACACTGCTAGCAAATGGCAGAACCAAAGCTCAAATAAAA
ATAAAATAATTTTCATTCATTCACTCAGTGATGCGGTTTTGGCAGTACATCAATG
GGCGTGGATAGCGGTTTGACTCACGGGGATTTcCAAGTCTCCACCCCATTGACGT
CAATGGGAGTTTGTTTTGGCACCAAAATCAACGGGACTTTCCAAAATGTCGTAAC
AACTCCGCCCCATTGACGCAAATGGGCGGTAGGCGTGTACGGTGGGAGGTCTAT
ATAAGCAGAGCTCGTTTAGTGAACCGTCAGATCGCCTGGAGACGCCATCCACGC
TGTTTTGACCTCCATAGAAGACACCGGGACCGATCCAGCCTGGGGATCCCGACAT
GGCTTCGTACCCCTGCCATCAACACGCGTCTGCGTTCGACCAGGCTGCGCGTTCT
CGCGGCCATAGCAACCGACGTACGGCGTTGCGCCCTCGCCGGCAGCAAGAAGCC
ACGGAAGTCCGCCTGGAGCAGAAAATGCCCACGCTACTGCGGGTTTATATAGAC
GGTCCTCACGGGATGGGGAAAACCACCACCACGCAACTGCTGGTGGCCCTGGGT
TCGCGCGACGATATCGTCTACGTACCCGAGCCGATGACTTACTGGCAGGTGCTGG
GGGCTTCCGAGACAATCGCGAACATCTACACCACACAACACCGCCTCGACCAGG
GTGAGATATCGGCCGGGGACGCGGCGGTGGTAATGACAAGCGCCCAGATAACAA
TGGGCATGCCTTATGCCGTGACCGACGCCGTTCTGGCTCCTCATATCGGGGGGGA
GGCTGGGAGCTCACATGCCCCGCCCCCGGCCCTCACCCTCATCTTCGACCGCCAT
CCCATCGCCGCCCTCCTGTGCTACCCGGCCGCGCGATACCTTATGGGCAGCATGA
CCCCCCAGGCCGTGCTGGCGTTCGTGGCCCTCATCCCGCCGACCTTGCCCGGCAC
AAACATCGTGTTGGGGGCCCTTCCGGAGGACAGACACATCGACCGCCTGGCCAA
ACGCCAGCGCCCCGGCGAGCGGCTTGACCTGGCTATGCTGGCCGCGATTCGCCG
CGTTTACGGGCTGCTTGCCAATACGGTGCGGTATCTGCAGGGCGGCGGGTCGTGG
CGGGAGGATTGGGGACAGCTTTCGGGGACGGCCGTGCCGCCCCAGGGTGCCGAG
CCCCAGAGCAACGCGGGCCCACGACCCCATATCGGGGACACGTTATTTACCCTGT
TTCGGGCCCCCGAGTTGCTGGCCCCCAACGGCGACCTGTACAACGTGTTTGCCTG
GGCCTTGGACGTCTTGGCCAAACGCCTCCGTCCCATGCACGTCTTTATCCTGGAT
TACGACCAATCGCCCGCCGGCTGCCGGGACGCCCTGCTGCAACTTACCTCCGGGA
TGGTCCAGACCCACGTCACCACCCCCGGCTCCATACCGACGATCTGCGACCTGGC
GCGCACGTTTGCCCGGGAGATGGGGGAGGCTAACTGA.

The vector backbone sequence (220) can be a component of the DNA immortalization construct (200) when implemented as a plasmid. The vector backbone (220) provides the structural framework for the construct, containing elements for its propagation and manipulation in bacterial systems and for transfection into mammalian cells. Typical features of a vector backbone include an origin of replication, which allows the plasmid to replicate independently in bacteria; a selectable marker, such as an antibiotic resistance gene, for selecting bacterial colonies carrying the plasmid; and multiple cloning sites, which provide convenient restriction enzyme recognition sites for inserting the DNA immortalization construct. The choice of vector backbone (220) depends on the specific requirements of the cloning, such as the desired transfection method and the target cell type. Commonly used vector backbones include those derived from pUC, pBR322, or pcDNA3.1. Variations in the vector backbone sequence are possible, as different backbones offer different features and advantages. For example, some backbones are optimized for high-copy number replication, while others are designed for stable, low-copy number maintenance. Alternative vector systems, such as viral vectors or artificial chromosomes, could be employed, but plasmids are often used because of for their ease of use, versatility, and well-established protocols for cloning and transfection. In an embodiment, the DNA immortalization construct (200), containing the hTERT gene and other elements, is inserted into a pcDNA3.1 vector backbone (220) for transfection into human primary keratinocytes. This backbone is commonly used for mammalian cell transfection and provides a robust platform for gene expression. The inclusion of a vector backbone (220) provides a practical advantage by facilitating the production, manipulation, and delivery of the DNA immortalization construct. This simplifies the process and ensures efficient transfection into target cells. The vector backbone's role in ensuring stable maintenance and replication of the construct in bacterial systems further enhances its utility for research and commercial applications.

In an embodiment, a process for immortalization begins with primary cells (302), harvested directly from human tissue. These cells are the starting material for generating immortalized cell lines. Primary cells (302) offer an advantage over established cell lines in that they retain the in vivo characteristics of their source tissue, including gene expression patterns, phenotypic markers, and functional responses. This makes them valuable for research applications where an accurate representation of in vivo cellular behavior is useful, such as studies of cellular differentiation, disease modeling, and drug discovery. The choice of primary cells (302) depends on the specific research goals and application. For instance, primary fibroblasts can be chosen for studying wound healing or skin biology, while primary hepatocytes can be selected for modeling liver function or drug metabolism. Variations in the isolation and preparation methods for primary cells (302) are possible, depending on the tissue source and the desired cell type. Commonly used techniques include enzymatic digestion, mechanical dissociation, and cell sorting. It can involve optimization of these protocols to minimize cellular damage and maintain cell viability, as healthy and actively dividing cells are more receptive to transfection and immortalization. In an embodiment, the immortalization includes isolating primary human keratinocytes from skin biopsies for generating an immortalized cell line for studying epidermal differentiation. Various enzymatic digestion and cell culture techniques can be used to isolate and expand the keratinocytes prior to transfection with the DNA immortalization construct. The use of primary cells (302) as the starting material for immortalization offers a technical advantage by ensuring that the resulting cell lines retain the in vivo characteristics of their source tissue. This makes them more reliable and representative models for research and commercial applications compared with established cell lines that may have acquired genetic and phenotypic alterations during prolonged culture.

Recipient cells (304), generated by culturing primary cells (302), can serve as the target for transfection with the DNA immortalization construct (200). Culturing primary cells (302) prior to transfection allows for expansion of the cell population, providing a sufficient number of cells for efficient gene transfer and selection. This expansion step is useful, as primary cells often have a limited lifespan and may not be readily available in large quantities. The culturing conditions, including media composition, growth factors, and temperature, are optimized for each specific cell type to promote cell growth and division while maintaining cellular health and phenotypic stability. Variations in the culture period are possible, but it can avoid excessive passaging, which can lead to replicative senescence and reduce transfection efficiency. Alternative cell sources, such as established cell lines or immortalized cell lines could be used as recipient cells, but primary cells (302) are useful as they retain the in vivo characteristics of their source tissue. In an embodiment, immortalization can include culturing primary human hepatocytes in a specialized medium supplemented with growth factors and hormones for several passages prior to transfection with the DNA immortalization construct (200). This optimized culture protocol ensures a healthy and actively dividing population of recipient cells (304) for efficient gene transfer. The use of recipient cells (304) derived from cultured primary cells offers a technical advantage by providing a larger pool of target cells for transfection, increasing the likelihood of successful gene insertion and immortalization. This expansion step maximizes the efficiency of the immortalization process and increases the yield of stable, functionally representative immortalized cell lines.

Transfected cells (306) are produced by introducing the DNA immortalization construct (200) into recipient cells (304). This gene transfer step is for integrating the hTERT gene and other elements into the cellular genome. Various transfection methods, including chemical transfection, viral transduction, and electroporation, could be employed. Homologous recombination can insert the construct at the CDKN2A gene locus. This targeted approach, using homologous arms within the construct (200) that are complementary to sequences flanking exon 2 of the CDKN2A gene, ensures precise gene insertion and minimizes the risk of off-target effects. Homologous recombination, a naturally occurring DNA repair mechanism, involves regions of sequence homology between the introduced DNA and the target genomic locus. The 5′ arm (202) and 3′ arm (218) of the DNA construct (200) provide these homologous regions, directing the integration of the construct into the CDKN2A gene. This targeted insertion disrupts the CDKN2A gene, further contributing to cell immortalization, while introducing the hTERT gene under the control of the EF1α promoter (204). In an embodiment, immortalization includes transfecting human primary fibroblasts with a linearized plasmid containing the DNA immortalization construct by electroporation. Various parameters can be leveraged such as optimizing the electroporation conditions, such as voltage, pulse duration, and DNA concentration, for maximal transfection efficiency while minimizing cell death. Comparing the efficiency of homologous recombination-mediated gene insertion with other transfection methods, such as random integration using viral vectors, shows the improved targeting specificity and reduced off-target effects of the DNA immortalization construct. The generation of transfected cells (306) carrying the DNA construct (200) at the specified locus is a step in the immortalization process, setting the stage for antibiotic selection and the establishment of stable cell lines. This targeted approach provides a technical advantage, minimizing the risk of insertional mutagenesis and enhancing the production of immortalized cells with stable genomes and predictable behavior.

The medium (308) used for culturing the transfected cells (306) plays a role in selecting for and expanding the population of immortalized cells. This medium contains the specific antibiotic (310) to which the antibiotic resistance gene (214) within the DNA immortalization construct (200) confers resistance. The inclusion of the antibiotic in the medium creates a selective pressure, allowing only transfected cells carrying the resistance gene, and thus also expressing hTERT, to survive and proliferate. Non-transfected cells, lacking the resistance gene, are unable to grow in the presence of the antibiotic and are eliminated from the culture. This selection process ensures that the resulting cell line consists exclusively of immortalized cells carrying the desired genetic modifications. The composition of the medium (308), besides the antibiotic (310), is carefully formulated to support the growth and maintenance of the specific primary cell type being immortalized. This typically includes involved nutrients, growth factors, and other components necessary for cell viability and proliferation. Variations in the medium's formulation are possible, depending on the specific requirements of the cell type, but its involved function remains providing the selective pressure necessary to isolate and expand the population of transfected cells. In an embodiment, immortalization includes using a culture medium (308) containing Zeocin (310) at a concentration of, for example, 200 μg/ml to select for immortalized human primary fibroblasts transfected with the DNA immortalization construct (200) carrying a Zeocin resistance gene (214). Other factors to control can include the basal medium, serum concentration, and any additional supplements used to support cell growth. The selective killing of non-transfected cells in the presence of Zeocin, while transfected cells survive, can confirm the effectiveness of the selection process. The use of a selective medium containing the appropriate antibiotic provides a significant technical advantage for generating pure populations of immortalized cells, a step for obtaining reliable results in research and ensuring the consistency of cell lines used in commercial applications.

The antibiotic (310) included in the medium (308) is a component of the selection process for immortalized cells. The antibiotic (310) can be chosen based on the specific antibiotic resistance gene (214) present in the DNA immortalization construct (200). This resistance gene confers the ability for transfected cells to survive and proliferate in the presence of the antibiotic (310), while non-transfected cells are eliminated. Common antibiotics used for selection include neomycin, puromycin, hygromycin, and Zeocin. The concentration of the antibiotic (310) in the medium can be optimized for each cell type to ensure effective selection without causing undue toxicity to the transfected cells. Too low a concentration may allow non-transfected cells to survive, while too high a concentration can inhibit the growth of even transfected cells. Variations in antibiotic choice and concentration are possible, depending on the cell type and the desired stringency of selection. In an embodiment, antibiotic (310) includes Zeocin (310) at a concentration of 200 μg/ml for selecting immortalized human primary keratinocytes. The selection process using the appropriate antibiotic provides a technical advantage in immortalization by enabling the efficient isolation of cells that have successfully integrated the DNA construct (200). This selection step streamlines the process of generating pure populations of immortalized cells. This produces reliable and consistent cell lines for research and commercial use.

The immortalized cell line (312), the end product of the method (300), is a tool for research, development, and commercial applications. These cells, derived from primary cells (302) and modified by the DNA immortalization construct (200), possess an extended lifespan due to the expression of the hTERT gene (208). This extended lifespan allows for long-term culture and expansion of the cells, providing a consistent and readily available source of material for experiments. The immortalized cell line (312) is characterized by its ability to divide indefinitely in vitro, bypassing the normal limitations of cellular senescence. This characteristic is confirmed by cell proliferation assays, demonstrating continuous cell growth over extended periods. The targeted insertion of the hTERT gene (208) at the CDKN2A locus minimizes disruptions to cellular function and genomic stability, resulting in immortalized cells that retain the phenotypic and functional characteristics of their primary cell counterparts. This makes them valuable tools for research applications requiring representative cell models, such as studies of cellular differentiation, disease modeling, and drug discovery. Variations in the characterization of the immortalized cell line (312) are possible, depending on the specific cell type and the intended application. For instance, one might assess the expression levels of genes and proteins, analyze cell surface markers, or evaluate functional responses to specific stimuli to confirm that the immortalized cells retain the characteristics of the primary cells (302). In an embodiment, one characterizes an immortalized human fibroblast cell line (312) by demonstrating its normal karyotype, its expression of fibroblast-specific markers, and its ability to synthesize and secrete collagen to demonstrate the long-term stability and reproducibility of the immortalized cell line (312), such as consistent growth rates and gene expression profiles over multiple passages, could also be included. The immortalized cell line (312) offers a technical advantage in research and commercial applications by providing a readily available source of cells with extended lifespans. The targeted approach to immortalization, using the DNA immortalization construct (200) and homologous recombination, minimizes phenotypic and genotypic drift, ensuring that the cell line remains representative of the original primary cells (302).

The DNA immortalization construct (200) and its components can be implemented using a variety of materials and techniques, reflecting the versatility of molecular biology and the adaptability of the invention. While embodiments described above can use a DNA-based construct, alternative approaches employing RNA or a combination of DNA and RNA could be employed. For example, the hTERT gene sequence (208) could be delivered as an RNA molecule, either directly or packaged within a suitable delivery vehicle such as a lipid nanoparticle. Similarly, the first and second nucleic acid sequences (202, 218) derived from the human CDKN2A gene can be synthesized as RNA molecules to facilitate homologous recombination with the genomic DNA. The EF1α promoter (204), while preferred for its consistent activity, can be substituted with other strong promoters, such as the CMV promoter, the CAG promoter, or promoters specific to the target cell type. The choice of promoter depends on the desired expression level and the specific requirements of the experiment. Variations in the selectable marker are also possible, using genes conferring resistance to different antibiotics or employing alternative selection strategies such as fluorescent markers or cell surface antigens. The specific selection method can be chosen based on the sensitivity and compatibility of the recipient cells (304) and the availability of appropriate detection methods. The vector backbone sequence (220) can encompass a wide range of plasmid vectors, each offering different features and advantages, or alternative vector systems such as viral vectors or artificial chromosomes can be considered. The choice of vector depends on factors such as transfection efficiency, desired expression levels, and the specific application. These variations in materials and methods demonstrate the adaptability of the DNA immortalization construct (200) and its suitability for diverse experimental conditions and cell types.

The size and dimensions of the DNA immortalization construct (200) and its constituent parts can be varied to optimize its functionality and tailor it to specific applications. While certain embodiments include a first nucleic acid sequence (202) of 4.8 kilobases and a second nucleic acid sequence (218) of 2.1 kilobases, the lengths of these homologous arms can be adjusted based on the requirements for efficient homologous recombination in the target cell type. Shorter arms may be sufficient for some applications, while longer arms could provide increased targeting specificity. The size of the human TERT gene sequence (208) is fixed, but variations in the flanking sequences, such as the inclusion of introns or regulatory elements, are possible. The length of the vector backbone sequence (220) depends on the chosen vector system, with plasmid backbones typically ranging from a few kilobases to tens of kilobases. Larger vectors can accommodate additional elements, such as reporter genes or regulatory sequences, but smaller vectors may be preferable for efficient transfection and packaging. Adjusting the size of the DNA immortalization construct (200) and its components can affect the efficiency of homologous recombination, gene expression levels, and transfection rates. A skilled artisan would consider these factors when designing a construct for a specific application, optimizing the size and dimensions to achieve the desired outcome. One can use a series of DNA constructs with varying lengths of homologous arms to assess the impact of size on targeting efficiency in human primary hepatocytes, providing optimal construct size for efficient homologous recombination in the target cell type. This flexibility in size and dimensions highlights the adaptability of the DNA immortalization construct (200) and its suitability for a broad range of cell types and experimental goals.

While certain DNA immortalization constructs (200) can be a linear sequence of connected elements, the physical shape of the construct can be varied based on the chosen delivery method and the requirements of the system. The construct can be implemented as a circular plasmid, a common format for DNA manipulation and transfection, or as a linear DNA fragment. Circular plasmids offer advantages for stable maintenance and propagation in bacterial systems, while linear DNA fragments may be preferable for certain transfection methods or for applications requiring direct genomic integration. The shape of the construct can influence its stability, transfection efficiency, and the efficiency of homologous recombination. A skilled artisan can consider these factors when selecting the appropriate construct shape for a specific application. For example, a circular plasmid containing the DNA immortalization construct for stable expression of hTERT can be used in human primary cells. Alternatively, one can use a linearized version of the construct for direct integration into the genome via homologous recombination. Accordingly, the construct has benefits afforded by various shapes in different contexts. This flexibility in construct shape highlights the adaptable nature of the invention and its applicability to a broad range of experimental systems and gene editing strategies.

DNA immortalization construct 200 can be made in various ways. The first nucleic acid sequence (202) and the second nucleic acid sequence (218), derived from the human CDKN2A gene, can be obtained by PCR amplification from genomic DNA, followed by cloning into a suitable vector. Alternatively, these sequences can be chemically synthesized, offering precise control over their length and composition. The EF1α promoter sequence (204), the LoxP sequences (206, 210), the hTERT gene sequence (208), the IRES sequence (212), and the antibiotic resistance gene sequence (214) can similarly be obtained by PCR or chemical synthesis. Commercial gene synthesis services provide a convenient option for obtaining these sequences with high accuracy and speed. The assembly of the DNA immortalization construct (200) can be achieved using standard molecular cloning techniques, such as restriction enzyme digestion and ligation, Gibson assembly, or Golden Gate cloning. The assembled construct is typically cloned into a plasmid vector backbone (220) for propagation and manipulation in bacterial systems. The choice of vector depends on the specific experimental needs, with certain vectors offering advantages for homologous recombination or high-level gene expression. The linearized construct, or the plasmid containing the construct, can be purified using standard techniques such as gel electrophoresis or column chromatography before transfection into recipient cells. The specifics of the transfection method, while dependent on the recipient cell type and the chosen vector, can be optimized for efficient gene transfer and integration. Common methods include electroporation, lipofection, or viral transduction. Following transfection, selection for cells carrying the integrated construct is achieved by culturing the cells in a medium containing the appropriate antibiotic, as dictated by the antibiotic resistance gene sequence (214). The concentration of the antibiotic is titrated to ensure effective selection without causing undue toxicity to the cells. Confirmation of successful integration can be achieved by PCR analysis using primers specific for the recombinant allele, or by Southern blotting. Characterization of the resulting immortalized cell line (312) is involved for verifying that the cells retain the desired phenotypic and functional characteristics. This characterization can include analysis of gene expression, cell surface markers, karyotype, and functional assays relevant to the specific cell type and intended application. The disclosed sequences and methods provide a foundation to generate custom DNA immortalization constructs targeting various genomic loci and incorporating diverse regulatory elements. This versatility enhances the utility of the invention for a wide range of research endeavors.

In an embodiment, a method (400) for producing a DNA immortalization construct (200) comprises: providing a first nucleic acid sequence (202) comprising about 4.8 kilobases and derived from the 5′ end of a human CDKN2A gene exon 2; providing an EF1α promoter sequence (204); providing a first LoxP sequence (206); providing a human TERT gene sequence (208); providing a second LoxP sequence (210); providing an internal ribosomal entry site (IRES) sequence (212); providing an antibiotic resistance gene sequence (214); providing an SV40 poly-A signal sequence (216); providing a second nucleic acid sequence (218) comprising about 2.1 kilobases and derived from the 3′ end of the human CDKN2A gene exon 2; and sequentially connecting the first nucleic acid sequence (202), the EF1α promoter sequence (204), the first LoxP sequence (206), the human TERT gene sequence (208), the second LoxP sequence (210), the IRES sequence (212), the antibiotic resistance gene sequence (214), the SV40 poly-A signal sequence (216), and the second nucleic acid sequence (218) to produce the DNA immortalization construct (200). In an embodiment, providing the first nucleic acid sequence (202) comprises chemically synthesizing the sequence. In an embodiment, providing the second nucleic acid sequence (218) comprises chemically synthesizing the sequence. In an embodiment, providing the EF1α promoter sequence (204) comprises obtaining the sequence from a preexisting vector. In an embodiment, providing the human TERT gene sequence (208) comprises synthesizing the sequence from an RNA template. In an embodiment, the antibiotic resistance gene sequence (214) confers resistance to Zeocin. In an embodiment, the method further comprises inserting the sequentially connected sequences into a vector backbone (220). In an embodiment, the vector backbone (220) is a plasmid. In an embodiment, the method further comprises linearizing the plasmid comprising the DNA immortalization construct (200). In an embodiment, linearizing comprises digesting the plasmid with a restriction endonuclease.

In an embodiment, producing the DNA immortalization construct (200) begins by providing a first nucleic acid sequence (202) comprising about 4.8 kilobases and derived from the 5′ end of a human CDKN2A gene exon 2. This sequence, referred to as the 5′ arm, serves as a homologous region for targeted integration via homologous recombination. It provides a region of DNA identity with the target CDKN2A locus, enabling precise insertion of the construct. The EF1α promoter sequence (204), a strong and constitutively active promoter, drives the expression of the downstream hTERT gene, ensuring robust and consistent transcription. The inclusion of a first LoxP sequence (206) upstream of the hTERT gene creates a site for Cre-mediated recombination. This sequence, in conjunction with a second LoxP sequence (210) placed downstream of the hTERT gene, enables conditional removal of the hTERT gene, providing flexibility for researchers to control its expression. The human TERT gene sequence (208), encoding the catalytic subunit of telomerase, is the core element responsible for extending the replicative lifespan of primary cells. This gene, placed under the control of the EF1α promoter (204), allows cells to overcome telomere shortening and bypass senescence. The second LoxP sequence (210), identical to the first, provides the necessary second recognition site for Cre recombinase, enabling conditional hTERT gene removal. An internal ribosomal entry site (IRES) sequence (212) facilitates the co-expression of the hTERT gene and a selectable marker from a single mRNA transcript. This simplifies construct design and ensures that all cells expressing hTERT also express the selectable marker, allowing for efficient selection of transfected cells. The inclusion of an antibiotic resistance gene sequence (214) confers resistance to a specific antibiotic, enabling the selection and isolation of cells that have successfully integrated the DNA construct. An SV40 poly-A signal sequence (216), located downstream of the antibiotic resistance gene sequence (214), enhances mRNA stability and translation efficiency, boosting expression levels of both hTERT and the antibiotic resistance protein. Finally, a second nucleic acid sequence (218) comprising about 2.1 kilobases and derived from the 3′ end of the human CDKN2A gene exon 2 serves as a second homologous arm, further enhancing the efficiency of homologous recombination at the target locus. These elements are sequentially connected to produce the complete DNA immortalization construct (200), a powerful tool for generating stable and functionally representative immortalized cell lines.

The method (400) for producing the DNA immortalization construct (200) can include incorporating various techniques and materials employed in molecular biology. Chemically synthesizing the first nucleic acid sequence (202) and the second nucleic acid sequence (218) offers a precise and efficient approach for generating the homologous arms of the construct. Chemical DNA synthesis utilizes phosphoramidite chemistry to assemble oligonucleotides, short DNA sequences, into longer fragments. This method allows for the incorporation of specific modifications, such as restriction enzyme recognition sites or mutations, into the homologous arms, enhancing their functionality or facilitating downstream cloning steps. Obtaining the EF1α promoter sequence (204) from a preexisting vector provides a convenient and reliable source for this element. Numerous commercially available vectors contain the EF1α promoter, simplifying its isolation and incorporation into the DNA immortalization construct. Synthesizing the human TERT gene sequence (208) from an RNA template utilizes reverse transcription, a technique that converts RNA into complementary DNA. This approach can be particularly useful when the hTERT gene sequence contains introns or other modifications that may be challenging to synthesize directly. Employing an antibiotic resistance gene (214) conferring resistance to Zeocin offers a versatile and efficient selection strategy for isolating transfected cells. Zeocin, a broad-spectrum antibiotic, inhibits protein synthesis in both prokaryotic and eukaryotic cells. The Zeocin resistance gene encodes a protein that inactivates Zeocin, enabling transfected cells to survive in selective media containing the antibiotic. Inserting the sequentially connected sequences into a vector backbone (220), such as a plasmid, simplifies the construct's propagation and manipulation in bacterial systems and facilitates its transfection into mammalian cells. Plasmids are circular, double-stranded DNA molecules that replicate independently in bacteria, providing a convenient system for amplifying and storing the DNA construct. Using a plasmid vector also enables the introduction of additional elements, such as reporter genes or regulatory sequences, into the construct. Linearizing the plasmid containing the DNA immortalization construct (200) prepares it for transfection into mammalian cells. Linearization, typically achieved by digesting the plasmid with a restriction endonuclease, ensures efficient integration into the genome, particularly for homologous recombination. The choice of restriction enzyme depends on the available recognition sites within the plasmid and the desired integration strategy.

The initial step in producing the DNA immortalization construct (200) involves providing a first nucleic acid sequence (202) comprising about 4.8 kilobases and derived from the 5′ end of a human CDKN2A gene exon 2. This sequence, designated as the 5′ arm, plays a role in targeted integration of the construct via homologous recombination. The 4.8 kilobase sequence can be obtained through various methods employed in molecular biology. One approach involves polymerase chain reaction (PCR) amplification from a genomic DNA template using primers specific for the desired region flanking the 5′ end of exon 2. The amplified PCR product is then purified and cloned into a suitable vector for propagation and sequence verification. Alternatively, the 4.8 kilobase sequence can be chemically synthesized using commercially available oligonucleotide synthesis services. Chemical synthesis allows for precise control over the sequence and can incorporate specific modifications, such as restriction enzyme recognition sites, if desired. The choice of method depends on factors such as the availability of a suitable genomic DNA template, the desired accuracy, and the cost and time constraints of the experiment. Regardless of the chosen method, the resulting 4.8 kilobase sequence can accurately represent the target region of the human CDKN2A gene to ensure efficient homologous recombination during the immortalization process.

Providing an EF1α promoter sequence (204) is a useful step in constructing the DNA immortalization construct (200), as this promoter drives high-level expression of the hTERT gene (208). The EF1α promoter, a strong and constitutively active promoter derived from the human elongation factor 1 alpha gene, ensures robust and consistent transcription of the hTERT gene, leading to high levels of telomerase expression and efficient cell immortalization. The EF1α promoter sequence (204) can be obtained through various methods employed in molecular biology. A straightforward approach is to isolate the sequence from a preexisting vector containing the EF1α promoter. Numerous commercially available plasmids contain this promoter, making it readily accessible for cloning purposes. Alternatively, the EF1α promoter sequence can be chemically synthesized using oligonucleotide synthesis services, allowing for precise control over the sequence and the incorporation of specific modifications, such as restriction enzyme recognition sites. The choice of method depends on the specific experimental needs and the availability of resources. Once obtained, the EF1α promoter sequence (204) is typically amplified by PCR and cloned into a suitable vector, ready for assembly into the complete DNA immortalization construct.

Providing a first LoxP sequence (206) is a step in enabling conditional regulation of hTERT expression within the DNA immortalization construct (200). LoxP sequences, 34-base pair DNA sequences recognized by the Cre recombinase enzyme, allow for the precise excision of DNA flanked by these sites. The first LoxP sequence (206) is positioned upstream of the human TERT gene sequence (208) and, together with a second LoxP sequence (210) downstream of the hTERT gene, creates a removable hTERT expression cassette. The first LoxP sequence (206) can be incorporated into the construct through various techniques. One approach involves including the sequence within the primers used for PCR amplification of the EF1α promoter (204) or the hTERT gene (208). This ensures that the LoxP sequence is incorporated during the amplification process. Alternatively, the LoxP sequence can be added through restriction enzyme digestion and ligation. Commercially available plasmids containing LoxP sites can serve as a convenient source for obtaining the sequence. The chosen method depends on the specific experimental design and the available cloning tools. The precise sequence and orientation of the LoxP site are useful for efficient Cre-mediated recombination, ensuring controlled removal of the hTERT expression cassette when desired.

Providing a human TERT gene sequence (208) involves precise and reliable methods to ensure the fidelity and functionality of the gene. The hTERT gene, encoding the catalytic subunit of telomerase, is responsible for extending the replicative lifespan of immortalized cells. The hTERT gene sequence (208) can be obtained through several approaches. One common method involves reverse transcription PCR (RT-PCR) using an RNA template isolated from cells expressing high levels of hTERT. The resulting cDNA is then amplified using PCR with primers specific for the hTERT coding sequence. This method allows for the generation of a full-length hTERT cDNA, including any naturally occurring variations or modifications present in the RNA template. Alternatively, the hTERT gene sequence (208) can be chemically synthesized using commercially available gene synthesis services. This method offers precise control over the sequence and can incorporate specific modifications, such as codon optimization for enhanced expression in specific cell types, or the addition of tags for downstream protein detection or purification. Regardless of the chosen method, the resulting hTERT gene sequence (208) is typically cloned into a suitable vector for sequence verification and subsequent assembly into the DNA immortalization construct (200). Ensuring the accuracy and integrity of the hTERT sequence is useful for the functionality of the construct and the successful immortalization of primary cells.

Providing a second LoxP sequence (210) is involved for completing the hTERT expression cassette within the DNA immortalization construct (200), enabling its conditional removal by Cre recombinase. This second LoxP sequence (210), identical to the first LoxP sequence (206), is positioned downstream of the human TERT gene sequence (208). The two LoxP sites, flanking the hTERT gene, create a removable cassette, allowing for controlled excision of the gene when desired. The second LoxP sequence (210) can be incorporated into the construct using similar methods as those described for the first LoxP sequence. It can be included in the PCR primers used to amplify the hTERT gene (208) or the downstream sequences, ensuring its presence in the final construct. Alternatively, the second LoxP sequence (210) can be added through restriction enzyme digestion and ligation, using commercially available plasmids containing LoxP sites as a source. The precise placement and orientation of the second LoxP site are involved, as they must be compatible with the first LoxP site (206) to allow for efficient Cre-mediated recombination and excision of the hTERT gene (208).

Providing an internal ribosomal entry site (IRES) sequence (212) is a step in facilitating the co-expression of multiple genes from a single mRNA transcript within the DNA immortalization construct (200). IRES sequences allow for cap-independent translation initiation, enabling the production of multiple proteins from a single mRNA molecule. In the context of the DNA immortalization construct, the IRES sequence (212) is positioned downstream of the second LoxP sequence (210) and upstream of the antibiotic resistance gene sequence (214), allowing for the simultaneous translation of both the hTERT gene (208) and the antibiotic resistance gene from a single transcript driven by the EF1α promoter (204). The IRES sequence (212) can be obtained from various sources, including commercially available plasmids or by PCR amplification from a template containing a known IRES element. Different IRES sequences exhibit varying degrees of translational efficiency, and the choice of IRES depends on the desired expression levels of the downstream genes and the compatibility with the specific cell type being immortalized. Once obtained, the IRES sequence (212) is typically amplified by PCR and cloned into a suitable vector for subsequent assembly into the DNA immortalization construct (200). The IRES sequence's position and orientation within the construct are useful for its proper function, ensuring efficient translation of both the hTERT and antibiotic resistance genes.

Providing an antibiotic resistance gene sequence (214) is involved for enabling selection of cells that have successfully integrated the DNA immortalization construct (200). This gene confers resistance to a specific antibiotic, allowing transfected cells to survive in a selective medium containing that antibiotic, while non-transfected cells are eliminated. The choice of antibiotic resistance gene (214) depends on the specific cell type being immortalized and the availability of appropriate selection methods. Commonly used antibiotic resistance genes include those conferring resistance to neomycin, puromycin, hygromycin, or Zeocin. These genes can be obtained from various sources, including commercially available plasmids or by PCR amplification from a template containing a known resistance gene. The selected antibiotic resistance gene sequence (214) is typically amplified by PCR and cloned into a suitable vector, ready for assembly into the complete DNA immortalization construct. It is involved to ensure that the resistance gene is compatible with the chosen cell type and that the corresponding antibiotic is effective in selecting for transfected cells.

Providing an SV40 poly-A signal sequence (216) is a useful step in enhancing the expression levels of both the hTERT gene (208) and the antibiotic resistance gene (214) within the DNA immortalization construct (200). The SV40 poly-A signal, derived from the simian virus 40, is a commonly used and highly efficient polyadenylation signal. It directs the addition of a poly-A tail, a string of adenine nucleotides, to the 3′ end of mRNA transcripts, which is involved for mRNA stability and translation efficiency. The SV40 poly-A signal sequence (216) can be obtained from various sources, including commercially available plasmids or by PCR amplification from a template containing the SV40 poly-A signal. The sequence is typically cloned into a suitable vector for subsequent assembly into the DNA immortalization construct (200). Its placement downstream of the antibiotic resistance gene (214) ensures that both the hTERT and antibiotic resistance transcripts are properly polyadenylated, enhancing their stability and translation. The inclusion of the SV40 poly-A signal (216) contributes to the overall efficiency of the construct by increasing the levels of hTERT and the antibiotic resistance protein in transfected cells, leading to more robust immortalization and selection.

Providing a second nucleic acid sequence (218) comprising about 2.1 kilobases and derived from the 3′ end of the human CDKN2A gene exon 2 is involved for completing the DNA immortalization construct (200). This sequence, referred to as the 3′ arm, serves as a second homologous region for targeted integration, similar to the 5′ arm (202). The inclusion of two homologous arms flanking the hTERT expression cassette enhances the efficiency and precision of homologous recombination at the CDKN2A locus. The 2.1 kilobase 3′ arm (218) can be obtained using the same techniques described for providing the 4.8 kilobase 5′ arm (202). It can be amplified by PCR from a genomic DNA template using primers specific for the desired region flanking the 3′ end of exon 2 or synthesized chemically using oligonucleotide synthesis services. The resulting sequence is then cloned into a suitable vector for propagation and sequence verification. The length of the 3′ arm can be adjusted based on the specific requirements of the target cell type and the efficiency of homologous recombination. However, a 2.1 kilobase sequence provides a sufficient length for effective homologous recombination in most cases. The 3′ arm sequence (218) is involved for ensuring precise integration of the construct, minimizing the risk of random insertion events that can disrupt gene function or lead to genomic instability.

Sequentially connecting the individual components—the first nucleic acid sequence (202), the EF1α promoter sequence (204), the first LoxP sequence (206), the human TERT gene sequence (208), the second LoxP sequence (210), the IRES sequence (212), the antibiotic resistance gene sequence (214), the SV40 poly-A signal sequence (216), and the second nucleic acid sequence (218)—to produce the complete DNA immortalization construct (200) involves precision and a systematic approach to ensure the correct arrangement and functionality of the final product. This assembly process typically involves standard molecular cloning techniques. Each component is first cloned into a separate vector, allowing for sequence verification and manipulation. The components are then sequentially excised from their respective vectors using restriction enzymes and ligated together in the desired order. Alternative cloning methods, such as Gibson assembly or Golden Gate cloning, can also be employed, offering advantages in speed and efficiency. The final assembled construct is typically cloned into a suitable vector backbone, such as a plasmid, for propagation and manipulation in bacterial systems and for subsequent transfection into mammalian cells. The precise order of the components within the construct is involved for its functionality, as it dictates the expression and regulation of the hTERT gene and the selectable marker. Attention to detail during the assembly process can ensure the production of a functional DNA immortalization construct capable of efficiently immortalizing primary cells.

Verification of each component's sequence and orientation within the construct is involved for ensuring its functionality. This verification can be achieved through standard techniques such as Sanger sequencing or restriction enzyme digestion analysis. Following assembly, the construct is typically cloned into a suitable vector backbone (220) for propagation and amplification in bacterial cells. This involves transforming competent bacteria with the construct and selecting for colonies carrying the plasmid using the appropriate antibiotic resistance marker. The amplified plasmid DNA is then isolated and purified using commercially available kits or standard protocols. Before transfection into recipient cells, the DNA construct (200) may require linearization to enhance integration into the genome. Linearization can be achieved by digesting the plasmid with a restriction enzyme or by PCR amplification of the construct. The choice of method depends on the specific requirements of the transfection protocol and the desired integration strategy. The transfection method can be optimized for efficient gene transfer into the target cell type. Various methods, including chemical transfection, electroporation, and viral transduction, can be employed, with the choice depending on factors such as cell type, transfection efficiency, and the desired expression levels. Following transfection, selection for cells carrying the integrated construct is achieved by culturing the cells in a selective medium containing the appropriate antibiotic. The concentration of the antibiotic should be optimized to ensure effective selection without causing undue toxicity to the transfected cells. Confirmation of successful integration and expression of the hTERT gene can be achieved by various methods, including PCR analysis, Southern blotting, or immunoblotting. These steps can be practiced for implementation of the method (400) and ensure the production of a functional and reliable DNA immortalization construct (200). The disclosed methods and techniques provide a robust framework for generating a wide range of immortalized cell lines, enabling researchers to tailor the construct to their specific needs and advance research in diverse fields.

DNA immortalization construct 200 has numerous advantageous and unexpected benefits and uses. In an embodiment, a method (300) for immortalizing primary cells comprises: providing a DNA immortalization construct (200) comprising a first nucleic acid sequence (202) derived from the 5′ end of a human CDKN2A gene exon 2; an EF1α promoter sequence (204) connected to the first sequence (202); a human TERT gene sequence (208) connected to the EF1α promoter sequence (204); an antibiotic resistance gene sequence (214); and a second nucleic acid sequence (218) derived from the 3′ end of the human CDKN2A gene exon 2; isolating primary cells (302) from human tissue; culturing the primary cells (302) to produce recipient cells (304); transfecting the recipient cells (304) with the DNA immortalization construct (200) using homologous recombination to produce transfected cells (306); culturing the transfected cells (306) in a medium (308) comprising an antibiotic (310) to select for transfected cells exhibiting antibiotic resistance; and isolating the transfected cells (306) exhibiting resistance to the antibiotic (310) to produce an immortalized cell line (312). In an embodiment, the method further comprises linearizing the DNA immortalization construct (200) prior to transfecting the recipient cells (304). In an embodiment, linearizing comprises digesting the DNA immortalization construct (200) with a restriction enzyme. In an embodiment, the restriction enzyme is PvuI, e.g., from the bacteria Proteus vulgaris. In an embodiment, transfecting comprises electroporation. In an embodiment, the method further comprises: expanding the immortalized cell line (312); and cryopreserving the immortalized cell line (312). In an embodiment, the primary cells (302) are CD8+ T-cells. In an embodiment, the immortalized cell line (312) comprises a single copy of the human TERT gene sequence (208) inserted into the CDKN2A gene. In an embodiment, the immortalized cell line (312) exhibits a decreased expression of the CDKN2A gene. In an embodiment, the immortalized cell line (312) exhibits a normal karyotype and expresses cell surface markers consistent with the primary cells (302).

The method for performing primary cell immortalization addresses limitations of conventional techniques and offers a more precise and controlled approach to generating stable cell lines. The method can begin with providing a DNA immortalization construct (200) designed to introduce specific genetic modifications into primary cells. This construct comprises several elements, including a first nucleic acid sequence (202) and a second nucleic acid sequence (218) derived from the 5′ and 3′ ends of the human CDKN2A gene exon 2, respectively. These sequences, referred to as homologous arms, facilitate targeted integration of the construct at the CDKN2A locus via homologous recombination. The construct also includes an EF1α promoter sequence (204), a strong and constitutively active promoter, that drives high-level expression of the downstream human TERT gene sequence (208), an element for extending cellular lifespan. An antibiotic resistance gene (214) within the construct enables selection for cells that have successfully integrated the DNA. The method then involves isolating primary cells (302) from human tissue, the starting material for generating the immortalized cell line. These primary cells are cultured to produce recipient cells (304), expanding the cell population and ensuring sufficient numbers for subsequent transfection. The recipient cells are then transfected with the DNA immortalization construct (200) using homologous recombination, a precise gene-editing technique that relies on the homology between the construct's arms and the target CDKN2A gene locus. This targeted approach minimizes the risk of insertional mutagenesis and ensures the construct's integration at the desired location. The transfected cells (306) are then cultured in a medium (308) containing a specific antibiotic (310) corresponding to the antibiotic resistance gene (214) within the construct. This selection step allows only transfected cells, carrying the resistance gene and thus expressing hTERT, to survive and proliferate. Finally, the transfected cells (306) exhibiting resistance to the antibiotic (310) are isolated to produce the immortalized cell line (312), a population of cells capable of continuous cell division in vitro.

The immortalization method (300) can include various techniques employed in molecular biology and cell culture. Linearizing the DNA immortalization construct (200) prior to transfection can enhance its integration into the genome. Linearization, typically achieved by digesting the construct with a restriction enzyme, converts the circular plasmid into a linear DNA molecule, which is often more readily taken up by cells and integrated into the genomic DNA. The choice of restriction enzyme depends on the specific plasmid vector (220) used and the presence of appropriate recognition sites. Digesting the DNA immortalization construct (200) with the restriction enzyme PvuI provides a specific and efficient method for linearization. PvuI recognizes a six-base pair sequence and cuts the DNA in a blunt-ended fashion, generating a linearized fragment suitable for transfection. Transfecting the recipient cells (304) via electroporation offers a versatile and efficient method for introducing the DNA construct (200) into the cells. Electroporation involves applying a brief electrical pulse to the cells, creating temporary pores in the cell membrane that allow for the entry of DNA. The parameters of the electrical pulse, such as voltage and duration, are optimized for each cell type to maximize transfection efficiency while minimizing cell death. Expanding the immortalized cell line (312) after selection involves culturing the cells under optimal conditions to increase their number and generate sufficient material for downstream applications. This expansion step ensures a readily available supply of cells for research or commercial use. Cryopreserving the immortalized cell line (312) involves storing the cells at ultra-low temperatures, typically in liquid nitrogen, to maintain their viability and genetic stability for long-term storage. This allows researchers to create a cell bank and access the cells as needed without having to repeat the immortalization process. Using CD8+ T-cells as the primary cells (302) offers a specific application of the immortalization method, enabling the generation of a valuable tool for studying T-cell biology and developing immunotherapies. CD8+ T-cells play a role in cell-mediated immunity, recognizing and killing infected or cancerous cells. An immortalized CD8+ T-cell line can be used to study T-cell activation, differentiation, and function in vitro, facilitating the development of new immunotherapies. Generating an immortalized cell line (312) that comprises a single copy of the human TERT gene sequence (208) inserted into the CDKN2A gene ensures controlled hTERT expression and minimizes potential off-target effects. This targeted insertion approach, achieved through homologous recombination, replaces a portion of the CDKN2A gene with the hTERT expression cassette, leading to both hTERT overexpression and disruption of the CDKN2A gene. The CDKN2A gene encodes for proteins that regulate cell cycle progression and senescence, and its disruption further contributes to cell immortalization. The immortalized cell line (312) will exhibit decreased expression of the CDKN2A gene as a result of the targeted gene disruption. This decreased expression can be confirmed by various techniques, such as RT-PCR or Western blotting, demonstrating the loss of CDKN2A function. An immortalized cell line (312) that exhibits a normal karyotype and expresses cell surface markers consistent with the primary cells (302) from which it was derived indicates that the immortalization process has not significantly altered the cells' genetic makeup or phenotypic characteristics. This ensures that the cell line remains representative of the original primary cells and is suitable for use in research applications requiring accurate cellular models.

Isolating primary cells (302) from human tissue provides the foundation for generating a cell line that retains the in vivo characteristics of its source. Primary cells, unlike established cell lines, are directly harvested from living tissue, offering a more accurate representation of the cellular phenotype and function found in the human body. The isolation process involves separating the desired cell type from the surrounding tissue and creating a pure population of viable cells suitable for culture and subsequent modification. The specific techniques used for isolating primary cells (302) depend on the tissue source and the desired cell type. Commonly employed methods include enzymatic digestion, mechanical dissociation, and cell sorting. Enzymatic digestion involves using enzymes, such as collagenase or trypsin, to break down the extracellular matrix and release individual cells from the tissue. Mechanical dissociation uses physical methods, such as gentle pipetting or scraping, to separate cells from the tissue. Cell sorting techniques, such as fluorescence-activated cell sorting (FACS) or magnetic-activated cell sorting (MACS), can isolate specific cell populations based on the expression of cell surface markers or other characteristics. The choice of isolation method depends on the tissue's properties, the desired cell type, and the availability of specific reagents or equipment. The isolated cells are typically cultured in a specialized medium formulated to support the growth and viability of the specific cell type. Careful optimization of the isolation and culture protocols is involved to minimize cellular damage, maintain cell viability, and preserve the phenotypic characteristics of the primary cells (302). In an embodiment, the method includes isolating primary human hepatocytes from liver tissue using a combination of enzymatic digestion and density gradient centrifugation. The method can be optimized by selecting specific enzymes for digestion, the centrifugation parameters, and the composition of the culture medium used to maintain the isolated hepatocytes. The use of primary cells (302) as the starting material for immortalization ensures that the resulting cell line reflects the in vivo characteristics of the source tissue, providing a valuable tool for research and development.

Culturing the primary cells (302) to produce recipient cells (304) provides expansion of the primary cell population and generating a sufficient number of cells for subsequent transfection and selection. Primary cells, harvested directly from tissue, often exist in limited numbers and may not be immediately suitable for large-scale transfection. Culturing these cells under controlled conditions allows for their expansion and provides a readily available pool of recipient cells (304) ready for genetic modification. The culturing process involves maintaining the primary cells (302) in a specialized medium formulated to support their growth and proliferation. This medium typically contains involved nutrients, growth factors, and other components necessary for cell viability and division. The culture conditions, including temperature, humidity, and gas exchange, are carefully controlled to mimic the in vivo environment as closely as possible. Variations in the culture medium and conditions are possible, depending on the specific requirements of the primary cell type. For instance, some cells may require additional growth factors or specific serum concentrations for optimal growth. The duration of the culture period depends on the growth rate of the primary cells (302) and the desired number of recipient cells (304) required for transfection. However, it is important to avoid excessive passaging, as prolonged culture can lead to phenotypic drift and reduced transfection efficiency. The process can involve culturing primary human fibroblasts in a medium containing Dulbecco's Modified Eagle Medium (DMEM) supplemented with 10% fetal bovine serum and specific growth factors for several passages to generate a sufficient number of recipient cells for transfection. The method can control for the optimal culture conditions, such as temperature and humidity, used to maintain cell viability and promote cell proliferation. The expansion of the primary cell population through culturing ensures a sufficient number of recipient cells (304) for efficient transfection and subsequent selection, maximizing the yield of immortalized cells and contributing to the overall success of the immortalization process.

Transfecting the recipient cells (304) with the DNA immortalization construct (200) introduces the genetic modifications necessary for extending cellular lifespan and establishing a stable cell line. This process involves introducing the DNA construct into the recipient cells and facilitating its integration into the cellular genome. Homologous recombination can be the mechanism for transfection, ensuring precise targeting of the construct to the CDKN2A gene locus. Homologous recombination, a naturally occurring DNA repair process, relies on the exchange of genetic material between two DNA molecules with similar sequences. The DNA immortalization construct (200) is designed to exploit this mechanism by incorporating two homologous arms, the first nucleic acid sequence (202) and the second nucleic acid sequence (218), which share sequence identity with regions flanking exon 2 of the human CDKN2A gene. These homologous arms guide the integration of the construct into the target locus, resulting in the replacement of exon 2 with the hTERT expression cassette and the selectable marker. This targeted approach minimizes the risk of random integration, a common concern with conventional methods like viral transduction, where the integration site is unpredictable and can disrupt gene function or lead to genomic instability. Various methods can be employed to introduce the DNA construct (200) into recipient cells (304), including chemical transfection, electroporation, and viral transduction. The choice of method depends on the specific cell type and the characteristics of the DNA construct, with certain methods being more efficient or suitable for specific applications. The method can include transfecting human primary fibroblasts with a linearized plasmid containing the DNA immortalization construct using electroporation. The method can include optimization of electroporation parameters, such as voltage, pulse duration, and DNA concentration, to maximize transfection efficiency and cell viability to provide homologous recombination-mediated gene insertion in an absence of random integration using viral vectors for improved targeting specificity and reduced off-target effects of the construct. The successful integration of the DNA construct (200) into the recipient cells (304) results in the generation of transfected cells (306) carrying the desired genetic modifications, including the hTERT gene for extended lifespan and the antibiotic resistance gene for selection.

Culturing the transfected cells (306) in a medium (308) containing an antibiotic (310) provides selection and expansion of cells that have successfully integrated the DNA immortalization construct (200). The antibiotic (310) is chosen based on the specific antibiotic resistance gene (214) present in the construct. This gene confers resistance to the antibiotic, allowing transfected cells carrying the gene to survive and proliferate in the selective medium, while non-transfected cells, lacking the resistance gene, are eliminated. This selective pressure ensures that only cells carrying the desired genetic modifications, including the hTERT gene for extended lifespan, are propagated. The concentration of the antibiotic (310) in the medium (308) is carefully optimized for each cell type to ensure efficient selection without causing excessive toxicity to the transfected cells. Too low a concentration may allow non-transfected cells to survive, while too high a concentration can inhibit the growth of even transfected cells. The optimal concentration typically falls within a narrow range, determined empirically through cell viability assays. The culture medium (308), in addition to the antibiotic (310), contains involved nutrients, growth factors, and other components necessary to support the growth and proliferation of the specific primary cell type being immortalized. The culture conditions, such as temperature, humidity, and gas exchange, are also carefully controlled to maintain cellular health and viability. Variations in the culture medium (308) and conditions are possible, depending on the specific requirements of the cell type being immortalized. The method can include culturing human primary fibroblasts transfected with a construct containing a Zeocin resistance gene in a medium containing DMEM supplemented with 10% fetal bovine serum, specific growth factors, and Zeocin at a concentration of 200 μg/ml, wherein growth curves of transfected and non-transfected cells in the presence and absence of Zeocin show the selective pressure exerted by the antibiotic. This selection step ensures that only cells carrying the DNA immortalization construct (200), and therefore expressing hTERT, are expanded, resulting in a pure population of immortalized cells. This selectivity is involved for generating a stable and functionally representative cell line suitable for research and commercial applications.

Isolating the transfected cells (306) exhibiting resistance to the antibiotic (310) provides establishment of a stable, continuously dividing cell line (312). After culturing the transfected cells in the selective medium, only those cells that have successfully integrated the DNA immortalization construct (200) and are expressing the antibiotic resistance gene (214) will survive. These cells, now resistant to the antibiotic (310), can be isolated and expanded to generate the immortalized cell line (312). This isolation can be achieved through various methods, depending on the scale of the experiment and the desired purity of the cell population. For small-scale experiments, manual isolation using a microscope and sterile techniques might be sufficient. For larger-scale applications, techniques like limiting dilution cloning, where individual cells are seeded into separate wells, can be used to isolate single-cell clones and ensure a genetically homogeneous cell population. The isolated cells are then expanded in culture under optimal conditions to generate the immortalized cell line (312), a population of cells capable of continuous division in vitro. The method can include isolating Zeocin-resistant human primary fibroblasts after transfection with the DNA immortalization construct (200) by limiting dilution cloning, wherein the cell seeding density and single-cell clones can be confirmed with microscopic analysis. Data for such can show doubling time and saturation density that confirms the establishment of a stable, immortalized cell line (312). This isolation step provides a technical advantage by ensuring the purity and homogeneity of the immortalized cell line (312), involved for generating reliable and reproducible results in research and for producing consistent cell lines for commercial applications.

In addition to various foregoing procedural aspects, performing primary cell immortalization can include sundry ancillary procedures commonly employed in cell culture and molecular biology. Before transfecting recipient cells (304), the DNA immortalization construct (200) can be subject to preparation. If delivered as a plasmid, the construct can be linearized to facilitate integration into the host genome. This can be achieved by digesting the plasmid with a suitable restriction enzyme, chosen based on its recognition sequence within the plasmid backbone. The linearized construct is then purified using techniques such as gel electrophoresis or column chromatography to remove any residual enzymes or buffer components. The specific parameters of the transfection protocol, while dependent on the chosen method and the recipient cell type, involve optimization for efficient gene transfer and cell viability. For instance, if using electroporation, the voltage, pulse duration, and DNA concentration can be titrated to maximize transfection efficiency while minimizing cell death. Following transfection, a recovery period may be necessary to allow cells to recover from the stress of the procedure and begin expressing the introduced genes. This typically involves culturing the cells in a non-selective medium for a short period before applying antibiotic selection. During antibiotic selection, monitoring cell growth and viability is involved to ensure optimal selection pressure. Too rapid cell death can indicate excessive antibiotic concentration, while slow or incomplete selection can suggest use of a higher antibiotic concentration or a longer selection period. After establishing the immortalized cell line (312), additional characterization steps can confirm its suitability for specific applications. Analyzing the karyotype of the cells ensures that the immortalization process has not introduced significant chromosomal abnormalities. Assessing cell surface marker expression, using techniques like flow cytometry or immunostaining, verifies that the cells retain the phenotypic characteristics of their primary cell counterparts. Functional assays, tailored to the specific cell type and research goals, can confirm the preservation of cellular functions. These additional steps can be useful for ensuring the quality and reliability of the immortalized cell line (312) and for maximizing its utility for research, development, and commercial applications.

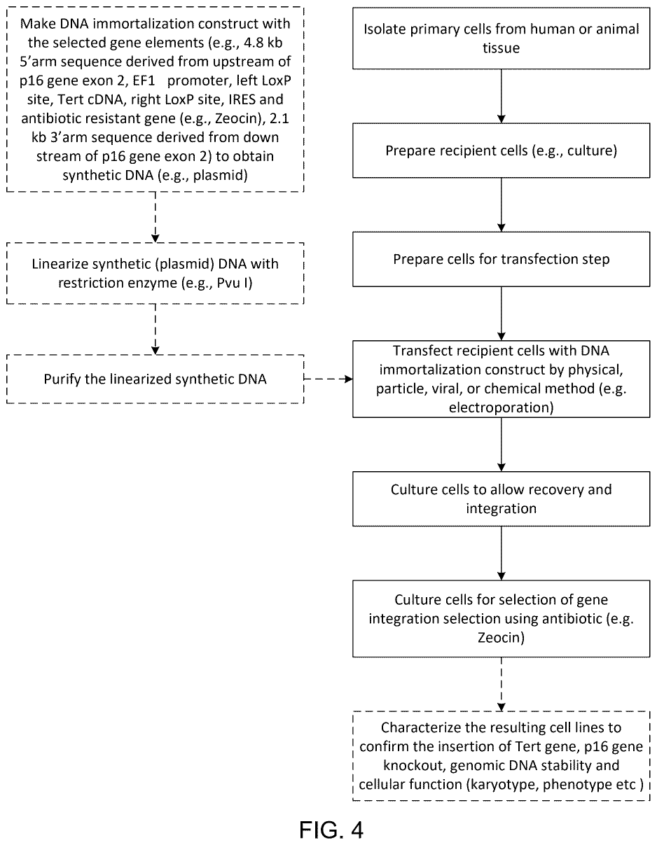

FIG. 4 shows aspects of a process (300) for performing primary cell immortalization using the DNA immortalization construct (200). This process can include steps to generate a stable, immortalized cell line (312) from primary cells (302) isolated from human or animal tissue. The process can begin with the construction of the DNA immortalization construct (200), a step that involves assembling the various genetic elements into a functional unit capable of targeted gene insertion and regulated gene expression. This step involves providing the DNA sequences, including the 5′ arm (202), EF1α promoter (204), first LoxP sequence (206), human TERT gene (208), second LoxP sequence (210), IRES sequence (212), antibiotic resistance gene (214), SV40 poly-A signal (216), and 3′ arm (218). These components can be obtained through PCR amplification from existing sources or chemically synthesized using commercially available oligonucleotide synthesis services. The individual components are then sequentially connected using molecular cloning techniques, such as restriction enzyme digestion and ligation, to produce the complete construct. This construct can be cloned into a suitable vector backbone, such as a plasmid, for propagation and manipulation in bacterial systems. The assembled DNA construct (200) can optionally be linearized, for instance by digesting the plasmid with a restriction enzyme, to facilitate integration into the host genome during transfection. Following construct preparation, primary cells (302) are isolated from human or animal tissue using appropriate techniques, including enzymatic digestion, mechanical dissociation, or cell sorting. The isolated primary cells are then cultured in a specialized medium formulated to support their growth and proliferation, generating a sufficient number of recipient cells (304) for transfection. The recipient cells (304) are then transfected with the DNA immortalization construct (200) using an appropriate method, such as electroporation or lipofection. The transfection method is optimized for the specific cell type and the characteristics of the DNA construct to ensure efficient gene transfer and cell viability. The transfected cells (306) are then cultured in a selective medium (308) containing an antibiotic (310) to which the antibiotic resistance gene (214) within the construct confers resistance. This selection process eliminates non-transfected cells and allows for the expansion of the population of cells carrying the integrated construct, including the hTERT gene (208) for extended lifespan. The transfected cells (306) that survive antibiotic selection are isolated to generate the immortalized cell line (312). This isolation can be achieved through methods such as limiting dilution cloning, ensuring a pure and homogenous cell population. The resulting immortalized cell line (312) is characterized to confirm the successful integration and expression of the hTERT gene, and to verify that the cells retain the desired phenotypic and functional characteristics of their primary cell counterparts. This characterization may include techniques such as PCR analysis, Southern blotting, karyotyping, and functional assays relevant to the cell type and intended application. This comprehensive approach, combining molecular cloning techniques, cell culture methods, and rigorous characterization, ensures the generation of reliable and reproducible immortalized cell lines for various research, development, and commercial applications.

Conventional approaches to achieving cell immortalization often rely on introducing viral genes or activating endogenous oncogenes to overcome the limitations of replicative senescence. This, however, frequently comes at the cost of disrupting cellular function and genomic stability. The random integration of viral vectors and the pleiotropic effects of oncogenes can introduce unpredictable changes to the cellular genome, potentially altering gene expression patterns, promoting tumorigenesis, and disrupting normal cellular processes. The DNA immortalization construct (200) described herein takes a different approach by using targeted gene insertion and homologous recombination to achieve controlled and precise modification of the cellular genome. By incorporating homologous arms derived from the CDKN2A gene, the construct specifically targets the integration of the hTERT gene at the CDKN2A locus, minimizing the risk of random insertion and preserving the genetic integrity of the immortalized cell line. This targeted approach, coupled with the use of a strong and constitutive promoter, such as the EF1α promoter, ensures robust and consistent expression of hTERT, leading to efficient telomere maintenance and extended cellular lifespan. Furthermore, the inclusion of LoxP sites flanking the hTERT gene allows for conditional removal of the gene by Cre recombinase, providing a level of control over hTERT expression not typically found in conventional methods. This conditional regulation enables researchers to study the specific effects of hTERT expression or to revert the cells to a non-immortalized state for specific applications, such as cellular differentiation. This targeted, controlled, and reversible approach to immortalization represents a departure from the often disruptive and unpredictable nature of traditional methods, offering a more refined and adaptable tool for generating stable, functionally representative cell lines for a wide range of research and commercial endeavors.

The articles and processes herein are illustrated further by the following Examples, which are non-limiting.

### Example 1

### Immortalization of Human Primary CD8+ T Cells by Inserting a Single Copy of Human Telomerase Reverse Transcriptase Via CRISPR/Cas9

This examples describes use of a CRISPR/Cas9 system to replace a single copy of the exon 2 of the cell cycle inhibitor gene CDKN2A (encoding p16 and p14 proteins) with a single copy of human telomerase reverse transcriptase (hTERT) to immortalize human primary CD8 T cells (hCD8 T-TERT). By using Cas9 protein and low donor DNA copies/cell, we successfully immortalized hCD8 T cells with a single copy of hTERT transgene, which also avoided uncontrolled insertion of Cas9 gene and guide RNA vector. Human primary CD8+ cells were immortalized and expanded more than 2.6×10⁷ times. Characterization of the cells revealed that the immortalized CD8 T-TERT cells retained most of the cell surface markers and normal karyotype. The CD8 T-TERT cells also retained the dependence of IL-2 and CD3/CD28 activator for survival and expansion.

The Jurkat cell line has been used as normal T cell line and has generated knowledge such as T cell signaling pathways. However, the use of Jurkat cells as a T cell model has decreased in the recent years because it is realized that Jurkat cells are quite abnormal compared to the normal T cells in many ways. Nevertheless, T cell lines are still playing roles in research fields such as cell therapy and immune checkpoints to develop therapeutic products for variety of disease. Such applications include drug testing and disease modeling and so on.

Immortalized normal human T cell lines are useful for basic research and clinical product development. However, many immortalization methods, including SV40 large T antigen, oncogene transfection, and virus all generate abnormality of karyotype and phenotype. Telomerase reverse transcriptase (TERT) gene overexpression is considered the immortalization method that changes the karyotype and phenotype the least. However, the uncontrolled insertion of the TERT transgene can cause chromosome instability of the immortalized cell lines. Several methods have been used to immortalize cell lines in general. One of the widely used methods is transfection of SV40 large T-antigen, which has immortalized many different types of cell lines, such as kidney cell line HEK293T, chondrocyte cell line. Due to the capability of binding to transcription co-factor p300 and CBP, and perturbation of retinoblastoma and p53 tumor suppressor proteins, SV40 large T-antigen immortalized cells often have cancer transformation property. Oncogene transfection was also proved to be efficient for immortalizing human T cell lines from cancer patients or healthy donor. But whether the immortalized cell lines had any chromosome abnormality or mutations were not known. Epstein Bar Virus (EBV) can infect the T cells in vivo and immortalize T cells in vivo. Therefore, some immortalized T cells lines were isolated and established from the EBV infected patients. Human TERT over expression became a widely used method. First, hTERT immortalized cell lines did not lead to tumor formation in immune deficient nude mice, in contrast, SV40 immortalized cell lines showed tumorigenicity. Second, TERT immortalized cell lines showed relative chromosome stability and retained the expression of phenotypic markers. Human T cells were also reported to be immortalized by TERT over expression.

Despite the advantages of TERT over expression immortalization method, most of the delivery method for the TERT transgene utilize retrovirus, lentivirus, or plasmids. These methods all have one major disadvantage resulting in uncontrolled insertion of the transgenes. Uncontrolled insertions may cause insertional mutation or chromosome instability. Recently, some researchers used a new strategy of replacing CDKN2A (p16) exon 2 with TERT transgene by CRISPR/Cas9 technology, which can over express human TERT and inactivate the tumor suppressor gene CDKN2A (p16). An immortalized human prostate epithelial cell line were reported, but the immortalized cell lines had chromosome loss and gain, wherein the researchers used a lentivirus vector for CRISPR/Cas9 and guide RNA delivery, which may cause the consistent expression of CRISPR/Cas9 and guide RNA in the cell progenies and eventually lead to the chromosome abnormality.

In this examples, we over-expressed TERT and CDKN2A inactivation to immortalize human primary T cells. We used CRISPR/Cas9 with conditions to ideally insert inert single copies of the transgene at a specific gene locus minimizing the incidence of off target DNA changes. The immortalized human primary T cell line exhibited a phenotype consistent with of primary human T cells.

### Establishment of Immortalized Normal CD8+ T Cell Line

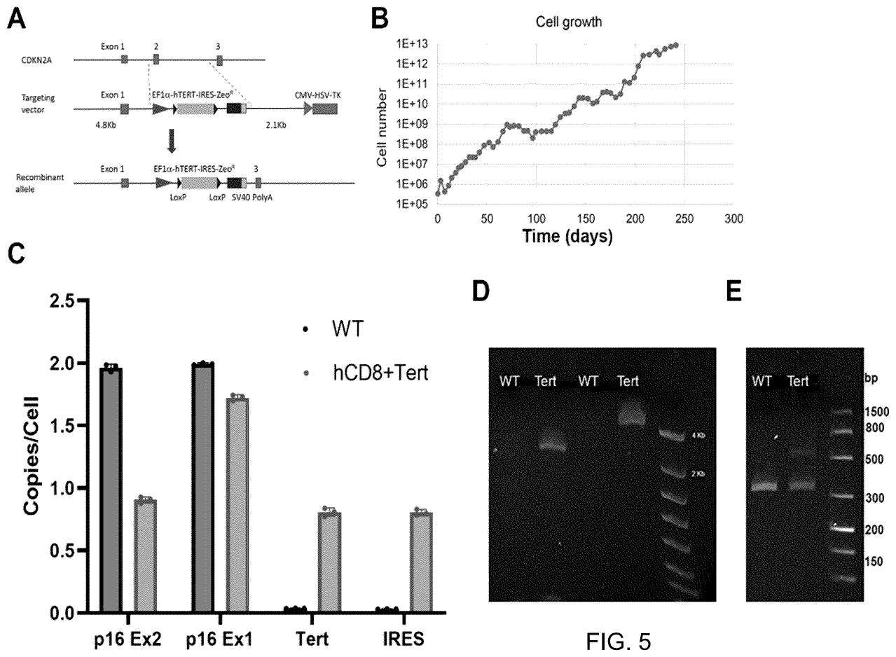

The electroporated CD8+ T cells were cultured in T cell expansion medium with CD3/CD28 activator and IL-2 for all the passages and were selected with 5 μg/mL of Zeocin started from second passage and for over 10 passages. Mock electroporated CD8+ T cells were most killed after 3 passages with Zeocin (data not shown). The live cell number from electroporated cells was declined initially and recovered in the next passage. After 52 passages, the live cells expanded around 3×10⁷ times (FIG. 5B). This data indicated that the CD8 T cells were immortalized. To confirm whether hTERT transgene was incorporated into CDKN2A (p16) gene exon 2, we first used ddPCR to detect the copy number of hTERT transgene per cell. The copy number of the transgene was calculated by the ratio of target gene and reference gene multiply by 2. We found that there were 1 copy of CDKN2A (p16) gene exon2 and 2 copies exon 1 (exon I was a part of 5′ recombinant arm of the donor DNA) in the immortalized CD8 T cells, and there were 2 copies of both p16 gene exon 1 and exon 2 in the WT CD8 T cells (FIG. 5C). One copy of hTERT transgene and IRES were also found in the immortalized CD8 T cells. This data indicated that the targeting donor DNA contained hTERT and Zeocin resistant genes was successfully replaced one copy of p16 gene exon 2. To further confirm the donor DNA was incorporated in the CDKN2A (p16) gene exon 2 as expected, we used 2 pairs of primers to detect the recombinant CDKN2A (p16 allele. Both pairs of primers had the forward primers located in the SV40 polyA signal of the transgene, and reverse primers located 3′ to the 3′ recombinant arm of donor DNA. Both pairs of primers were able to detect the recombinant allele (FIG. 1D). Since we detected 1 copy of hTERT transgene and still had 1 copy of CDKN2A (p16) gene exon 2, the CDKN2A (p16) exon 2 knockout was heterozygous. To test this, we designed primers that can only detect WT CDKN2A (p16) allele and only detect p16 exon 2 knockout allele, namely forward primers only located in the replaced CDKN2A (p16) gene fragment or only located in the hTERT transgene cassette. The PCR data showed that both WT allele and knockout allele were detected (FIG. 5E), which confirmed that only 1 copy of hTERT transgene replaced the CDKN2A (p16) gene exon 2. We call this cell line hCD8⁺ T-TERT hereafter.

TERT gene expression was elevated in hCD8+T-TERT cells.

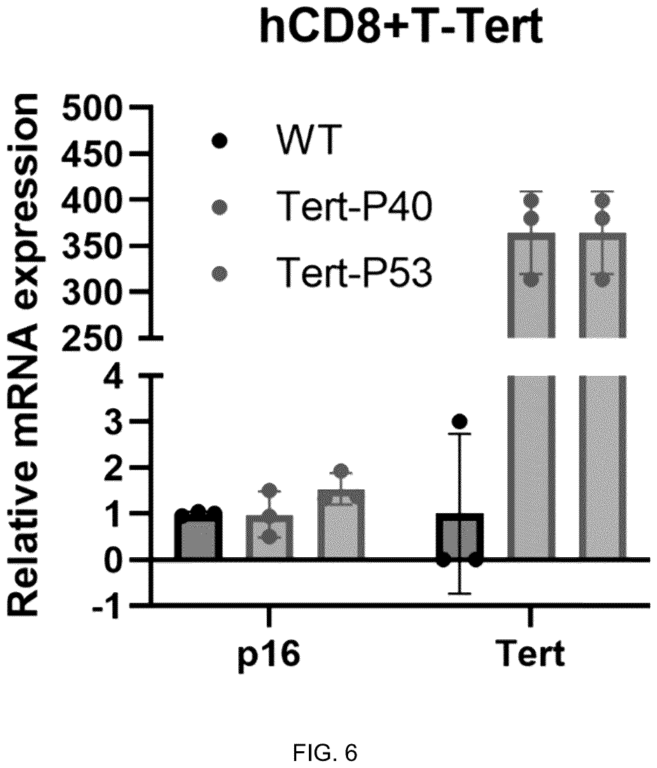

HCD8+T-TERT cells were cultured with Zeocin for all the passages and were resistant to Zeocin, which indicated that the Zeocin resistant gene was expressed in the cells. The hTERT gene was designed at the upstream of Zeocin resistant gene. To test whether hTERT transgene was expressed in hCD8⁺ T-TERT cells, we isolated total RNA from 2 different passages of the cells (passage 40 and passage 53) and control WT CD8⁺ T cells. Direct RT-ddPCR data revealed that hTERT gene expression was over 350 folds elevated in the immortalized cells, but it was minimally detected in WT CD8⁺ T cells (FIG. 6). This data indicated that hCD8⁺ T-TERT cells had the hTERT transgene replaced CDKN2A (p16) exon 2 locus, and the TERT transgene was highly expressed. Since there was one copy of CDKN2A (p16) gene exon 2 was replaced with hTERT transgene, we asked whether p16 mRNA expression in the hCD8⁺ T-TERT cells was reduced compared to WT cells. DdPCR data revealed that the p16 mRNA expression remained almost same as compared with WT CD8⁺ T cells (FIG. 6).

HCD8+T-TERT cells exhibited partial normal karyotype at high passages.

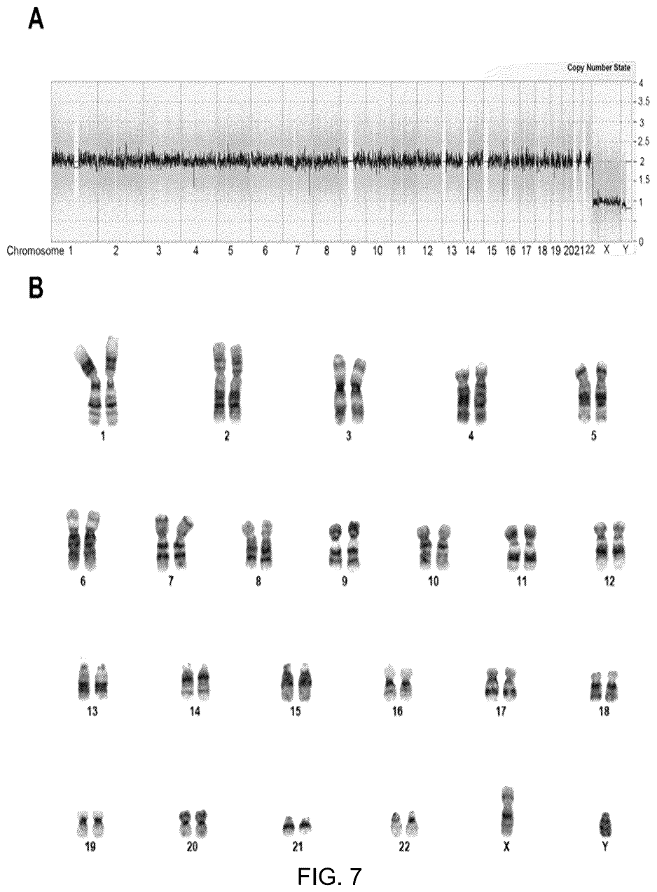

One of the goals of this immortalization method was to have very little alteration of the genome of T cells. With this minimal alteration, the hCD8⁺ T-TERT cell line retained normal karyotype at high passages. The hCD8⁺ T-TERT cells were analyzed by 2 different methods at passage 47. First, Karyostat Karyotyping (microarray analysis, ThermoFisher Scientific) data showed that there were 23 pairs of chromosomes, and no obvious chromosome loss and gain was observed (FIG. 7A). Cytogenic karyotyping results also showed that at least 45% (9 out of 20) of the cells had normal 23 pairs of chromosomes and no obvious chromosome loss and gain (FIG. 7B) (Karyologic, NC). There were some small alterations observed such as deletion of chromosome q11.1 (5 out of 20), translocation of small part of chromosome 10 and 13 (2 out of 20). But chromosome alterations were not observed in Karyostat⁺ analysis. This data indicated that our immortalization method largely retained normal karyotype as WT CD8⁺ T cells.

HCD8+T-TERT cells retained the dependence of IL-2 and CD3/CD28 activator for survival and expansion.

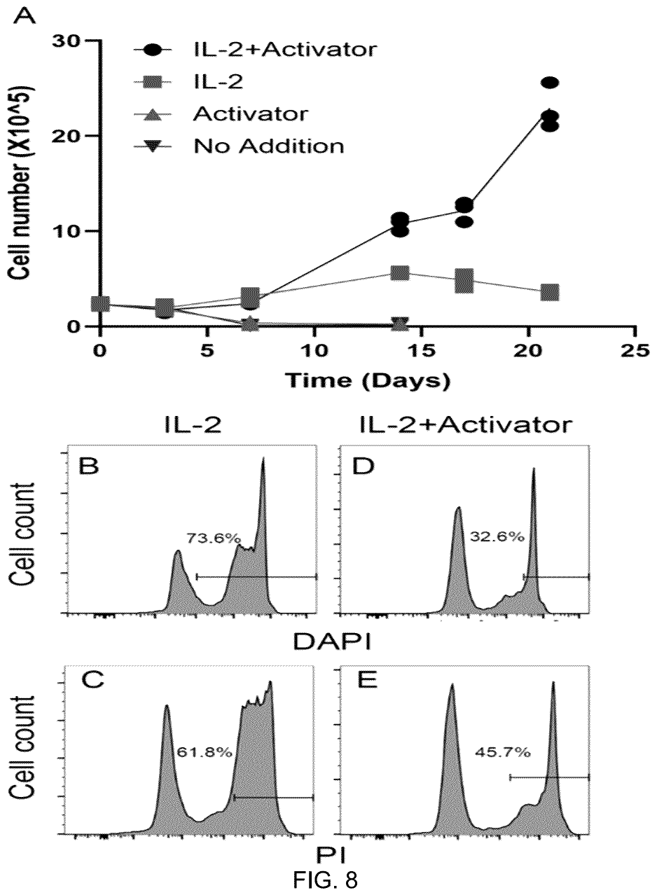

To determine whether the immortalized hCD8+T-TERT cell line retained the dependence of IL-2 and CD3/CD28 activator for survival and expansion as the parental cells, we cultured the immortalized T cells in medium with or without IL-2 and/or CD3/CD28 activator. As shown in FIG. 8A, the cells started to grow after thawed out from the cryopreserved vial in a week in the medium contained both IL-2 and CD3/CD28 activator. However, the cells only survived and expanded in the first few passages in expansion medium contains IL-2 but no CD3/CD28 activator (FIG. 8A). But in the medium without IL-2 (activator alone or no addition), the cells died in a week (FIG. 4A). We further confirmed that large proportion of the cells were dead detected as DAPI positive (73.6%, FIG. 8B) and PI positive (61.8%, FIG. 8C). The cells cultured with IL-2 and CD3/CD28 activator had much fewer dead cells (32.6% DAPI positive and 45.7% PI positive), which indicated that the immortalized cells retained the dependence of IL-2 and CD3/CD28 activator for in vitro survival and expansion.

HCD8+T-TERT cells retained normal CD8+ T cell phenotypic surface markers.

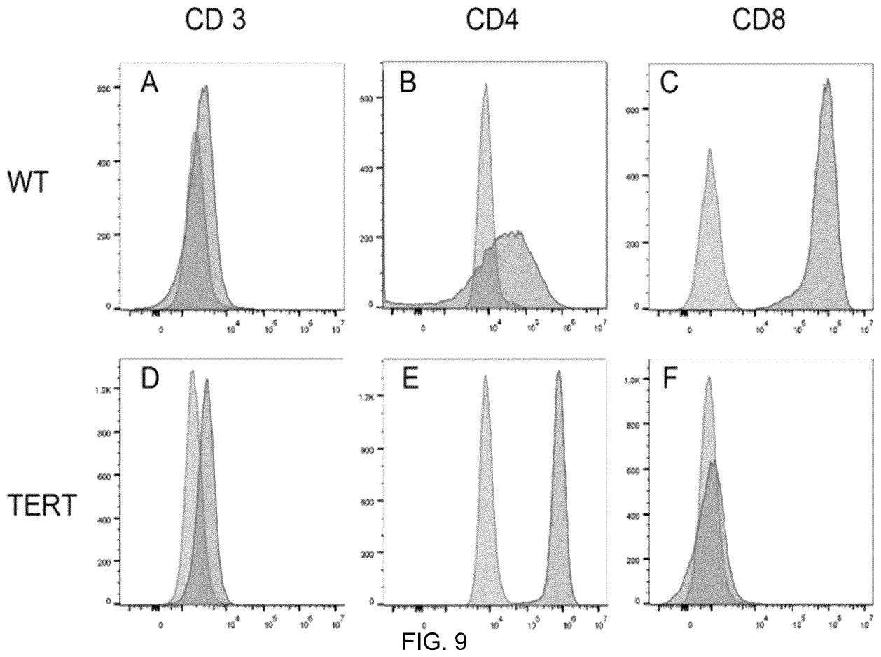

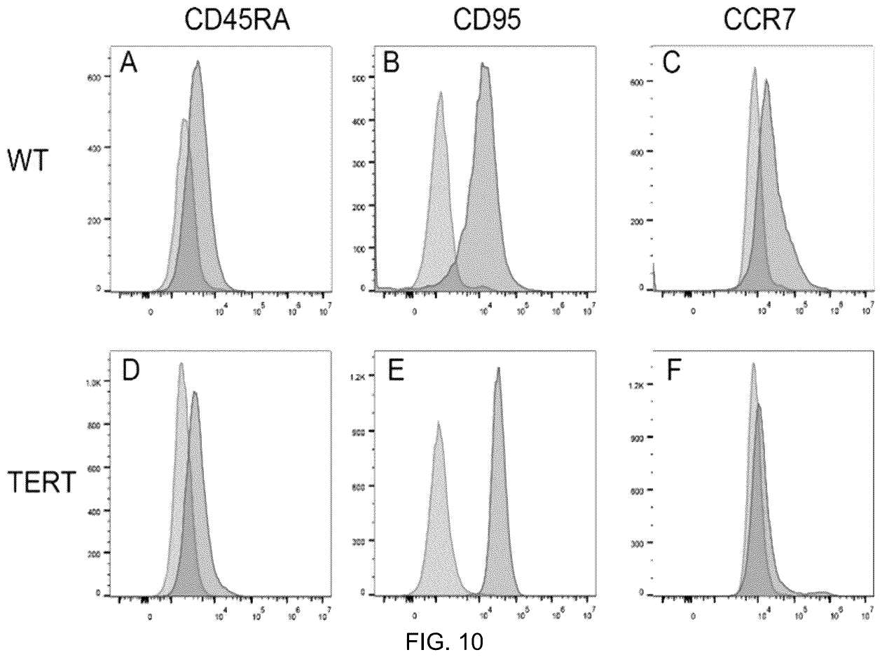

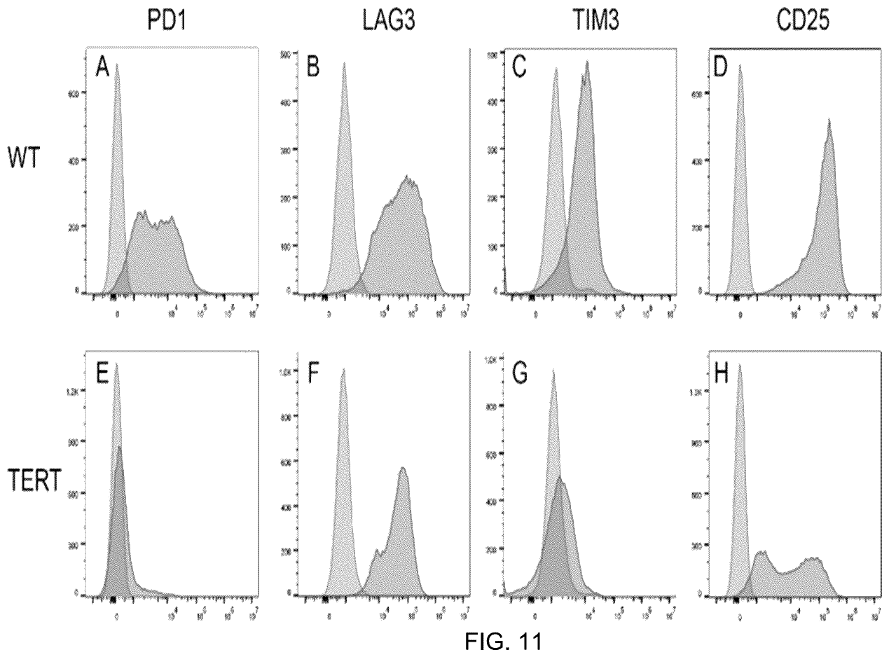

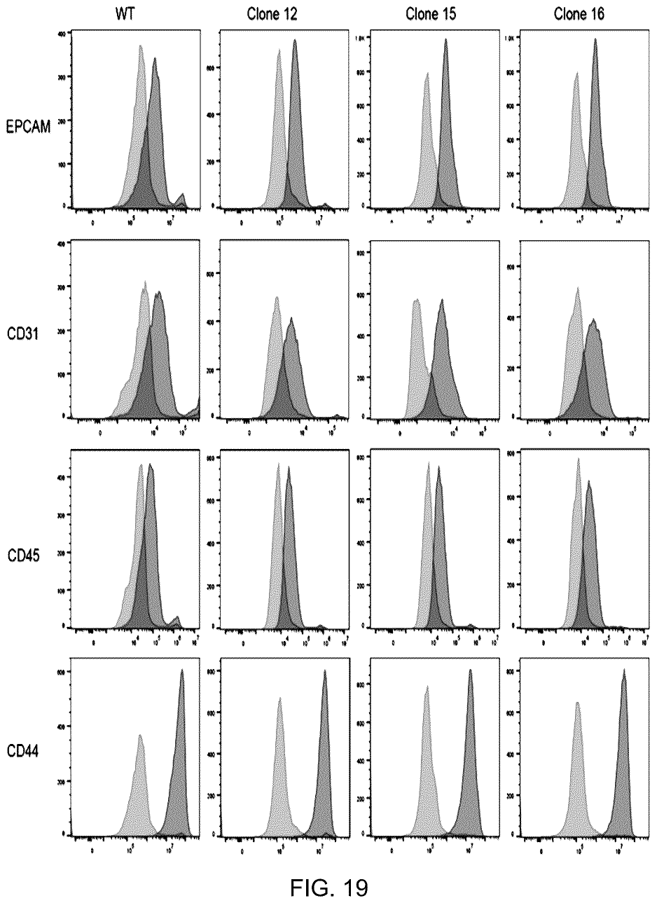

With normal karyotype revealed by 2 different karyotyping methods, we further investigated whether the hCD8+T-TERT cells had retained phenotypic surface markers expression as WT CD8 T cells. We first analyzed the T cell lineage surface markers, CD3, CD4, and CD8. FIG. 9 showed that the hCD8⁺ T-TERT cells retained CD3 and CD4 surface marker expression at P50 as WT CD8 T cells, but lost CD8 surface marker expression. Differentiation markers such as CD45RA and CD95 were also expressed on the hCD8⁺ T-TERT cell surface as WT T cells (FIG. 10A, B, D, E), but CCR7 expression level was lower on the hCD8⁺ T-TERT cells than that of WT T cells (FIG. 10C, F). Equivalent expression of T cell activation surface marker CD25 was observed on hCD8⁺ T-TERT and WT CD8⁺ T cell surface (FIG. 11D, H). Both hCD8⁺ T-TERT and WT T cells had positive expression of exhaustion marker TIM3, LAG3 on the surface (FIG. 11B, C, F, G), but hCD8⁺ T-TERT cells exhibited less PD1 expression than WT T cells (FIG. 11A, E). The presence of the cell surface markers detected by FACS summarized in FIG. 19.

Immortalized T cells have broad applications in both basic research and industry. Jurkat cells has been working as T cell model for decades and generating extensive amount of useful knowledge. However, the limitation of Jurkat cells is now realized simply because they are cancer cells not normal T cells. There had been several T cell lines immortalized by using virus, such as leukemia virus or Herpesvirus. But the virus immortalized, or SV40 large T antigen immortalized cells usually have oncogenic phenotype. Normal T cell lines can fill in the gap that Jurkat cell line was not able to, such as gene and cell therapies. To keep immortalized cell as normal as possible is very challenging, gain and/or deletion of chromosomes were often observed even in the TERT immortalized cell lines. Researchers introduced a method of immortalization human primary epithelial cells with controlled TERT transgene copy number. The authors used CRISPR/Cas9 to introduce TERT transgene in the CDKN2A (p16) exon2 locus, which resulted in over expression of TERT and knockout CDKN2A (p16) at the same time. However, they used Lentivirus vector to deliver the Cas9 and sgRNA, which will integrate into the genome of the immortalized cells. To avoid CRISPR/Cas9 off target, and continuously making double stranded DNA breaks on and off targets, we used CRISPR/Cas9 protein and synthetic sgRNA in this study. To make more precise donor DNA recombination, we used traditional gene knockout strategy of have long 5′ and 3′ recombinant arms. We were able to immortalize human primary CD8⁺ T cells with 500 copies of donor DNA/cell. The single copy of TERT transgene and knockout an allele of CDKN2A (p16) was sufficient to immortalize human CD8⁺ T cells. Moreover, the immortalized T cells largely kept phenotypic surface markers. Although we did see some chromosome small alterations in cytogenic karyotyping, but the Karyostat analysis did not detect these changes. In addition, the finding of the immortalized hCD8⁺ T-TERT cells are still dependent on cytokines and antigen stimulation for proliferation, suggesting that these T cells were not transformed. To our knowledge, this is the first that established a normal human T cell line with single TERT transgene insertion that will be highly useful to for T cell research studies.

### CD8+ T Cells Isolation and Culture

Human primary peripheral blood mononuclear cells (PBMC) were purchased (commercially available from ATCC, Cat #PCS-800-011). The cryopreserved PBMC were thawed in 37° C. water bath. CD8 T cells were isolated immediately from the PBMC by using EasySep Human CD8⁺ T Cell Isolation kit (commercially available from StemCell Technologies, Cat #17953) and following manufacture's protocol. The purified CD8⁺ cells were then cultured in ImmunoCult-XF T cell Expansion Medium (commercially available from StemCell Technologies, Cat #10981) supplied with ImmunoCult Human CD3/CD28 T Cell Activator (commercially available from StemCell Technologies, Cat #10991) and 100 ng/mL of human recombinant IL-2 (commercially available from R&D Systems, Cat #BT-002-100) at 37° C., 5% CO₂. To calculate the cell number expansion, Cell numbers were counted for each passage, the live cell number at each passage was calculated by counted cell number multiple by the passaging dilution factor and the volume of each passage.

### Immortalization Donor DNA Design and Single Guide RNA

Initial experiments utilized the strategy of immortalizing human primary T cells, as previously reported by replace CDKN2 gene with human TERT transgene. To ensure the precise recombination occur at the designed locus, we also adapted the traditional knockout strategy of using 5′ and 3′ recombinant arms. As shown in FIG. 1A, the donor DNA contained 4.8 Kb 5′ recombinant arm from upstream of CDKN2A gene exon 2 (ends at Chr9:21974421). The donor DNA also contained EF1α promoter driven TERT gene followed by internal ribosomal entry site (IRES) and Zeocin resistant gene. We designed the 2.1 Kb 3′ recombinant arm starts from Chr9:21969794 (intron 2) to make the replaced sequence (4.5 Kb) similar size to the transgene (4.6 Kb). At the end of the 3′ recombinant arm, we added a CMV promoter driven Herpes simplex virus thymidine kinase (HSV-Tk) gene for negative selection of random inserted cells. The traditional knockout homologous recombination efficiency was very low for the initial experiments. The second set of experiments used CRISPR/Cas9 to make double strand DNA break to increase the recombinant efficiency. Avoid the uncontrolled insertion of Cas9 expressing sequence, we used Cas9 protein (commercially available from Integrated DNA Technologies, Cat #1081058) instead of Cas9 expressing vectors (to reduce off target cleavage effects). Single guide RNA (sgRNA) sequence was designed on Integrated DNA Technologies website and ordered from Integrated DNA Technologies. The sequence of sgRNA was:

(SEQ. ID NO. 10)
mC*mA*mG*rArUrGrArUrGrCrCrArCrGrCrArCrArArUrGrUrUr
UrUrArGrArGrCrUrArGrArArArUrArGrCrArArGrUrUrArArAr
ArUrArArGrGrCrUrArGrUrCrCrGrUrUrArUrCrArArCrUrUrGr
ArArArArArGrUrGrGrCrArCrCrGrArGrUrCrGrGrUrGrCmU*mU
*mU*rU
(Phosphorothioated 2′-O-methyl RNA bases
are entered as ‘m_*’).

### CD8⁺ T Cell Immortalization

The CD8⁺ T cells were electroporated with Neon Transfection System (commercially available from Life Technologies) by following manufacture's protocol. Briefly, after the purified CD8 T cells were cultured in expansion medium for 2-3 days, the cells were spin down at 250×g for 5 min at room temperature and washed once with PBS without Ca²⁺ and Mg²⁺. The cells were then resuspended in Buffer R at the concentration of 3×10⁶/mL. In a microcentrifuge tube, 100 μL of cells were mixed with 0.7 μL of prepared RNP (mixture of Cas9 protein and guide RNA) and 500 copies/cell donor DNA. The mixture of cells and RNP was loaded into the electroporation tip and electroporated at 1600 v 10 ms for 3 pulses. The cells were then cultured in 0.5 mL T cell expansion medium in 24-well plate at 37° C., 5% CO₂ for 2 days. The cells were passaged and further cultured in T cell expansion medium supplied with 5 μg/mL Zeocin for selection of resistant cells. Genomic DNA was extracted from the cells at the indicated passage for charactering the copy number of transgene and CDKN2A (p16) gene.

### Genomic DNA Extraction and PCR

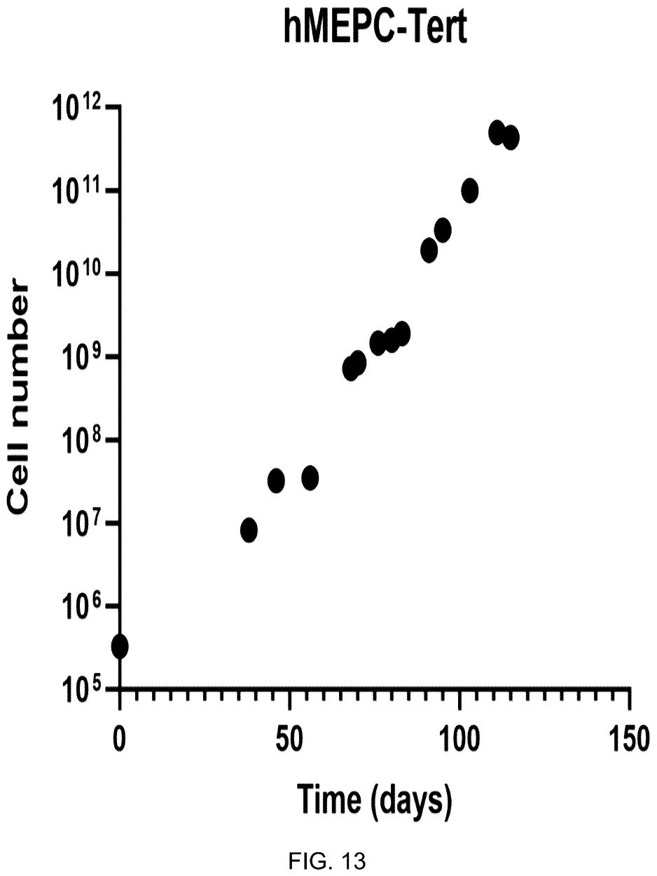

Genomic DNA was extracted from the immortalized cells by using Zymo Quick-DNA kit (commercially available from Zymo Research, Cat #D3025) and following manufacture's protocol. Around 1 ng of genomic DNA was used as template for amplifying recombinant CDKN2A (p16) allele in a polymerase chain reaction (PCR) by using PCR kit (commercially available from Takara Bio, Cat #R050A). The sequence of the primers is listed in Table 1 as a summary of cell surface markers by FACS. The primers design is shown in FIG. 13. For primers can only detect WT allele, Forward primer (P1) was designed in the region of replaced p16 gene, and reverse primer (common primer) (P2) was designed in the 3′ recombinant arm, and the forward primer can only detect KO allele was designed in the transgene region (P3). Two pairs of primers can detect recombinant allele were designed. Both forward primers were designed in the transgene region (P4 and P5) and the reverse primers were designed downstream of 3′ recombinant arm (P6 and P7). These 2 pairs of primers will only amplify from correctly inserted recombinant allele, thus to confirm that the transgene was inserted in the right locus. The amplification of PCR products was visualized in a FlashGel (commercially available from Lonza, Cat #57023).

| Antigen | WT | T-TERT |
|---|---|---|
| CD3 | + | + |
| CD4 | + | ++ |
| CD8 | +++ | − |
| CD45RA | + | + |
| CD95 | ++ | ++ |
| CCR7 | +/− | +/− |
| PD1 | + | +/− |
| LAG3 | ++ | ++ |
| TIM3 | + | +/− |
| CD25 | + | +++/+ |

### Total RNA Isolation

One million of log growth phase immortalized CD8+ T cells and WT CD8+ T cells were aliquoted into a microcentrifuge tube and spun down at 300×g for 5 min at room temperature. The supernatant was removed from the cells. RNA samples were isolated from the cells by using Qiagen RNeasy kit (Qiagen, Cat #74104) and following the kit protocol.

### Droplet Digital PCR

Copy numbers of the CDKN2A (p16) gene and hTERT transgene were analyzed by droplet digital PCR. Ten to forty nanograms of genomic DNA was used in each reaction. Bio-Rad ddPCR Supermix for Probes (no dUTP) was purchased from Bio-Rad (Cat #186-3024). Primers and probes were designed at NIST and purchased from ThermoFisher Scientific, and the sequences for primers and probes are listed in Table 2. Droplets were generated by using droplet generator (commercially available from Bio-Rad). After PCR amplification, droplets were analyzed on Bio-Rad QX200 droplet reader. The copy number of each target gene was calculated by the ratio of target to single copy reference gene (RPL32) multiply by 2. For relative mRNA expression, ddPCR was performed using 1-Step RT-ddPCR Advanced Kit for Probes (commercially available from Bio-Rad, Cat #1864022) and total RNA as template. GAPDH (commercially available from ThermoFisher, Cat #402869) was used as internal control.

| Gene | Forward primer | Reverse primer | Probe |
|---|---|---|---|
| Recombinant | CCATGACCGAGATC | GTGTCAGAAACGATG | N/A |
| p16 | GGCGAG (SEQ. ID | CTGTCTTC (SEQ. ID | |
| | NO. 11) | NO. 12) | |
| Recombinant | CGAGGAGCAGGAC | TCGGTTGGTTGGGAG | N/A |
| p16-2 | TGAATCG (SEQ. ID | GTTTC (SEQ. ID NO. 14) | |
| | NO. 13) | | |
| WT p16 | GTCTGCTGAAACTG | GAGGGGCCGAGTAAA | N/A |
| | CCAACA (SEQ. ID | GAAGA (SEQ. ID NO. | |
| | NO. 15) | 16) | |
| KO p16 | GACTTCGTGGAGGA | | N/A |
| | CGACTT (SEQ. ID NO. | | |
| | 17) | | |
| TERT | AGCCACGTCTCTAC | CTCATTCAGGGAGGA | GCCGTACATGCGA |
| | CTTGAC (SEQ. ID NO. | GCTCT (SEQ. ID NO. 19) | CAGTTC (SEQ. ID |
| | 18) | | NO. 20) |
| P16 | CTTCCTGGACACGC | CAGGTACCGTGCGAC | CTGAGGAGCTGGG |
| exon2 | TGGT (SEQ. ID NO. | ATC (SEQ. ID NO. 22) | CCATC (SEQ. ID NO. |
| | 21) | | 23) |
| P16 | GGGGAGTTTTCAGA | TGGCTCCTCATTCCTC | AATCACAGACCTC |
| exon 1 | AGGGGT (SEQ. ID | TTCC (SEQ. ID NO. 25) | CTCCTGG (SEQ. ID |
| | NO. 24) | | NO. 26) |
| IRES | CTTGGAATAAGGCC | AAGAAGACAGGGCCA | TCTTTTGGCAATGT |
| | GGTGTG (SEQ. ID | GGTTT (SEQ. ID NO. 28) | GAGGGC (SEQ. ID |
| | NO. 27) | | NO. 29) |

### Flow Cytometry

T cell surface markers were analyzed by fluorescent activated cell sorting (FACS) analysis. Immortalized CD8⁺ T cells after 45 passages and WT CD8⁺ T cells within 6 passages were spined down at 300×g for 5 min at room temperature and washed once with FACS buffer (PBS supplied with 10% FBS). The cells were resuspended in antibodies cocktails mixed in FACS buffer. The conjugated antibodies were all commercially available from BioLegend, CD3-pacific blue (cat #300329), CD4-AlexaFluo-488 (Cat #300519), CD8-PE (Cat #344705), CD25-AlexaFluo-647 (Cat #302617), CD45RA-Pacificblue (Cat #304117), CCR7-AlexaFluo-488 (Cat #353205), CD95-PerCP-Cy5.5 (Cat #305629), PD-1-AlexaFluo-647 (Cat #143721), TIM-3-PerCP-Cy5.5 (Cat #345015), and LAG-3-PE (Cat #369305). The cells were incubated with antibodies cocktail on ice for 1 hr, and then washed with 1 mL FACS buffer. The cells were subjected to Cytoflex flow cytometer after resuspending the cells in 300 μL FACS buffer. For dead cell analysis, the cells were spun down and washed with FACS buffer and resuspended in 300 μL FACS buffer contained 1 μg/mL of 4′,6′-diamidino-2-phenylindole (DAPI) and propidine iodide (PI). The staining of DAPI and PI was analyzed on Cytoflex. The FACS data was analyzed on FlowJo software (commercially available from FlowJo, LLC). Each cell sample cultured in different medium condition without DAPI or PI staining was used for DAPI and PI negative gating.

Various abbreviations used herein include: WT wild type; hTERT human telomerase reverse transcriptase; IRES internal ribosomal entry site; and ddPCR droplet digital polymerase chain reaction.

FIG. 5 shows immortalization of human primary CD8⁺ T cells, wherein: A, schematic view of donor DNA design and knockout p16 exon 2 after recombination. B, growth of immortalized CD8⁺ T-TERT cells. C, copy numbers of p16 exon2, exon 1, TERT transgene and IRES in the immortalized CD8⁺ T-TERT cells. D, recombinant p16 allele was detected in CD8⁺ T-TERT cells (lanes labeled Tert) by 2 sets of PCR primers, but not in the WT CD8⁺ cells (lanes labeled WT). E, PCR detected p16 WT and KO alleles in CD8 T-TERT cells but only detected p16 WT allele in WT CD8⁺ cells.

FIG. 6 shows relative p16 and TERT mRNA expression in hCD8⁺ T-TERT cells. HCD8⁺ T-TERT cells express higher level of TERT and equivalent level of p16 mRNA compared to parental WT T cells.

FIG. 7 shows karyotype of hCD8 T-TERT cells. A, Microarray-based Karyostat⁺ using genomic DNA extracted from 2 million cells showed normal Karyotype of hCD8⁺ T-TERT cells. Y axis indicated the copy number and x axis indicated the chromosome number. B, Cytogenic karyotype assay analyzed 20 metaphase spreads, showed 45% (9 out of 20) of hCD8⁺ T-TERT cells had normal karyotype.

FIG. 8 shows immortalized T cells retained dependence of IL-2 and CD3/CD28 activator. A, hCD8 T-TERT cells growth in the media contained IL-2⁺ CD3/CD28 activator, IL-2, activator, or no addition. B, FACS plot showed the percentage of DAPI positive cells cultured in medium contained IL-2. C, FACS plot showed the percentage of PI positive cells cultured in medium contained IL-2. D, FACS plot showed the percentage of DAPI positive cells cultured in medium contained IL-2 and CD3/CD28 activator. E, FACS plot showed the percentage of PI positive cells cultured in medium contained IL-2 and CD3/CD28 activator.

FIG. 9 shows T cell surface markers expression. A-B, CD3 and CD4 were expressed on portion of WT CD8⁺ T cells. C, CD8 was highly expressed on WT CD8⁺ T cells. D, CD3 was expressed on portion of hCD8⁺ T-TERT cells. E, CD4 was highly expressed on all hCD8⁺ T-TERT cells. F. CD8 was down regulated on hCD8⁺ T-TERT cells surface. Blue indicated isotype control and red indicated antibody staining.

FIG. 10 shows T cell differentiation markers expression. A-F, CD45RA, CD95, and CCR7 all were expressed on both WT CD8⁺ T cells and hCD8⁺ T-TERT cells. Blue indicated isotype control and red indicated antibody staining.

FIG. 11 T shows cell exhaustion and activation surface markers expression. A-C, most of the WT CD8⁺ T cells express PD1, LAG3, and TIM3 at P4. D, T cell activation marker was expressed on all WT CD8⁺ T cells. E, PD1 surface expression was down regulated on hCD8⁺ T-TERT cells. F, LAG3 expression on hCD8⁺ T-TERT cells was equivalent to WT CD8⁺ T cells. G, TIM3 expression was down regulated on hCD8⁺ T-TERT cells. H, T cell activation marker CD25 was expressed on all the hCD8⁺ T-TERT cells. Blue indicated isotype control and red indicated antibody staining.

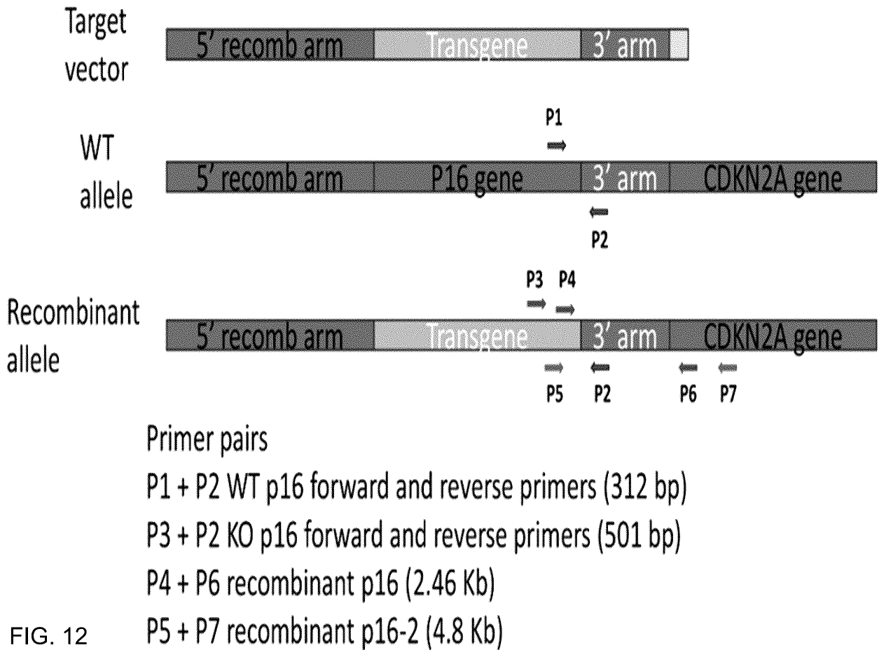

FIG. 12 shows a genotyping primers design.

### Example 2

### Novel Immortalization Method Immortalized Human Primary Mammary Epithelial Cells

Immortalized normal human mammary epithelial cell lines are invaluable tools for basic research such as understanding the mechanisms of breast cancer malignancy and for preclinical research such as development of new treatments for breast cancer. These cell lines can be important for biomedical studies such as senescence, cancer research, drug testing, genetic studies, and modeling different subtypes of breast cancer. The human primary cell immortalization method was applied to the immortalization of human primary mammary epithelial cells (hMEPC).

Immortalization of hMEPC

Primary hMEPCs usually stop dividing and undergo senescence around 10 passages (data not shown). The primary hMEPCs were electroporated with linearized targeting vector and Cas9/sgRNA complex, and cultured for 3 days. The cells were then selected with 5 μg/mL of Zeocin for 3 passages and with 10 μg/mL Zeocin for 10 passages. The mock electroporated cells were killed by Zeocin within 3 passages. The electroporated cells kept growing for at least 50 passages (we cryopreserved the cells at this point) and the cell population expanded more than 3×10⁶ times (FIG. 13, a growth curve of immortalized hMEPC). This data indicated that the hMEPC cells were immortalized.

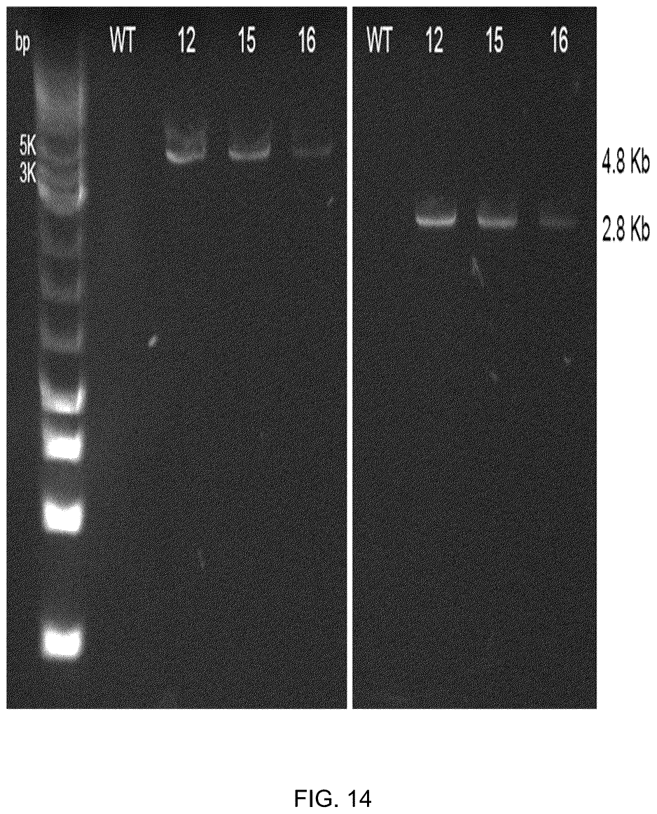

We obtained clonal cell lines, the cell lines were further characterized. We first confirmed that the hTert transgene was inserted in the p16 exon2 locus. FIG. 14 showed that 2 pairs of recombinant allele specific primers (only amplify recombinant allele, not wild type allele, nor target vector) detected all 3 clones. FIG. 14 shows recombinant specific allele primers amplified fragment of DNA only in the recombinant allele from all 3 clones but not WT parental cells.

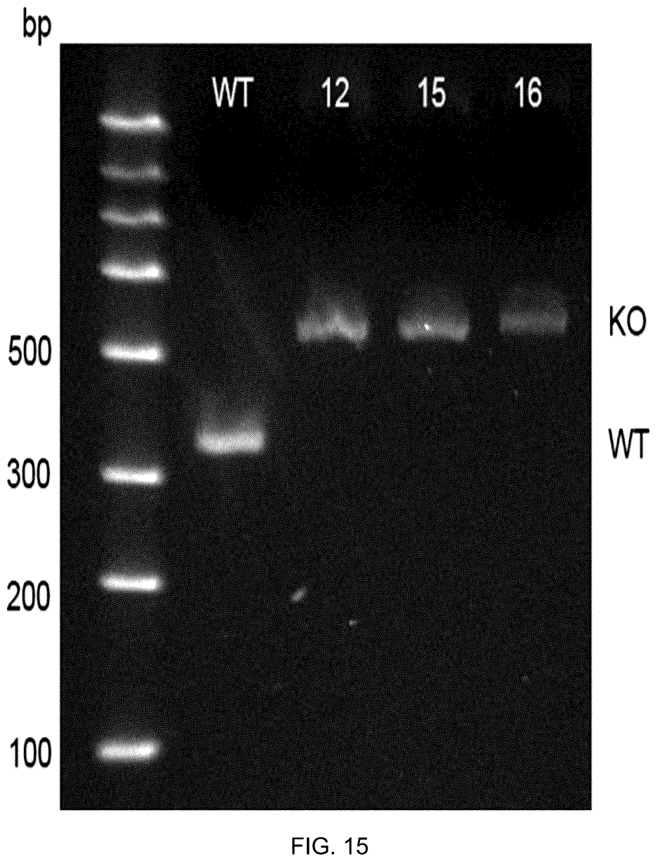

We also used primers that can specifically detect WT allele and primers can only detect p16 exon2 knock allele to analyze how many alleles were replaced by transgene. FIG. 15 showed that all 3 clones were p16 exon2 knockout homozygous. There was only KO allele detected but not the WT allele. FIG. 15 shows results for PCR using WT and KO specific primers that revealed that all 3 clonal cell lines are p16 exon2 KO homozygous.

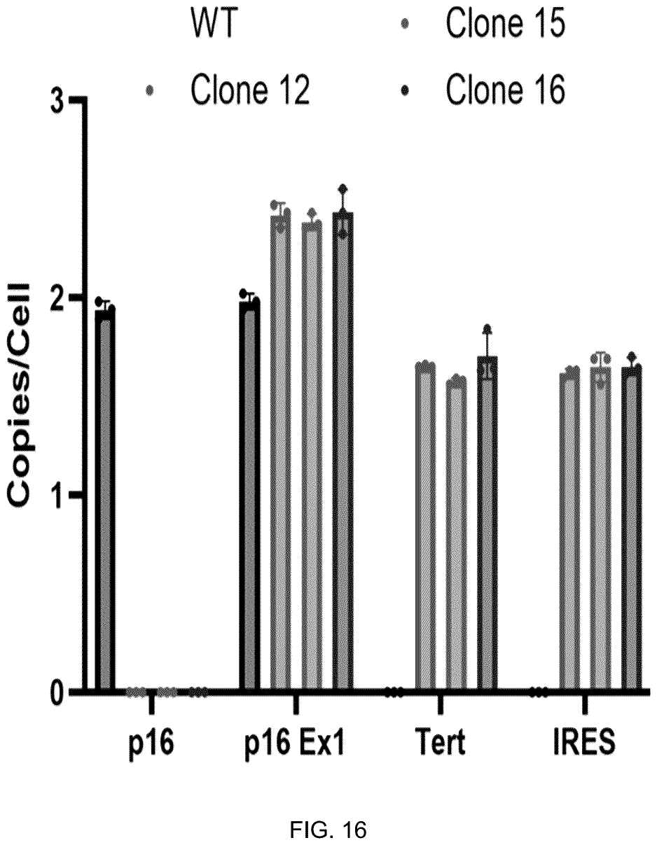

Digital PCR analysis proved that there were 2 copies of transgene (Tert and IRES are part of the transgene) per cell in all 3 clonal cell lines, and there was no p16 exon2 detected, which confirmed that the cell lines were p16 KO homozygous (FIG. 16). P16 exon1 was part of the 5′ recombinant arm, therefore 2 copies of p16 exon1 per cell was expected and the ddPCR data confirmed there were 2 copies of p16 exon1 per cell in the WT parental epithelial cells and all 3 clonal cell lines. FIG. 16 shows data for gene copy numbers detected by ddPCR.

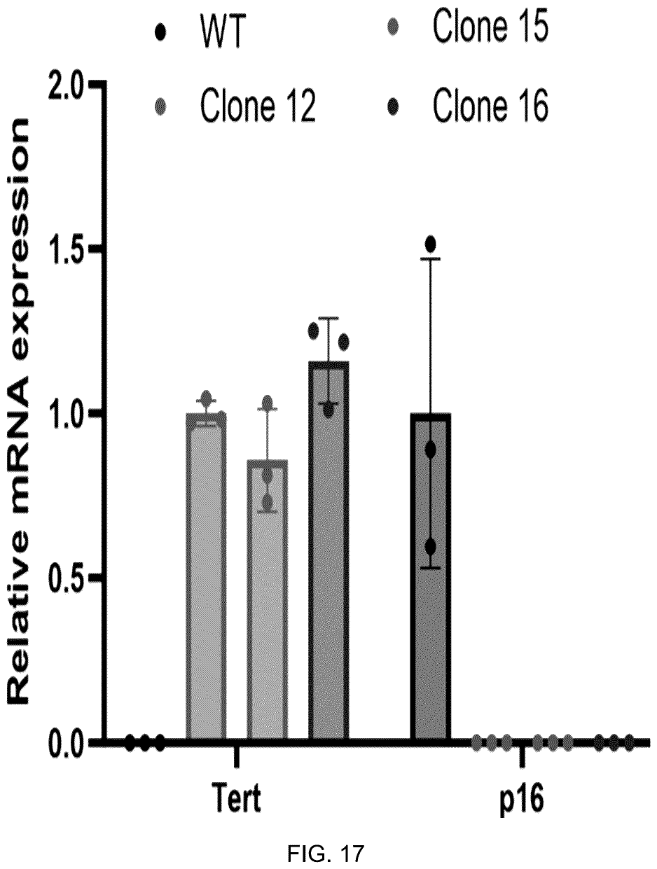

Since the clonal cell lines were p16 KO homozygous, we expected that there would be no p16 mRNA expressed in these cells. We isolated total RNA from the parental WT cells and 3 clonal cell lines. RT-ddPCR revealed that there was no p16 mRNA expressed in the clonal cell lines (FIG. 17). We also confirmed that hTert transgene was expressed in the immortalized clonal cell lines (FIG. 17). FIG. 17 shows Tert and p16 mRNA level expression in parental WT epithelial cells and immortalized clonal cell lines. Tert transgene mRNA expression was detected in the immortalized clonal cell lines but was not detectable in the parental epithelial cells. P16 mRNA was detected in the parental epithelial cells but not detected in the clonal cell lines.

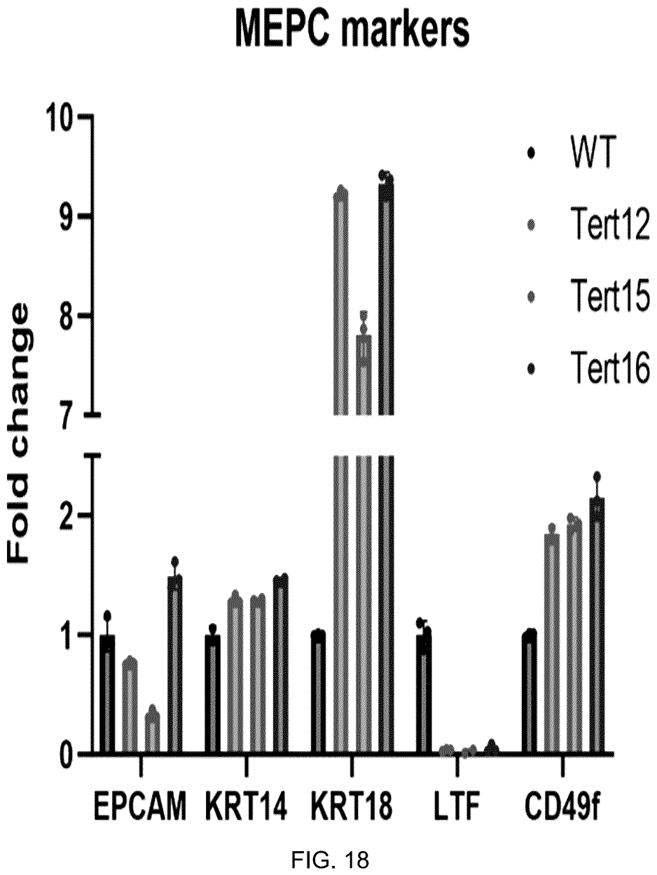

Most of the epithelial cell marker genes were also found expressed on mRNA level in the clonal cell lines. FIG. 18 showed that epithelial cell markers EPCAM, KRT14, KRT18, and CD49f mRNA was detected in the clonal cell lines as well as parental epithelial cells. Only one marker gene LTF was found of low expression in the clonal cell lines. FIG. 18 shows data for epithelial cell marker gene expression on mRNA level by RT-ddPCR.

FACS analysis further revealed that all 3 clonal cell lines expressed equivalent surface markers as parental epithelial cells as shown in FIG. 19. FIG. 19 shows data for FACS analysis of epithelial cell surface markers EPCAM, CD31, CD45, and CD44. All 3 clonal cell lines expressed equivalent surface markers as parental WT epithelial cells (blue isotype control, red antibody staining).

While one or more embodiments have been shown and described, modifications and substitutions may be made thereto without departing from the spirit and scope of the invention. Accordingly, it is to be understood that the present invention has been described by way of illustrations and not limitation. Embodiments herein can be used independently or can be combined.

All ranges disclosed herein are inclusive of the endpoints, and the endpoints are independently combinable with each other. The ranges are continuous and thus contain every value and subset thereof in the range. Unless otherwise stated or contextually inapplicable, all percentages, when expressing a quantity, are weight percentages. The suffix(s) as used herein is intended to include both the singular and the plural of the term that it modifies, thereby including at least one of that term (e.g., the colorant(s) includes at least one colorants). Option, optional, or optionally means that the subsequently described event or circumstance can or cannot occur, and that the description includes instances where the event occurs and instances where it does not. As used herein, combination is inclusive of blends, mixtures, alloys, reaction products, collection of elements, and the like.

As used herein, a combination thereof refers to a combination comprising at least one of the named constituents, components, compounds, or elements, optionally together with one or more of the same class of constituents, components, compounds, or elements.

All references are incorporated herein by reference.

The use of the terms “a,” “an,” and “the” and similar referents in the context of describing the invention (especially in the context of the following claims) are to be construed to cover both the singular and the plural, unless otherwise indicated herein or clearly contradicted by context. It can further be noted that the terms first, second, primary, secondary, and the like herein do not denote any order, quantity, or importance, but rather are used to distinguish one element from another. It will also be understood that, although the terms first, second, etc. are, in some instances, used herein to describe various elements, these elements should not be limited by these terms. For example, a first current could be termed a second current, and, similarly, a second current could be termed a first current, without departing from the scope of the various described embodiments. The first current and the second current are both currents, but they are not the same condition unless explicitly stated as such.

The modifier about used in connection with a quantity is inclusive of the stated value and has the meaning dictated by the context (e.g., it includes the degree of error associated with measurement of the particular quantity). The conjunction or is used to link objects of a list or alternatives and is not disjunctive; rather the elements can be used separately or can be combined together under appropriate circumstances.

### PARTS LIST

DNA immortalization construct 200
5′ arm 202
EF1α promoter sequence 204
first LoxP sequence 206
human TERT gene sequence 208
second LoxP sequence 210
IRES sequence 212
antibiotic resistance gene sequence 214
SV40 poly-A signal sequence 216
3′ arm 218
vector backbone sequence 220
primary cells 302
recipient cells 304
transfected cells 306
medium 308
antibiotic 310
immortalized cell line 312
method 300
method 400

What is claimed is:

1. A DNA immortalization construct (200) comprising: a first nucleic acid sequence (202) derived from the 5′ end of human CDKN2A gene exon 2; an EF1α promoter sequence (204) connected to the first sequence (202); a first LoxP sequence (206) connected to the EF1α promoter sequence (204); a human TERT gene sequence (208) connected to the first LoxP sequence (206); a second LoxP sequence (210) connected to the human TERT gene sequence (208); an internal ribosomal entrance site (IRES) sequence (212) connected to the second LoxP sequence (210); an antibiotic selection gene sequence (214) connected to the IRES sequence (212); an SV40 poly-A signal sequence (216) connected to the antibiotic selection gene sequence (214); and a second nucleic acid sequence (218) connected to the SV40 poly-A signal sequence (216) and derived from the 3′ end of human CDKN2A gene exon 2.

2. The DNA immortalization construct (200) of claim 1, wherein the first sequence (202) comprises 4.8 kilobases.

3. The DNA immortalization construct (200) of claim 1, wherein the antibiotic selection gene sequence (214) is a Zeocin resistance gene sequence.

4. The DNA immortalization construct (200) of claim 1, wherein the second sequence (218) comprises 2.1 kilobases.

5. The DNA immortalization construct (200) of claim 1, further comprising a vector backbone sequence (220) connected to the first sequence (202) and the second sequence (218).

6. The DNA immortalization construct (200) of claim 5, wherein the vector backbone sequence (220) is selected to promote homologous recombination.

7. The DNA immortalization construct (200) of claim 1, wherein the construct (200) is a synthetic plasmid.

8. The DNA immortalization construct (200) of claim 1, wherein the IRES sequence (212) is positioned between the human TERT gene sequence (208) and the antibiotic resistance gene sequence (214).

9. The DNA immortalization construct (200) of claim 1, wherein the first LoxP sequence (206) and the second LoxP sequence (210) allow removal of the human TERT gene sequence (208) by CRE-recombination.

10. The DNA immortalization construct (200) of claim 1, wherein the construct comprises the base sequence of SEQ ID NO: 9.

11. A method (300) for immortalizing primary cells, the method comprising: providing a DNA immortalization construct (200) comprising a first nucleic acid sequence (202) derived from the 5′ end of a human CDKN2A gene exon 2; an EF1α promoter sequence (204) connected to the first sequence (202); a human TERT gene sequence (208) connected to the EF1α promoter sequence (204); an antibiotic resistance gene sequence (214); and a second nucleic acid sequence (218) derived from the 3′ end of the human CDKN2A gene exon 2; isolating primary cells (302) from human tissue; culturing the primary cells (302) to produce recipient cells (304); transfecting the recipient cells (304) with the DNA immortalization construct (200) using homologous recombination to produce transfected cells (306); culturing the transfected cells (306) in a medium (308) comprising an antibiotic (310) to select for transfected cells exhibiting antibiotic resistance; and isolating the transfected cells (306) exhibiting resistance to the antibiotic (310) to produce an immortalized cell line (312).

12. The method of claim 11, further comprising linearizing the DNA immortalization construct (200) prior to transfecting the recipient cells (304).

13. The method of claim 11, wherein linearizing comprises digesting the DNA immortalization construct (200) with a restriction enzyme.

14. The method of claim 13, wherein the restriction enzyme is PvuI.

15. The method of claim 11, wherein transfecting comprises electroporation.

16. The method of claim 11, further comprising: expanding the immortalized cell line (312); and cryopreserving the immortalized cell line (312).

17. The method of claim 11, wherein the primary cells (302) are CD8+ T-cells.

18. The method of claim 11, wherein the immortalized cell line (312) comprises a single copy of the human TERT gene sequence (208) inserted into the CDKN2A gene.

19. The method of claim 18, wherein the immortalized cell line (312) exhibits a decreased expression of the CDKN2A gene.

20. The method of claim 11, wherein the immortalized cell line (312) exhibits a normal karyotype and expresses cell surface markers consistent with the primary cells (302).

21. A method (400) for producing a DNA immortalization construct (200), the method comprising: providing a first nucleic acid sequence (202) comprising about 4.8 kilobases and derived from the 5′ end of a human CDKN2A gene exon 2; providing an EF1α promoter sequence (204); providing a first LoxP sequence (206); providing a human TERT gene sequence (208); providing a second LoxP sequence (210); providing an internal ribosomal entry site (IRES) sequence (212); providing an antibiotic resistance gene sequence (214); providing an SV40 poly-A signal sequence (216); providing a second nucleic acid sequence (218) comprising about 2.1 kilobases and derived from the 3′ end of the human CDKN2A gene exon 2; and sequentially connecting the first nucleic acid sequence (202), the EF1α promoter sequence (204), the first LoxP sequence (206), the human TERT gene sequence (208), the second LoxP sequence (210), the IRES sequence (212), the antibiotic resistance gene sequence (214), the SV40 poly-A signal sequence (216), and the second nucleic acid sequence (218) to produce the DNA immortalization construct (200).

22. The method of claim 21, wherein providing the first nucleic acid sequence (202) comprises chemically synthesizing the sequence.

23. The method of claim 21, wherein providing the second nucleic acid sequence (218) comprises chemically synthesizing the sequence.

24. The method of claim 21, wherein providing the EF1α promoter sequence (204) comprises obtaining the sequence from a preexisting vector.

25. The method of claim 21, wherein providing the human TERT gene sequence (208) comprises synthesizing the sequence from an RNA template.

26. The method of claim 21, wherein the antibiotic resistance gene sequence (214) confers resistance to Zeocin.

27. The method of claim 21, further comprising inserting the sequentially connected sequences into a vector backbone (220).

28. The method of claim 27, wherein the vector backbone (220) is a plasmid.

29. The method of claim 28, further comprising linearizing the plasmid comprising the DNA immortalization construct (200).

30. The method of claim 29, wherein linearizing comprises digesting the plasmid with a restriction endonuclease.


SEQUENCE LISTING (29 sequences)

SEQ ID NO: 1 - DNA, 4833 bp, unidentified
  source 1..4833: mol_type = other DNA
ggatccggaataagtctaaatgaatcactttcagttttcctaaacttctatgcctttgcacatcctcttacctctgcctagaatatctttctccttcttttccatctttaaactctcacatcattcttcaagactgggatcagctctcagcatccggaagcctttgcctactagagacaaatgagaatgagtttggtcaccttttcattttcttgtatcattctgtgctttattttgctcttctaagagcgttacatgcttcatttaatccctaaacaactgtttgaggcaagtacagttattatcctaatcatgcaaatgagaaaacagaggcccagacatgttgagtaactttgataaaagttaaagaaccaataagtggaacagttgaggtttgaaccctggcagtctgactgtagagatactatgtttgacctactcccctctgcccccaccccatgtctgcccttagtttctgagcttgttgaatgaatgaacaggtggtagtctttttttgttataagactgatcagaattaagacaggtttaaatttcacgtgtagaattttcaaaactgcaaaggcagtgcaaatctaaaaaaagaatggcattctcaggaaagaggaaaagtaagtgtgagaataataataacaataaccaacaaactttagtaaatttagtaaatgtagtaaatttttacattaaaagcttttggacatacattatcatattttatggccacatgaaatatattataatcccattttgcacataggaaatctgagactggcataaggagcacagagatccaggactttatattttcattcttctaggattttgcacctcaggtcgatatgtatgagtaaactgggagtataatgggctctttaacagaaaaactaggaaagttttcccactattattaattatttacataatatttttttaattttattattatttatactttaagttttagagtacatgtgcacaatgtgcaggtttgttacatatgtatacatgtgccatgttggtgtgctgcacccatcaactcatcatttagcattaggtatatctcctaatgctatccctcccccctcccccctacataagatttataatggataatggacttcaatttctagagcaaaatggccccacccaaggatgccataatccttccagagctctactgcaagatatgagatatacatatctaaaacttgttcttggtatttccaaagcagtcaacttttacacctgtttataatgcatccaaatgttgtttttatatggttgcatctcccatcttcttcaccaatagctatatatatttttcacaagagctgaaagagttcttgatgtaggaatccatggtagagtttcagagaaatccctgaattcactgaaagttttatctagaaatacatgtgcaagtgaacacatcttttttaaaaaaaatcattacctactttcttttttgagaagaaggtatttatttcaacagactcttgaaggagcctactcttcccactctcccacccccattaagaaccactgtaggccgggcacgatggctcatgcctgtaatcccagcactttgggaggctaaggtgggtggatcacctgaggtcaggagttcgagacaagcctagccaacatagtgaaaccccgtctctactaataatacaaaaattagctgggtatggcagcatgtgcctgtaatcccagctactcgggaggctgaggcaggagaattgctcgaacccgggaggcggaggttgcagtgaaccgagagagatcgtgcggtgccatttcactccagcctgggcaacagagcgaaactccatctcaaaaaaacacacaaaacaaacaaacaaaaagaaagaaccattgtattagtgatggaaatgtgttccctccctcccatcctggcaaccactttcttcctcctccatcataaaatatcttaaactaaactaaaataattttatttatcgatagtttgaattttccctatcattgctacacagctaattgagaggtaccccgaggaaaatataaatggtacagtaatgcattgtagattttaataacatacttgacatcccaaattgttttcattggcttcattttaaaaactacatgttttaaaatcaagcagacactaaaagtacaagatatactgggtctacaaggtttaagtcaaccagggattgaaatataacttttaaacagagctggattatccagtaggcagattaagcatgtgcttaaggcatcagcaaagtctgagcaatccattttttaaaacgtagtacatgtttttgataagcttaaaaagtagtagtcacaggaaaaattagaacttttacctccttgcgcttgttatactctttagtgctgtttaacttttctttgtaagtgagggtggtggagggtgcccataatcttttcagggagtaagttcttcttggtctttctttctttctttctttctttttttcttgagaccaagtttcgctcttgtctcccaggctggagtgcaatggcgcgatctcggctcactgcaacctccgccttctcctgggttcaagcgattctcctacatcagcctccgagtagctgggattacaggcatgcgccaccaagccccgctaattttgtattttttagtagagacagggtttcgccatgttggtcaggcttgtctcgaactcctggcctcaggtgatccgcctgtctcggcctcccagaatgctgggattatagacgtgagccaccgcatccggactttccttttatgtaatagtgataattctatccaaagcatttttttttttttttttgagtcggagtctcattctgtcacccaggctggagggtggtggcgcgatctcggcttactgcaacctctgcctcccgggttcaagcgattctcctgcctcagcctcctgagtagctggaattacacacgtgcgccaccatggccagctaatttttgtatttttagtagagacggggtgtcaccattttggccaagctggcctcgaactcctgacctcaggtgatctgcccgcctcggcttcccaaagtgctgggattacaggtgtgagccaccgcgtcctgctccaaagcattttctttctatgcctcaaaacaagattgcaagccagtcctcaaagcggataattcaagagctaacaggtattagcttaggatgtgtggcactgttcttaaggcttatatgtattaatacatcatttaaactcacaacaacccctataaagcagggggcactcatattcccttccccctttataattacgaaaaatgcaaggtattttcagtaggaaagagaaatgtgagaagtgtgaaggagacaggacagtatttgaagctggtctttggatcactgtgcaactctgcttctagaacactgagcactttttctggtctaggaattatgactttgagaatggagtccgtccttccaatgactccctccccattttcctatctgcctacaggcagaattctcccccgtccgtattaaataaacctcatcttttcagagtctgctcttataccaggcaatgtacacgtctgagaaacccttgccccagacagccgttttacacgcaggaggggaaggggaggggaaggagagagcagtccgactctccaaaaggaatcctttgaactagggtttctgacttagtgaaccccgcgctcctgaaaatcaagggttgagggggtagggggacactttctagtcgtacaggtgatttcgattctcggtggggctctcacaactaggaaagaatagttttgctttttcttatgattaaaagaagaagccatactttccctatgacaccaaacaccccgattcaatttggcagttaggaaggttgtatcgcggaggaaggaaacggggcgggggcggatttctttttaacagagtgaacgcactcaaacacgcctttgctggcaggcgggggagcgcggctgggagcagggaggccggagggcggtgtggggggcaggtggggaggagcccagtcctccttccttgccaacgctggctctggcgagggctgcttccggctggtgcccccgggggagacccaacctggggcgacttcaggggtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtgggtagagggtctgcagcgggagcaggggatggcgggcgactctggaggacgaagtttgcaggggaattggaatcaggtagcgcttcgattctccggaaaaaggggaggcttcctggggagttttcagaaggggtttgtaatcacagacctcctcctggcgacgccctgggggcttgggaagccaaggaagaggaatgaggagccacgcgcgtacagatctctcgaatgctgagaagatctgaaggggggaacatatttgtattagatggaagtatgc

SEQ ID NO: 2 - DNA, 1194 bp, unidentified
  source 1..1194: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattc

SEQ ID NO: 3 - DNA, 34 bp, unidentified
  source 1..34: mol_type = other DNA
ataacttcgtatagcatacattatacgaagttat

SEQ ID NO: 4 - DNA, 3419 bp, unidentified
  source 1..3419: mol_type = other DNA
acccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatgaccaagctt

SEQ ID NO: 5 - DNA, 576 bp, unidentified
  source 1..576: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataata

SEQ ID NO: 6 - DNA, 401 bp, unidentified
  source 1..401: mol_type = other DNA
gacctgcagcccaagcttaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaatcga

SEQ ID NO: 7 - DNA, 121 bp, unidentified
  source 1..121: mol_type = other DNA
aacttgtttattgcagcttataatggttacaaataaagcaatagcatcacaaatttcacaaataaagcatttttttcactgcattctagttgtggtttgtccaaactcatcaatgtatctt

SEQ ID NO: 8 - DNA, 2163 bp, unidentified
  source 1..2163: mol_type = other DNA
ctctaagccctctgcttggagatgctgtaaatacagaacgcaaaatcaccttcgaagttaaagacgcgaagttcttctttactcggcccctcctcccctcccccccgccaattccctccagttacagctagcatccaggtcccgggaggtgaagaaggagacttcggctccagttacagctagcatccgggtcccgatttagaaggagctgccaattacagcgcggttccagggctgagcaaaaagcctgaggagccaagtgggagagggagtaaaactactgaattgggccacaagcaaatgaataaactgaacgactcttaaccaaacctaatatatttaatccaaacacacaagtctttcatttcttccctcctcccttccttctcttactccccaacaccccctcttcaagcacaattaattatatggttagattctactgcgtgatcagccctgttctaggtggtgggcacgccaaggtgaatgagaccaaacaagagtcttgccctcatggggtttacatttggagacagagtcgatctgttgcccaacctggagtgcagtggcgcgatcacagctcactgcagcctcaaactccctggctcaaggggttctcccacctgagcctcccgactagctgggaccacaggtgcacgccacgacgcctgggtttgtttgtttgtttaatagagacgaaggtctcaccatgttatctgggctcaagcgatcatcccccctcctcctcctaaagtactgggattacagtcccaagctatcttgcccgacctgggaaacagacgttaaggaagataacaatctattttcagagagcgagtttataaaaccaatgcaatgggtaaatatgaagtgtgaataggaggagaagctaaagagtggtcggagaatctaatgcaagctacgggagaaagaaactcaagtgcaaatgctgcctcaggaataaacgtaaaaagagactttcaagtgcaaatgctccctcaggaataaaataatcttgagactctcaagtgtaaatgctgcctcgggagaaccgaacggcgagctggagcccatacgcaacgagattagagaggaaggcagaagccagagcacatgaataaatgagcatccattttgtttcagaaatgatcggaaaccatttgtgggtttgtagaagcaggcatgcgtagggaagctacgggattccgccgaggagcgccagagcctgaggcgccctttggttatcgcaagctggctggctcactccgcaccaggtgcaaaagatgcctggggatgcgggaagggaaaggccacatcttcacgccttcgcgcctggcattgtgagcaaccactgagactcattatataacactcgttttcttcttgcaaccctgcgggccgcgcggtcgcgctttctctgccctccgccgggtggacctggagcgcttgagcggtcggcgcgcctggagcagccaggcgggcagtggactagctgctggaccagggaggtgtgggagagcggtggcggcgggtacatgcacgtgaagccattgcgagaactttatccataagtatttcaatgccggtagggacggcaagagaggagggcgggatgtgccacacatctttgacctcaggtttctaacgcctgttttctttctgccctctgcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactca

SEQ ID NO: 9 - DNA, 14205 bp, unidentified
  source 1..14205: mol_type = other DNA
gatccggaataagtctaaatgaatcactttcagttttcctaaacttctatgcctttgcacatcctcttacctctgcctagaatatctttctccttcttttccatctttaaactctcacatcattcttcaagactgggatcagctctcagcatccggaagcctttgcctactagagacaaatgagaatgagtttggtcaccttttcattttcttgtatcattctgtgctttattttgctcttctaagagcgttacatgcttcatttaatccctaaacaactgtttgaggcaagtacagttattatcctaatcatgcaaatgagaaaacagaggcccagacatgttgagtaactttgataaaagttaaagaaccaataagtggaacagttgaggtttgaaccctggcagtctgactgtagagatactatgtttgacctactcccctctgcccccaccccatgtctgcccttagtttctgagcttgttgaatgaatgaacaggtggtagtctttttttgttataagactgatcagaattaagacaggtttaaatttcacgtgtagaattttcaaaactgcaaaggcagtgcaaatctaaaaaaagaatggcattctcaggaaagaggaaaagtaagtgtgagaataataataacaataaccaacaaactttagtaaatttagtaaatgtagtaaatttttacattaaaagcttttggacatacattatcatattttatggccacatgaaatatattataatcccattttgcacataggaaatctgagactggcataaggagcacagagatccaggactttatattttcattcttctaggattttgcacctcaggtcgatatgtatgagtaaactgggagtataatgggctctttaacagaaaaactaggaaagttttcccactattattaattatttacataatatttttttaattttattattatttatactttaagttttagagtacatgtgcacaatgtgcaggtttgttacatatgtatacatgtgccatgttggtgtgctgcacccatcaactcatcatttagcattaggtatatctcctaatgctatccctcccccctcccccctacataagatttataatggataatggacttcaatttctagagcaaaatggccccacccaaggatgccataatccttccagagctctactgcaagatatgagatatacatatctaaaacttgttcttggtatttccaaagcagtcaacttttacacctgtttataatgcatccaaatgttgtttttatatggttgcatctcccatcttcttcaccaatagctatatatatttttcacaagagctgaaagagttcttgatgtaggaatccatggtagagtttcagagaaatccctgaattcactgaaagttttatctagaaatacatgtgcaagtgaacacatcttttttaaaaaaaatcattacctactttcttttttgagaagaaggtatttatttcaacagactcttgaaggagcctactcttcccactctcccacccccattaagaaccactgtaggccgggcacgatggctcatgcctgtaatcccagcactttgggaggctaaggtgggtggatcacctgaggtcaggagttcgagacaagcctagccaacatagtgaaaccccgtctctactaataatacaaaaattagctgggtatggcagcatgtgcctgtaatcccagctactcgggaggctgaggcaggagaattgctcgaacccgggaggcggaggttgcagtgaaccgagagagatcgtgcggtgccatttcactccagcctgggcaacagagcgaaactccatctcaaaaaaacacacaaaacaaacaaacaaaaagaaagaaccattgtattagtgatggaaatgtgttccctccctcccatcctggcaaccactttcttcctcctccatcataaaatatcttaaactaaactaaaataattttatttatcgatagtttgaattttccctatcattgctacacagctaattgagaggtaccccgaggaaaatataaatggtacagtaatgcattgtagattttaataacatacttgacatcccaaattgttttcattggcttcattttaaaaactacatgttttaaaatcaagcagacactaaaagtacaagatatactgggtctacaaggtttaagtcaaccagggattgaaatataacttttaaacagagctggattatccagtaggcagattaagcatgtgcttaaggcatcagcaaagtctgagcaatccattttttaaaacgtagtacatgtttttgataagcttaaaaagtagtagtcacaggaaaaattagaacttttacctccttgcgcttgttatactctttagtgctgtttaacttttctttgtaagtgagggtggtggagggtgcccataatcttttcagggagtaagttcttcttggtctttctttctttctttctttctttttttcttgagaccaagtttcgctcttgtctcccaggctggagtgcaatggcgcgatctcggctcactgcaacctccgccttctcctgggttcaagcgattctcctacatcagcctccgagtagctgggattacaggcatgcgccaccaagccccgctaattttgtattttttagtagagacagggtttcgccatgttggtcaggcttgtctcgaactcctggcctcaggtgatccgcctgtctcggcctcccagaatgctgggattatagacgtgagccaccgcatccggactttccttttatgtaatagtgataattctatccaaagcatttttttttttttttttgagtcggagtctcattctgtcacccaggctggagggtggtggcgcgatctcggcttactgcaacctctgcctcccgggttcaagcgattctcctgcctcagcctcctgagtagctggaattacacacgtgcgccaccatggccagctaatttttgtatttttagtagagacggggtgtcaccattttggccaagctggcctcgaactcctgacctcaggtgatctgcccgcctcggcttcccaaagtgctgggattacaggtgtgagccaccgcgtcctgctccaaagcattttctttctatgcctcaaaacaagattgcaagccagtcctcaaagcggataattcaagagctaacaggtattagcttaggatgtgtggcactgttcttaaggcttatatgtattaatacatcatttaaactcacaacaacccctataaagcagggggcactcatattcccttccccctttataattacgaaaaatgcaaggtattttcagtaggaaagagaaatgtgagaagtgtgaaggagacaggacagtatttgaagctggtctttggatcactgtgcaactctgcttctagaacactgagcactttttctggtctaggaattatgactttgagaatggagtccgtccttccaatgactccctccccattttcctatctgcctacaggcagaattctcccccgtccgtattaaataaacctcatcttttcagagtctgctcttataccaggcaatgtacacgtctgagaaacccttgccccagacagccgttttacacgcaggaggggaaggggaggggaaggagagagcagtccgactctccaaaaggaatcctttgaactagggtttctgacttagtgaaccccgcgctcctgaaaatcaagggttgagggggtagggggacactttctagtcgtacaggtgatttcgattctcggtggggctctcacaactaggaaagaatagttttgctttttcttatgattaaaagaagaagccatactttccctatgacaccaaacaccccgattcaatttggcagttaggaaggttgtatcgcggaggaaggaaacggggcgggggcggatttctttttaacagagtgaacgcactcaaacacgcctttgctggcaggcgggggagcgcggctgggagcagggaggccggagggcggtgtggggggcaggtggggaggagcccagtcctccttccttgccaacgctggctctggcgagggctgcttccggctggtgcccccgggggagacccaacctggggcgacttcaggggtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtgggtagagggtctgcagcgggagcaggggatggcgggcgactctggaggacgaagtttgcaggggaattggaatcaggtagcgcttcgattctccggaaaaaggggaggcttcctggggagttttcagaaggggtttgtaatcacagacctcctcctggcgacgccctgggggcttgggaagccaaggaagaggaatgaggagccacgcgcgtacagatctctcgaatgctgagaagatctgaaggggggaacatatttgtattagatggaagtatgccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattcataacttcgtatagcatacattatacgaagttatacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatgaccaagcttataacttcgtatagcatacattatacgaagttatcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatagacctgcagcccaagcttaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaatcgaaacttgtttattgcagcttataatggttacaaataaagcaatagcatcacaaatttcacaaataaagcatttttttcactgcattctagttgtggtttgtccaaactcatcaatgtatcttactctaagccctctgcttggagatgctgtaaatacagaacgcaaaatcaccttcgaagttaaagacgcgaagttcttctttactcggcccctcctcccctcccccccgccaattccctccagttacagctagcatccaggtcccgggaggtgaagaaggagacttcggctccagttacagctagcatccgggtcccgatttagaaggagctgccaattacagcgcggttccagggctgagcaaaaagcctgaggagccaagtgggagagggagtaaaactactgaattgggccacaagcaaatgaataaactgaacgactcttaaccaaacctaatatatttaatccaaacacacaagtctttcatttcttccctcctcccttccttctcttactccccaacaccccctcttcaagcacaattaattatatggttagattctactgcgtgatcagccctgttctaggtggtgggcacgccaaggtgaatgagaccaaacaagagtcttgccctcatggggtttacatttggagacagagtcgatctgttgcccaacctggagtgcagtggcgcgatcacagctcactgcagcctcaaactccctggctcaaggggttctcccacctgagcctcccgactagctgggaccacaggtgcacgccacgacgcctgggtttgtttgtttgtttaatagagacgaaggtctcaccatgttatctgggctcaagcgatcatcccccctcctcctcctaaagtactgggattacagtcccaagctatcttgcccgacctgggaaacagacgttaaggaagataacaatctattttcagagagcgagtttataaaaccaatgcaatgggtaaatatgaagtgtgaataggaggagaagctaaagagtggtcggagaatctaatgcaagctacgggagaaagaaactcaagtgcaaatgctgcctcaggaataaacgtaaaaagagactttcaagtgcaaatgctccctcaggaataaaataatcttgagactctcaagtgtaaatgctgcctcgggagaaccgaacggcgagctggagcccatacgcaacgagattagagaggaaggcagaagccagagcacatgaataaatgagcatccattttgtttcagaaatgatcggaaaccatttgtgggtttgtagaagcaggcatgcgtagggaagctacgggattccgccgaggagcgccagagcctgaggcgccctttggttatcgcaagctggctggctcactccgcaccaggtgcaaaagatgcctggggatgcgggaagggaaaggccacatcttcacgccttcgcgcctggcattgtgagcaaccactgagactcattatataacactcgttttcttcttgcaaccctgcgggccgcgcggtcgcgctttctctgccctccgccgggtggacctggagcgcttgagcggtcggcgcgcctggagcagccaggcgggcagtggactagctgctggaccagggaggtgtgggagagcggtggcggcgggtacatgcacgtgaagccattgcgagaactttatccataagtatttcaatgccggtagggacggcaagagaggagggcgggatgtgccacacatctttgacctcaggtttctaacgcctgttttctttctgccctctgcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactcagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctggggatcccgacatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactga

SEQ ID NO: 10 - RNA, 200 nt, unidentified
  source 1..200: mol_type = other RNA
mcmamgrartrgrartrgrcrcrarcrgrcrarcrarartrgrtrtrtrtrargrargrcrtrargrararartrargrcrarargrtrtrarararartrarargrgrcrtrargrtrcrcrgrtrtrartrcrararcrtrtrgrararararargrtrgrgrcrarcrcrgrargrtrcrgrgrtrgrcmtmtmtrt

SEQ ID NO: 11 - DNA, 20 bp, unidentified
  source 1..20: mol_type = other DNA
ccatgaccgagatcggcgag

SEQ ID NO: 12 - DNA, 23 bp, unidentified
  source 1..23: mol_type = other DNA
gtgtcagaaacgatgctgtcttc

SEQ ID NO: 13 - DNA, 20 bp, unidentified
  source 1..20: mol_type = other DNA
cgaggagcaggactgaatcg

SEQ ID NO: 14 - DNA, 20 bp, unidentified
  source 1..20: mol_type = other DNA
tcggttggttgggaggtttc

SEQ ID NO: 15 - DNA, 20 bp, unidentified
  source 1..20: mol_type = other DNA
gtctgctgaaactgccaaca

SEQ ID NO: 16 - DNA, 20 bp, unidentified
  source 1..20: mol_type = other DNA
gaggggccgagtaaagaaga

SEQ ID NO: 17 - DNA, 20 bp, unidentified
  source 1..20: mol_type = other DNA
gacttcgtggaggacgactt

SEQ ID NO: 18 - DNA, 20 bp, unidentified
  source 1..20: mol_type = other DNA
agccacgtctctaccttgac

SEQ ID NO: 19 - DNA, 20 bp, unidentified
  source 1..20: mol_type = other DNA
ctcattcagggaggagctct

SEQ ID NO: 20 - DNA, 19 bp, unidentified
  source 1..19: mol_type = other DNA
gccgtacatgcgacagttc

SEQ ID NO: 21 - DNA, 18 bp, unidentified
  source 1..18: mol_type = other DNA
cttcctggacacgctggt

SEQ ID NO: 22 - DNA, 18 bp, unidentified
  source 1..18: mol_type = other DNA
caggtaccgtgcgacatc

SEQ ID NO: 23 - DNA, 18 bp, unidentified
  source 1..18: mol_type = other DNA
ctgaggagctgggccatc

SEQ ID NO: 24 - DNA, 20 bp, unidentified
  source 1..20: mol_type = other DNA
ggggagttttcagaaggggt

SEQ ID NO: 25 - DNA, 20 bp, unidentified
  source 1..20: mol_type = other DNA
tggctcctcattcctcttcc

SEQ ID NO: 26 - DNA, 20 bp, unidentified
  source 1..20: mol_type = other DNA
aatcacagacctcctcctgg

SEQ ID NO: 27 - DNA, 20 bp, unidentified
  source 1..20: mol_type = other DNA
cttggaataaggccggtgtg

SEQ ID NO: 28 - DNA, 20 bp, unidentified
  source 1..20: mol_type = other DNA
aagaagacagggccaggttt

SEQ ID NO: 29 - DNA, 20 bp, unidentified
  source 1..20: mol_type = other DNA
tcttttggcaatgtgagggc